(19) United States
(12) Patent Application Publication
(10) Pub. No.: US 2003/0045002 A1
(43) Pub. Date: Mar. 6, 2003

(54) Cartridge for biochemical analysis unit and method for recording biochemical analysis data in biochemical analysis unit

(75) Inventor: Katsuaki Muraishi, Kanagawa (JP)

Correspondence Address: SUGHRUE MION, PLLC, 2100 PENNSYLVANIA AVENUE, N.W., WASHINGTON, DC, 20037, US

(73) Assignee: FUJI PHOTO FILM CO., LTD.

(21) Appl. No.: 10/224,376

(22) Filed: Aug. 21, 2002

(30) Foreign Application Priority Data
Aug. 28, 2001 (JP) 2001-257464

(51) Int. Cl. C12M 1/34; G01N 33/558; G01N 33/543

(52) U.S. Cl. 436/514; 435/287.2

(57) ABSTRACT

A cartridge for a biochemical analysis unit is adapted for accommodating a biochemical analysis unit and formed with at least one fluid passage for leading a solution to only a plurality of absorptive regions formed in the biochemical analysis unit to be spaced apart from each other. According to thus constituted cartridge, it is possible to forcibly and uniformly feed a reaction solution containing a ligand or a receptor labeled with a labeling substance to the plurality of absorptive regions of the biochemical analysis unit, thereby associating the ligand or the receptor contained in the reaction solution with a receptor or a ligand fixed in the absorptive regions of the biochemical analysis unit. Therefore, it is possible to extremely efficiently associate the ligand or the receptor contained in the reaction solution with the receptor or the ligand fixed in the absorptive regions of the biochemical analysis unit.

### BACKGROUND OF THE INVENTION

The present invention relates to a cartridge for a biochemical analysis unit and a method for recording biochemical analysis data in a biochemical analysis unit and, particularly, to a cartridge for a biochemical analysis unit and a method for recording biochemical analysis data in a biochemical analysis unit which can efficiently associate a ligand or a receptor labeled with a labeling substance with receptors or ligands fixed in a plurality of spot-like regions formed in the biochemical analysis unit to be spaced apart from each other, thereby recording biochemical analysis data in the biochemical analysis unit.

### DESCRIPTION OF THE PRIOR ART

An autoradiographic analyzing system using as a detecting material for detecting radiation a stimulable phosphor which can absorb, store and record the energy of radiation when it is irradiated with radiation and which, when it is then stimulated by an electromagnetic wave having a specified wavelength, can release stimulated emission whose light amount corresponds to the amount of radiation with which it was irradiated is known, which comprises the steps of introducing a radioactively labeled substance into an organism, using the organism or a part of the tissue of the organism as a specimen, superposing the specimen and a stimulable phosphor sheet formed with a stimulable phosphor layer for a certain period of time, storing and recording radiation energy in a stimulable phosphor contained in the stimulable phosphor layer, scanning the stimulable phosphor layer with an electromagnetic wave to excite the stimulable phosphor, photoelectrically detecting the stimulated emission released from the stimulable phosphor to produce digital image signals, effecting image processing on the obtained digital image signals, and reproducing an image on displaying means such as a CRT or the like or a photographic film (see, for example, Japanese Patent Publication No. 1-60784, Japanese Patent Publication No. 1-60782, Japanese Patent Publication No. 4-3952 and the like).

There is further known chemiluminescence analysis system comprising the steps of employing, as a detecting material for light, a stimulable phosphor which can absorb and store the energy of light upon being irradiated therewith and release a stimulated emission whose amount is proportional to that of the received light upon being stimulated with an electromagnetic wave having a specific wavelength range, selectively labeling a fixed high molecular substance such as a protein or a nucleic acid sequence with a labeling substance which generates chemiluminescence emission when it contacts a chemiluminescent substance, contacting the high molecular substance selectively labeled with the labeling substance and the chemiluminescent substance, storing and recording the chemiluminescence emission in the wavelength of visible light generated by the contact of the chemiluminescent substance and the labeling substance in the stimulable phosphor contained in a stimulable phosphor layer formed on a stimulable phosphor sheet, scanning the stimulable phosphor layer with an electromagnetic wave to excite the stimulable phosphor, photoelectrically detecting the stimulated emission released from the stimulable phosphor to produce digital signals, effecting data processing on the obtained digital signals, and reproducing data on displaying means such as a CRT or a recording material such as a photographic film (see for example, U.S. Pat. No. 5,028,793, UK Patent Application 2,246,197 A and the like).

Unlike the system using a photographic film, according to these systems using the stimulable phosphor as a detecting material, development, which is chemical processing, becomes unnecessary. Further, it is possible reproduce a desired image by effecting image processing on the obtained image data and effect quantitative analysis using a computer. Use of a stimulable phosphor in these processes is therefore advantageous.

On the other hand, a fluorescence analyzing system using a fluorescent substance as a labeling substance instead of a radioactive labeling substance in the autoradiographic analyzing system is known. According to this system, it is possible to study a genetic sequence, study the expression level of a gene, and to effect separation or identification of protein or estimation of the molecular weight or properties of protein or the like. For example, this system can perform a process including the steps of distributing a plurality of DNA fragments on a gel support by means of electrophoresis after a fluorescent dye was added to a solution containing a plurality of DNA fragments to be distributed, or distributing a plurality of DNA fragments on a gel support containing a fluorescent dye, or dipping a gel support on which a plurality of DNA fragments have been distributed by means of electrophoresis in a solution containing a fluorescent dye, thereby labeling the electrophoresed DNA fragments, exciting the fluorescent dye by a stimulating ray to cause it to release fluorescence emission, detecting the released fluorescence emission to produce an image and detecting the distribution of the DNA fragments on the gel support. This system can also perform a process including the steps of distributing a pluralty of DNA fragments on a gel support by means of electrophoresis, denaturing the DNA fragments, transferring at least a part of the denatured DNA fragments onto a transfer support such as a nitrocellulose support by the Southern-blotting method, hybridizing a probe prepared by labeling target DNA and DNA or RNA complementary thereto with the denatured DNA fragments, thereby selectively labeling only the DNA fragments complementary to the probe DNA or probe RNA, exciting the fluorescent dye by a stimulating ray to cause it to release fluorescence emission, detecting the released fluorescence emission to produce an image and detecting the distribution of the target DNA on the transfer support. This system can further perform a process including the steps of preparing a DNA probe complementary to DNA containing a target gene labeled by a labeling substance, hybridizing it with DNA on a transfer support, combining an enzyme with the complementary DNA labeled by a labeling substance, causing the enzyme to contact a fluorescent substance, transforming the fluorescent substance to a fluorescent substance having fluorescence emission releasing property, exciting the thus produced fluorescent substance by a stimulating ray to release fluorescence emission, detecting the fluorescence emission to produce an image and detecting the distribution of the target DNA on the transfer support. This fluorescence detecting system is advantageous in that a genetic sequence or the like can be easily detected without using a radioactive substance.

Similarly, there is known a chemiluminescence detecting system comprising the steps of fixing a substance derived from a living organism such as a protein or a nucleic acid sequence on a support, selectively labeling the substance derived from a living organism with a labeling substance which generates chemiluminescence emission when it contacts a chemiluminescent substrate, contacting the substance derived from a living organism and selectively labeled with the labeling substance and the chemiluminescent substrate, photoelectrically detecting the chemiluminescence emission in the wavelength of visible light generated by the contact of the chemiluminescent substrate and the labeling substance to produce digital image signals, effecting image processing thereon, and reproducing a chemiluminescent image on a display means such as a CRT or a recording material such as a photographic film, thereby obtaining information relating to the high molecular substance such as genetic information

Further, a micro-array analyzing system has been recently developed, which comprises the steps of using a spotting device to drop at different positions on the surface of a carrier such as a slide glass plate, a membrane filter or the like specific binding substances, which can specifically bind with a substance derived from a living organism such as a cell, virus, hormone, tumor marker, enzyme, antibody, antigen, abzyme, other protein, a nuclear acid, cDNA, DNA, RNA or the like and whose sequence, base length, composition and the like are known, thereby forming a number of independent spots, specifically binding the specific binding substances using a hybridization method or the like with a substance derived from a living organism such as a cell, virus, hormone, tumor marker, enzyme, antibody, antigen, abzyme, other protein, a nuclear acid, cDNA, DNA or mRNA by extraction, isolation or the like and optionally further subjected to chemical processing, chemical modification or the like and which is labeled with a labeling substance such as a fluorescent substance, dye or the like, thereby forming a micro-array, irradiating the micro-array with a stimulating ray, photoelectrically detecting light such as fluorescence emission released from a labeling substance such as a fluorescent substance, dye or the like, and analyzing the substance derived from a living organism. This micro-array analyzing system is advantageous in that a substance derived from a living organism can be analyzed in a short time period by forming a number of spots of specific binding substances at different positions of the surface of a carrier such as a slide glass plate at a high density and hybridizing them with a substance derived from a living organism and labeled with a labeling substance.

In addition, a macro-array analyzing system using a radioactive labeling substance as a labeling substance has been further developed, which comprises the steps of using a spotting device to drop at different positions on the surface of a carrier such as a membrane filter or the like specific binding substances, which can specifically bind with a substance derived from a living organism such as a cell, virus, hormone, tumor marker, enzyme, antibody, antigen, abzyme, other protein, a nuclear acid, cDNA, DNA, RNA or the like and whose sequence, base length, composition and the like are known, thereby forming a number of independent spots, specifically binding the specific binding substance using a hybridization method or the like with a substance derived from a living organism such as a cell, virus, hormone, tumor marker, enzyme, antibody, antigen, abzyme, other protein, a nuclear acid, cDNA, DNA or mRNA by extraction, isolation or the like and optionally further subjected to chemical processing, chemical modification or the like and which is labeled with a radioactive labeling substance, thereby forming a macro-array, superposing the macro-array and a stimulable phosphor sheet formed with a stimulable phosphor layer, exposing the stimulable phosphor layer to a radioactive labeling substance, irradiating the stimulable phosphor layer with a stimulating ray to excite the stimulable phosphor, photoelectrically detecting the stimulated emission released from the stimulable phosphor to produce biochemical analysis data, and analyzing the substance derived from a living organism.

However, in the micro-array analyzing system and the macro-array analyzing system, it is required to produce biochemical analysis data by dropping a solution containing specific binding substances at different positions on the surface of a biochemical analysis unit such as a membrane filter or the like to form a number of spot-like regions, hybridizing a substance derived from a living organism and labeled with a labeling substance such as a radioactive labeling substance, a fluorescent substance and a labeling substance which generates chemiluminescence emission when it contacts a chemiluminescent substrate with the specific binding substances contained in the spot-like regions, thereby selectively labeling the spot-like regions, exposing a stimulable phosphor layer of a stimulable phosphor sheet to a radioactive labeling substance selectively contained in the spot-like regions, scanning the thus exposed stimulable phosphor layer with a stimulating ray, thereby exciting stimulable phosphor contained in the stimulable phosphor layer and photoelectrically detecting stimulated emission released from the stimulable phosphor, or scanning a number of the spot-like regions with a stimulating ray, thereby exciting a fluorescent substance contained in a number of the spot-like regions and photoelectrically detecting fluorescence emission released from the fluorescent substance, or bringing a labeling substance contained in a number of the spot-like regions into contact with a chemiluminescent substrate and photoelectrically detecting chemiluminescence emission released from the labeling substance.

Conventionally, hybridization of specific binding substances and a substance derived from a living organism was performed by an experimenter manually inserting a biochemical analysis unit formed with a number of the spot-like regions containing specific binding substances such as a membrane filter into a hybridization bag, pouring a hybridization solution containing a substance derived from a living organism and labeled with a labeling substance such as a radioactive labeling substance, a fluorescent substance or a labeling substance which generates chemiluminescence emission when it contacts a chemiluminescent substrate into the hybridization bag, vibrating the hybridization bag, thereby moving the substance derived from a living organism by convection or diffusion, hybridizing the substance derived from a living organism with the specific binding substances, removing the biochemical analysis unit from the hybridization bag, and inserting the biochemical analysis unit in a container filled with a cleaning solution, thereby cleaning the biochemical analysis unit.

However, in the case where specific binding substances and a substance derived from a living organism are hybridized by an experimenter manually inserting a biochemical analysis unit into a hybridization bag, pouring a hybridization solution into the hybridization bag, and vibrating the hybridization bag, it is difficult to bring the hybridization solution into uniform contact with a number of the spot-like regions containing specific binding substances and, therefore, specific binding substances and a substance derived from a living organism cannot be effectively hybridized.

Further, in the case of an experimenter manually inserting a biochemical analysis unit into a hybridization bag by, pouring a hybridization solution into the hybridization bag, vibrating the hybridization bag, hybridizing specific binding substances and a substance derived from a living organism, removing the biochemical analysis unit from the hybridization bag, and inserting the biochemical analysis unit in a container filled with a cleaning solution, thereby cleaning the biochemical analysis unit, the results of the hybridization differ among different experimenters and the repeatability of the hybridization is inevitably lowered. Moreover, even when the same experimenter performs hybridization, different results may be obtained.

Furthermore, a substance derived from a living organism should not be bonded with specific binding substances by hybridization may be bonded with the specific binding substances. In such cases, when biochemical analysis data are produced by bringing a biochemical analysis unit such as a membrane filter into close contact with a stimulable phosphor sheet formed with a stimulable phosphor layer containing stimulable phosphor, thereby exposing the stimulable phosphor layer, irradiating the stimulable phosphor layer with a stimulating ray and photoelectrically detecting stimulated emission released from the stimulable phosphor layer, or irradiating the biochemical analysis unit such as a membrane filter with a stimulating ray and photoelectrically detecting fluorescence emission released from a fluorescent substance, or photoelectrically detecting chemiluminescence emission released from a biochemical analysis unit such as a membrane filter, noise is generated in the biochemical analysis data and quantitative accuracy of quantitative analysis is lowered.

In the case where a receptor and a ligand are associated as in the case of fixing antigens or antibodies in a biochemical analysis unit such as a membrane filter and binding an antibody or an antigen to the thus fixed antigens or antibodies by an antigen-antibody reaction, the same problems occur, and in the case of hybridizing a probe DNA labeled with a hapten such as digoxigenin with a target DNA fixed in a biochemical analysis unit such as a membrane filter, binding an antibody for the hapten such as digoxigenin labeled with an enzyme which generates chemiluminescent emission when it contacts a chemiluminescent substrate or an antibody for the hapten such as digoxigenin labeled with an enzyme which generates fluorescence emission when it contacts a fluorescent substrate with the hapten labeling the probe DNA by an an antigen-antibody reaction, thereby labeling the target DNA, the same problems also occur.

### SUMMARY OF THE INVENTION

It is therefore an object of the present invention to provide a cartridge for a biochemical analysis unit and a method for recording biochemical analysis data in a biochemical analysis unit which can efficiently associate a ligand or a receptor labeled with a labeling substance with receptors or ligands fixed in a plurality of spot-like regions formed in the biochemical analysis unit to be spaced apart from each other, thereby recording biochemical analysis data in the biochemical analysis unit.

The above other objects of the present invention can be accomplished by a cartridge for a biochemical analysis unit being adapted for accommodating a biochemical analysis unit and formed with at least one fluid passage for leading a solution to only a plurality of absorptive regions formed in the biochemical analysis unit to be spaced apart from each other.

According to the present invention, since the cartridge for accommodating a biochemical analysis unit is formed with at least one fluid passage for leading a solution to only a plurality of absorptive regions formed in the biochemical analysis unit to be spaced apart from each other, it is possible to forcibly and uniformly feed a reaction solution containing a ligand or a receptor labeled with a labeling substance to the plurality of absorptive regions of the biochemical analysis unit, thereby associating the ligand or the receptor contained in the reaction solution with a receptor or a ligand fixed in the absorptive regions of the biochemical analysis unit and, therefore, in comparison with the case of moving a ligand or a receptor by convection or diffusion and associating it with a receptor or a ligand fixed in the absorptive regions of the biochemical analysis unit, it is possible to extremely efficiently associate the ligand or the receptor contained in the reaction solution with the receptor or the ligand fixed in the absorptive regions of the biochemical analysis unit.

Furthermore, according to the present invention, since the cartridge for accommodating a biochemical analysis unit is formed with at least one fluid passage for leading a solution to only a plurality of absorptive regions formed in the biochemical analysis unit to be spaced apart from each other, it is possible to forcibly and uniformly feed a cleaning solution to the plurality of absorptive regions of the biochemical analysis unit, thereby cleaning the plurality of absorptive regions of the biochemical analysis unit. Therefore, in comparison with the case of moving the cleaning solution by convection or diffusion and cleaning the plurality of absorptive regions of the biochemical analysis unit therewith, since it is possible to extremely efficiently clean the plurality of absorptive regions of the biochemical analysis unit, even in the case where a ligand or a receptor which should not be associated with the receptors or the ligands fixed in the plurality of absorptive regions of the biochemical analysis unit has been bonded therewith in the course the receptor-ligand association reaction, it is possible to effectively peel off and remove the ligand or the receptor which should not be associated with the receptors or the ligands fixed in the plurality of absorptive regions of the biochemical analysis unit from the plurality of absorptive regions. Therefore, since the ligand or the receptor which is to be associated with the receptors or the ligands fixed in the individual absorptive regions of the biochemical analysis unit can be associated therewith in a desired manner, it is possible to effectively prevent noise from being generated in biochemical analysis data and to produce biochemical analysis data having an excellent high quantitative characteristic with excellent repeatability.

Moreover, according to the present invention, since a reaction solution containing a ligand or a receptor can be forcibly fed to only the plurality of absorptive regions of the biochemical analysis unit, the ligand or the receptor can be reliably prevented from adhering to portions other than the plurality of absorptive regions of the biochemical analysis unit. Therefore, since it is sufficient for a cleaning solution to be fed to only the plurality of absorptive regions of the biochemical analysis unit to clean them, the efficiency of the cleaning operation can be markedly improved.

Further, according to the present invention, since the receptor-ligand association reaction and the cleaning can be effected within micro-regions by determining the size of the at least one fluid passage for leading a solution to the plurality of absorptive regions formed in the biochemical analysis unit to be spaced apart from each other sufficiently small, the reaction can be facilitated in accordance with the principle of a micro-reactor and, therefore, the efficiency of the receptor-ligand association reaction and the efficiency of the cleaning operation can be markedly improved.

In a preferred aspect of the present invention, the at least one fluid passage is formed so as to cut through the plurality of absorptive regions formed in the biochemical analysis unit to be spaced apart from each other.

According to this preferred aspect of the present invention, since the at least one fluid passage is formed so as to cut through the plurality of absorptive regions formed in the biochemical analysis unit to be spaced apart from each other, a reaction solution containing a ligand or a receptor can be fed to the plurality of absorptive regions formed in the biochemical analysis unit to be spaced from each other so as to pass through the plurality of absorptive regions, thereby selectively associating the ligand or the receptor contained in the reaction solution with the receptor or the ligand fixed in the plurality of absorptive regions of the biochemical analysis unit. Therefore, since it is possible to markedly increase the moving rate of the ligand or the receptor in comparison with the case of moving a ligand or a receptor by convection or diffusion and associating it with the receptor or the ligand fixed in the absorptive regions of the biochemical analysis unit, it is possible to markedly increase the reaction rate of association of the receptors or the ligands fixed in the plurality of absorptive regions of the biochemical analysis unit and the ligand or the receptor contained in the reaction solution and to markedly increase the possibility of association of the ligand or the receptor contained in the reaction solution with the receptors or the ligands fixed in deep portions of the plurality of absorptive regions of the biochemical analysis unit. Therefore, the ligand or the receptor contained in the reaction solution can be associated with the receptors or the ligands fixed in the plurality of absorptive regions of the biochemical analysis unit in a desired manner.

Furthermore, according to this preferred aspect of the present invention, since the at least one fluid passage is formed so as to cut through the plurality of absorptive regions formed in the biochemical analysis unit to be spaced apart from each other, a cleaning solution can be fed to the plurality of absorptive regions of the biochemical analysis unit so as to pass through the plurality of absorptive regions, thereby cleaning the plurality of absorptive regions of the biochemical analysis unit therewith. Therefore, since it is possible to efficiently clean the plurality of absorptive regions of the biochemical analysis unit with the cleaning solution in comparison with the case of moving the cleaning solution by convection or diffusion and cleaning the plurality of absorptive regions of the biochemical analysis unit therewith, even in the case where a ligand or a receptor which should not be associated with the receptors or the ligands fixed in the plurality of absorptive regions of the biochemical analysis unit has been bonded therewith in the course of the receptor-ligand association reaction, it is possible to effectively peel off and remove the ligand or the receptor which should not be associated with the receptors or the ligands fixed in the plurality of absorptive regions of the biochemical analysis unit from the plurality of absorptive regions. Therefore, since the ligand or the receptor which is to be associated with the receptors or the ligands fixed in the individual absorptive regions of the biochemical analysis unit can be associated therewith in a desired manner, it is possible to effectively prevent noise from being generated in biochemical analysis data and to produce biochemical analysis data having an excellent high quantitative characteristic with excellent repeatability.

In a preferred aspect of the present invention, a plurality of fluid passages are formed.

According to this preferred aspect of the present invention, since a plurality of fluid passages are formed, it is possible to forcibly and uniformly feed a reaction solution containing a ligand or a receptor labeled with a labeling substance to the plurality of absorptive regions of the biochemical analysis unit through the plurality of fluid passages, thereby associating the ligand or the receptor contained in the reaction solution with a receptor or a ligand fixed in the absorptive regions of the biochemical analysis unit and, therefore, in comparison with the case of moving a ligand or a receptor by convection or diffusion and associating it with a receptor or a ligand fixed in the absorptive regions of the biochemical analysis unit, it is possible to extremely efficiently associate the ligand or the receptor contained in the reaction solution with the receptor or the ligand fixed in the absorptive regions of the biochemical analysis unit.

Further, according this preferred aspect of the present invention, since a plurality of fluid passages are formed, it is possible to forcibly and uniformly feed a cleaning solution to the plurality of absorptive regions of the biochemical analysis unit through the plurality of fluid passages, thereby cleaning the plurality of absorptive regions of the biochemical analysis unit. Therefore, in comparison with the case of moving the cleaning solution by convection or diffusion and cleaning the plurality of absorptive regions of the biochemical analysis unit therewith, since it is possible to extremely efficiently clean the plurality of absorptive regions of the biochemical analysis unit, even in the case where a ligand or a receptor which should not be associated with the receptors or the ligands fixed in the plurality of absorptive regions of the biochemical analysis unit has been bonded therewith in the course of the receptor-ligand association reaction, it is possible to effectively peel off and remove the ligand or the receptor which should not be associated with the receptors or the ligands fixed in the plurality of absorptive regions of the biochemical analysis unit from the plurality of absorptive regions. Therefore, since the ligand or the receptor which is to be associated with the receptors or the ligands fixed in the individual absorptive regions of the biochemical analysis unit can be associated therewith in a desired manner, it is possible to effectively prevent noise from being generated in biochemical analysis data and to produce biochemical analysis data having an excellent high quantitative characteristic with excellent repeatability.

Furthermore, according this preferred aspect of the present invention, since a reaction solution containing a ligand or a receptor can be forcibly fed to only the plurality of absorptive regions of the biochemical analysis unit through the plurality of fluid passages, the ligand or the receptor can be reliably prevented from adhering to portions other than the plurality of absorptive regions of the biochemical analysis unit. Therefore, since it is sufficient for a cleaning solution to be fed to only the plurality of absorptive regions of the biochemical analysis unit to clean them, the efficiency of the cleaning operation can be markedly improved.

Moreover, according this preferred aspect of the present invention, since the receptor-ligand association reaction and the cleaning can be effected within micro-regions by determining the size of each of the plurality of fluid passages for leading a solution to the plurality of absorptive regions formed in the biochemical analysis unit to be spaced apart from each other sufficiently small, the reaction can be facilitated in accordance with the principle of a micro-reactor and, therefore, the efficiency of the receptor-ligand association reaction and the efficiency of the cleaning operation can be markedly improved.

In a preferred aspect of the present invention, the plurality of absorptive regions are two-dimensionally formed in the biochemical analysis unit to be spaced apart from each other and a fluid passage is formed for each line of the plurality of absorptive regions so that the fluid passage can successively lead a solution to the plurality of absorptive regions constituting each line.

According to this preferred aspect of the present invention, since the plurality of absorptive regions are two-dimensionally formed in the biochemical analysis unit to be spaced apart from each other and a fluid passage is formed for each line of the plurality of absorptive regions so that the fluid passage can successively lead a solution to the plurality of absorptive regions constituting each line, it is possible to forcibly and uniformly feed a reaction solution containing a ligand or a receptor to the plurality of absorptive regions constituting each line of the plurality of absorptive regions of the biochemical analysis unit through the fluid passage formed for the line, thereby associating the ligand or the receptor contained in the reaction solution with a receptor or a ligand fixed in the absorptive regions of the biochemical analysis unit and, therefore, in comparison with the case of moving a ligand or a receptor by convection or diffusion and associating it with a receptor or a ligand fixed in the absorptive regions of the biochemical analysis unit, it is possible to extremely efficiently associate the ligand or the receptor contained in the reaction solution with the receptor or the ligand fixed in the absorptive regions of the biochemical analysis unit.

Further, according to this preferred aspect of the present invention, since the plurality of absorptive regions are two-dimensionally formed in the biochemical analysis unit to be spaced apart from each other and a fluid passage is formed for each line of the plurality of absorptive regions so that the fluid passage can successively lead a solution to the plurality of absorptive regions constituting each line, it is possible to forcibly and uniformly feed a cleaning solution to the plurality of absorptive regions constituting each line of the plurality of absorptive regions of the biochemical analysis unit through the fluid passage formed for the line, thereby cleaning the plurality of absorptive regions of the biochemical analysis unit. Therefore, in comparison with the case of moving the cleaning solution by convection or diffusion and cleaning the plurality of absorptive regions of the biochemical analysis unit therewith, since it is possible to extremely efficiently clean the plurality of absorptive regions of the biochemical analysis unit, even in the case where a ligand or a receptor which should not be associated with the receptors or the ligands fixed in the plurality of absorptive regions of the biochemical analysis unit has been bonded therewith in the course of the receptor-ligand association reaction, it is possible to effectively peel off and remove the ligand or the receptor which should not be associated with the receptors or the ligands fixed in the plurality of absorptive regions of the biochemical analysis unit from the plurality of absorptive regions. Therefore, since the ligand or the receptor which is to be associated with the receptors or the ligands fixed in the individual absorptive regions of the biochemical analysis unit can be associated therewith in a desired manner, it is possible to effectively prevent noise from being generated in biochemical analysis data and to produce biochemical analysis data having an excellent high quantitative characteristic with excellent repeatability.

Furthermore, according to this preferred aspect of the present invention, since a reaction solution containing a ligand or a receptor can be forcibly fed to only the plurality of absorptive regions of the biochemical analysis unit, the ligand or the receptor can be reliably prevented from adhering to portions other than the plurality of absorptive regions of the biochemical analysis unit. Therefore, since it is sufficient for a cleaning solution to be fed to only the plurality of absorptive regions of the biochemical analysis unit to clean them, the efficiency of the cleaning operation can be markedly improved.

Moreover, according to this preferred aspect of the present invention, since the receptor-ligand association reaction and the cleaning can be effected within micro-regions by determining the size of the fluid passage for leading a solution to each line of the plurality of absorptive regions formed in the biochemical analysis unit to be spaced apart from each other sufficiently small, the reaction can be facilitated in accordance with the principle of a micro-reactor and, therefore, the efficiency of the receptor-ligand association reaction and the efficiency of the cleaning operation can be markedly improved.

In a preferred aspect of the present invention, the fluid passage formed for each line of the plurality of absorptive regions is formed so as to sequentially cross the absorptive regions constituting the line.

According to this preferred aspect of the present invention, since the fluid passage formed for each line of the plurality of absorptive regions is formed so as to sequentially cross the absorptive regions constituting the line, a reaction solution containing a ligand or a receptor can be fed to the plurality of absorptive regions formed in the biochemical analysis unit to be spaced from each other so as to cut through the plurality of absorptive regions, thereby selectively associating the ligand or the receptor contained in the reaction solution with the receptor or the ligand fixed in the plurality of absorptive regions of the biochemical analysis unit. Therefore, since it is possible to markedly increase the moving rate of the ligand or the receptor in comparison with the case of moving a ligand or a receptor by convection or diffusion and associating it with the receptor or the ligand fixed in the absorptive regions of the biochemical analysis unit, it is possible to markedly increase the reaction rate of association of the receptors or the ligands fixed in the plurality of absorptive regions of the biochemical analysis unit and the ligand or the receptor contained in the reaction solution and to markedly increase the possibility of association of the ligand or the receptor contained in the reaction solution with the receptors or the ligands fixed in deep portions of the plurality of absorptive regions of the biochemical analysis unit. Therefore, the ligand or the receptor contained in the reaction solution can be associated with the receptors or the ligands fixed in the plurality of absorptive regions of the biochemical analysis unit in a desired manner.

Furthermore, according to this preferred aspect of the present invention, since the fluid passage formed for each line of the plurality of absorptive regions is formed so as to sequentially cross the absorptive regions constituting the line, a cleaning solution can be fed to the plurality of absorptive regions of the biochemical analysis unit so as to cut through the plurality of absorptive regions, thereby cleaning the plurality of absorptive regions of the biochemical analysis unit therewith. Therefore, since it is possible to efficiently clean the plurality of absorptive regions of the biochemical analysis unit with the cleaning solution in comparison with the case of moving the cleaning solution by convection or diffusion and cleaning the plurality of absorptive regions of the biochemical analysis unit therewith, even in the case where a ligand or a receptor which should not be associated with the receptors or the ligands fixed in the plurality of absorptive regions of the biochemical analysis unit has been bonded therewith in the course of the receptor-ligand association reaction, it is possible to effectively peel off and remove the ligand or the receptor which should not be associated with the receptors or the ligands fixed in the plurality of absorptive regions of the biochemical analysis unit from the plurality of absorptive regions. Therefore, since the ligand or the receptor which is to be associated with the receptors or the ligands fixed in the individual absorptive regions of the biochemical analysis unit can be associated therewith in a desired manner, it is possible to effectively prevent noise from being generated in biochemical analysis data and to produce biochemical analysis data having an excellent high quantitative characteristic with excellent repeatability.

In another preferred aspect of the present invention, the plurality of fluid passages are disposed on one side of the biochemical analysis unit held in the cartridge.

In a preferred aspect of the present invention, the plurality of absorptive regions are two-dimensionally formed in the biochemical analysis unit to be spaced apart from each other and a fluid passage is formed for each of the absorptive regions so as to feed a solution thereto.

According to this preferred aspect of the present invention, since the plurality of absorptive regions are two-dimensionally formed in the biochemical analysis unit to be spaced apart from each other and a fluid passage is formed for each of the absorptive regions so as to feed a solution thereto, it is possible to forcibly and uniformly feed a reaction solution containing a ligand or a receptor labeled with a labeling substance to the plurality of absorptive regions of the biochemical analysis unit through each of the passages, thereby associating the ligand or the receptor contained in the reaction solution with a receptor or a ligand fixed in the absorptive regions of the biochemical analysis unit and, therefore, in comparison with the case of moving a ligand or a receptor by convection or diffusion and associating it with a receptor or a ligand fixed in the absorptive regions of the biochemical analysis unit, it is possible to extremely efficiently associate the ligand or the receptor contained in the reaction solution with the receptor or the ligand fixed in the absorptive regions of the biochemical analysis unit.

Furthermore, according to this preferred aspect of the present invention, since the plurality of absorptive regions are two-dimensionally formed in the biochemical analysis unit to be spaced apart from each other and a fluid passage is formed for each of the absorptive regions so as to feed a solution thereto, it is possible to forcibly and uniformly feed a cleaning solution to the plurality of absorptive regions of the biochemical analysis unit through each of the passages, thereby cleaning the plurality of absorptive regions of the biochemical analysis unit. Therefore, in comparison with the case of moving the cleaning solution by convection or diffusion and cleaning the plurality of absorptive regions of the biochemical analysis unit therewith, since it is possible to extremely efficiently clean the plurality of absorptive regions of the biochemical analysis unit, even in the case where a ligand or a receptor which should not be associated with the receptors or the ligands fixed in the plurality of absorptive regions of the biochemical analysis unit has been bonded therewith in the course of the receptor-ligand association reaction, it is possible to effectively peel off and remove the ligand or the receptor which should not be associated with the receptors or the ligands fixed in the plurality of absorptive regions of the biochemical analysis unit from the plurality of absorptive regions. Therefore, since the ligand or the receptor which is to be associated with the receptors or the ligands fixed in the individual absorptive regions of the biochemical analysis unit can be associated therewith in a desired manner, it is possible to effectively prevent noise from being generated in biochemical analysis data and to produce biochemical analysis data having an excellent high quantitative characteristic with excellent repeatability.

Furthermore, according to this preferred aspect of the present invention, since the plurality of absorptive regions are two-dimensionally formed in the biochemical analysis unit to be spaced apart from each other and a fluid passage is formed for each of the absorptive regions so as to feed a solution thereto, a reaction solution containing a ligand or a receptor can be forcibly fed to only the plurality of absorptive regions of the biochemical analysis unit. Therefore, since the ligand or the receptor can be reliably prevented from adhering to portions other than the plurality of absorptive regions of the biochemical analysis unit, it is sufficient for a cleaning solution to be fed to only the plurality of absorptive regions of the biochemical analysis unit to clean them and the efficiency of the cleaning operation can be markedly improved.

Moreover, according to this preferred aspect of the present invention, since the plurality of absorptive regions are two-dimensionally formed in the biochemical analysis unit to be spaced apart from each other and a fluid passage is formed for each of the absorptive regions so as to feed a solution thereto, the receptor-ligand association reaction and the cleaning can be effected within micro-regions by determining the size of the at least one fluid passage for leading a solution to the plurality of absorptive regions formed in the biochemical analysis unit to be spaced apart from each other sufficiently small. Therefore, since the reaction can be facilitated in accordance with the principle of a micro-reactor, the efficiency of the receptor-ligand association reaction and the efficiency of the cleaning operation can be markedly improved.

In a further preferred aspect of the present invention, the fluid passage formed for each of the absorptive regions is formed so as to cut through the corresponding absorptive region.

According to this preferred aspect of the present invention, since the fluid passage formed for each of the absorptive regions is formed so as to cut through the corresponding absorptive region, a reaction solution containing a ligand or a receptor can be fed through each of the fluid passages to the plurality of absorptive regions formed in the biochemical analysis unit to be spaced from each other so as to cut through the plurality of absorptive regions, thereby selectively associating the ligand or the receptor contained in the reaction solution with the receptor or the ligand fixed in the plurality of absorptive regions of the biochemical analysis unit. Therefore, since it is possible to markedly increase the moving rate of the ligand or the receptor in comparison with the case of moving a ligand or a receptor by convection or diffusion and associating it with the receptor or the ligand fixed in the absorptive regions of the biochemical analysis unit, it is possible to markedly increase the reaction rate of association of the receptors or the ligands fixed in the plurality of absorptive regions of the biochemical analysis unit and the ligand or the receptor contained in the reaction solution and to markedly increase the possibility of association of the ligand or the receptor contained in the reaction solution with the receptors or the ligands fixed in deep portions of the plurality of absorptive regions of the biochemical analysis unit. Therefore, the ligand or the receptor contained in the reaction solution can be associated with the receptors or the ligands fixed in the plurality of absorptive regions of the biochemical analysis unit in a desired manner.

Furthermore, according to this preferred aspect of the present invention, since the fluid passage formed for each of the absorptive regions is formed so as to cut through the corresponding absorptive region, a cleaning solution can be fed through each of the fluid passages to the plurality of absorptive regions of the biochemical analysis unit so as to cut through the plurality of absorptive regions, thereby cleaning the plurality of absorptive regions of the biochemical analysis unit therewith. Therefore, since it is possible to efficiently clean the plurality of absorptive regions of the biochemical analysis unit with the cleaning solution in comparison with the case of moving the cleaning solution by convection or diffusion and cleaning the plurality of absorptive regions of the biochemical analysis unit therewith, even in the case where a ligand or a receptor which should not be associated with the receptors or the ligands fixed in the plurality of absorptive regions of the biochemical analysis unit has been bonded therewith in the course of the receptor-ligand association reaction, it is possible to effectively peel off and remove the ligand or the receptor which should not be associated with the receptors or the ligands fixed in the plurality of absorptive regions of the biochemical analysis unit from the plurality of absorptive regions. Therefore, since the ligand or the receptor which is to be associated with the receptors or the ligands fixed in the individual absorptive regions of the biochemical analysis unit can be associated therewith in a desired manner, it is possible to effectively prevent noise from being generated in biochemical analysis data and to produce biochemical analysis data having an excellent high quantitative characteristic with excellent repeatability.

In another preferred aspect of the present invention, the fluid passage formed for each of the absorptive regions is disposed on one side of the biochemical analysis unit held in the cartridge.

In the present invention, in the case where the at least one fluid passage or each of the fluid passages is formed so as to cut through the absorptive region(s) of the biochemical analysis unit, it is preferable for a portion of the fluid passage facing the absorptive region of the biochemical analysis unit to have a cross sectional area of 0.2 mm² or less, more preferably, 0.07 mm² or less.

In the case where the at least one fluid passage or each of the fluid passages is formed so as to cut through the absorptive region(s) of the biochemical analysis unit, if the portion of the fluid passage facing the absorptive region of the biochemical analysis unit has a cross sectional area of 0.2 mm² or less, since the reaction occurs within a micro-area, the reaction can be facilitated in accordance with the principle of a micro-reactor and, therefore, the efficiency of the receptor-ligand association reaction and the efficiency of the cleaning operation can be markedly improved. To the contrary, it is not preferable for a cross sectional area of the portion of the fluid passage facing the absorptive region of the biochemical analysis unit to exceed 0.2 mm² because the reaction cannot be sufficiently facilitated based on the principle of a micro-reactor.

In the present invention, in the case where the at least one fluid passage or each of the fluid passages is formed on one side of the biochemical analysis unit held in the cartridge, it is preferable for a portion of the fluid passage facing the absorptive region of the biochemical analysis unit to have a length of 0.5 mm or less, more preferably, 0.1 mm or less.

In the case where the at least one fluid passage or each of the fluid passages is formed on one side of the biochemical analysis unit held in the cartridge, if the length of the portion of the fluid passage facing the absorptive region of the biochemical analysis unit, namely, the passage length, is 0.5 mm or less, since the reaction occurs within a micro-area, the reaction can be facilitated in accordance with the principle of a micro-reactor and, therefore, the efficiency of the receptor-ligand association reaction and the efficiency of the cleaning operation can be markedly improved. To the contrary, it is not preferable for the length of the portion of the fluid passage facing the absorptive region of the biochemical analysis unit, namely, the passage length, to exceeed 0.5 mm because the reaction cannot be sufficiently facilitated based on the principle of a micro-reactor.

The above and other objects of the present invention can be also accomplished by a method for recording biochemical analysis data in a biochemical analysis unit comprising the steps of accommodating a biochemical analysis unit including a substrate formed with a plurality of absorptive regions to be spaced apart from each other in which receptors or ligands are fixed in a cartridge and feeding a reaction solution containing a ligand or a receptor labeled with a labeling substance only to the absorptive regions of the biochemical analysis unit through at least one fluid passage formed in the cartridge, thereby selectively associating the ligand or the receptor contained in the reaction solution with the receptors or the ligands fixed in the plurality of the absorptive regions of the biochemical analysis unit.

In the present invention, the receptor-ligand association reaction includes a hybridization reaction and an antigen-antibody reaction.

According to the present invention, since biochemical analysis data are recorded in a biochemical analysis unit by accommodating a biochemical analysis unit including a substrate formed with a plurality of absorptive regions to be spaced apart from each other in which receptors or ligands are fixed in a cartridge and feeding a reaction solution containing a ligand or a receptor labeled with a labeling substance only to the absorptive regions of the biochemical analysis unit through at least one fluid passage formed in the cartridge, thereby selectively associating the ligand or the receptor contained in the reaction solution with the receptors or the ligands fixed in the plurality of the absorptive regions of the biochemical analysis unit, it is possible to extremely efficiently associate the ligand or the receptor contained in the reaction solution with the receptor or the ligand fixed in the absorptive regions of the biochemical analysis unit in comparison with the case of moving a ligand or a receptor by convection or diffusion and associating it with a receptor or a ligand fixed in the absorptive regions of the biochemical analysis unit.

Further, according to the present invention, since a reaction solution containing a ligand or a receptor can be forcibly fed to only the plurality of absorptive regions of the biochemical analysis unit, the ligand or the receptor can be reliably prevented from adhering to portions other than the plurality of absorptive regions of the biochemical analysis unit. Therefore, since it is sufficient for a cleaning solution to be fed to only the plurality of absorptive regions of the biochemical analysis unit to clean them, the efficiency of the cleaning operation can be markedly improved.

Moreover, since the receptor-ligand association reaction can be effected within micro-regions by determining the size of the at least one fluid passage for leading a solution to the plurality of absorptive regions formed in the biochemical analysis unit to be spaced apart from each other sufficiently small, the reaction can be facilitated in accordance with the principle of a micro-reactor and, therefore, the efficiency of the receptor-ligand association reaction can be markedly improved.

In a preferred aspect of the present invention, the method for recording biochemical analysis data in a biochemical analysis unit further comprises the step of feeding a cleaning solution only to the absorptive regions of the biochemical analysis unit through at least one fluid passage formed in the cartridge, thereby cleaning the plurality of absorptive regions of the biochemical analysis unit in which the receptors or the ligands are fixed with the cleaning solution.

According to this preferred aspect of the present invention, since a cleaning solution is fed only to the absorptive regions of the biochemical analysis unit through at least one fluid passage formed in the cartridge after the receptor-ligand association reaction was completed, thereby cleaning the plurality of absorptive regions of the biochemical analysis unit in which the receptors or the ligands are fixed with the cleaning solution, it is possible to extremely efficiently clean the plurality of absorptive regions of the biochemical analysis unit in comparison with the case of moving the cleaning solution by convection or diffusion and cleaning the plurality of absorptive regions of the biochemical analysis unit therewith. Therefore, even in the case where a ligand or a receptor which should not be associated with the receptors or the ligands fixed in the plurality of absorptive regions of the biochemical analysis unit has been bonded therewith in the course of the receptor-ligand association reaction, it is possible to effectively peel off and remove the ligand or the receptor which should not be associated with the receptors or the ligands fixed in the plurality of absorptive regions of the biochemical analysis unit from the plurality of absorptive regions, whereby the ligand or the receptor which is to be associated with the receptors or the ligands fixed in the individual absorptive regions of the biochemical analysis unit can be associated therewith in a desired manner.

Further, according to this preferred aspect of the present invention, since the cleaning can be effected within micro-regions by determining the size of the at least one fluid passage for leading a solution to the plurality of absorptive regions formed in the biochemical analysis unit to be spaced apart from each other sufficiently small, the reaction can be facilitated in accordance with the principle of a micro-reactor and, therefore, the efficiency of the cleaning operation can be markedly improved.

In a preferred aspect of the present invention, the at least one fluid passage is formed so as to cut through the plurality of absorptive regions formed in the biochemical analysis unit to be spaced apart from each other.

According to this preferred aspect of the present invention, since the at least one fluid passage is formed so as to cut through the plurality of absorptive regions formed in the biochemical analysis unit to be spaced apart from each other, a reaction solution containing a ligand or a receptor can be fed to the plurality of absorptive regions formed in the biochemical analysis unit to be spaced from each other so as to cut through the plurality of absorptive regions, thereby selectively associating the ligand or the receptor contained in the reaction solution with the receptor or the ligand fixed in the plurality of absorptive regions of the biochemical analysis unit. Therefore, since it is possible to markedly increase the moving rate of the ligand or the receptor in comparison with the case of moving a ligand or a receptor by convection or diffusion and associating it with the receptor or the ligand fixed in the absorptive regions of the biochemical analysis unit, it is possible to markedly increase the reaction rate of association of the receptors or the ligands fixed in the plurality of absorptive regions of the biochemical analysis unit and the ligand or the receptor contained in the reaction solution and to markedly increase the possibility of association of the ligand or the receptor contained in the reaction solution with the receptors or the ligands fixed in deep portions of the plurality of absorptive regions of the biochemical analysis unit. Therefore, the ligand or the receptor contained in the reaction solution can be associated with the receptors or the ligands fixed in the plurality of absorptive regions of the biochemical analysis unit in a desired manner.

Furthermore, according to this preferred aspect of the present invention, since the at least one fluid passage is formed so as to cut through the plurality of absorptive regions formed in the biochemical analysis unit to be spaced apart from each other, a cleaning solution can be fed to the plurality of absorptive regions of the biochemical analysis unit so as to cut through the plurality of absorptive regions, thereby cleaning the plurality of absorptive regions of the biochemical analysis unit therewith. Therefore, since it is possible to efficiently clean the plurality of absorptive regions of the biochemical analysis unit with the cleaning solution in comparison with the case of moving the cleaning solution by convection or diffusion and cleaning the plurality of absorptive regions of the biochemical analysis unit therewith, even in the case where a ligand or a receptor which should not be associated with the receptors or the ligands fixed in the plurality of absorptive regions of the biochemical analysis unit has been bonded therewith in the course of the receptor-ligand association reaction, it is possible to effectively peel off and remove the ligand or the receptor which should not be associated with the receptors or the ligands fixed in the plurality of absorptive regions of the biochemical analysis unit from the plurality of absorptive regions. Therefore, since the ligand or the receptor which is to be associated with the receptors or the ligands fixed in the individual absorptive regions of the biochemical analysis unit can be associated therewith in a desired manner, it is possible to effectively prevent noise from being generated in biochemical analysis data and to produce biochemical analysis data having an excellent high quantitative characteristic with excellent repeatability.

In a preferred aspect of the present invention, a plurality of fluid passages are formed in the cartridge.

In a preferred aspect of the present invention, the plurality of absorptive regions are two-dimensionally formed in the biochemical analysis unit to be spaced apart from each other and a fluid passage is formed in the cartridge for each line of the plurality of absorptive regions so that the fluid passage can successively lead a solution to the plurality of absorptive regions constituting each line.

In a preferred aspect of the present invention, the fluid passage formed for each line of the plurality of absorptive regions is formed so as to sequentially cross the absorptive regions constituting the line.

In another preferred aspect of the present invention, the plurality of fluid passages are formed in the cartridge so as to be disposed on one side of the biochemical analysis unit held in the cartridge.

In a preferred aspect of the present invention, the plurality of absorptive regions are two-dimensionally formed in the biochemical analysis unit to be spaced apart from each other and a fluid passage is formed in the cartridge for each of the absorptive regions so as to feed a solution thereto.

In a further preferred aspect of the present invention, the fluid passage formed for each of the absorptive regions is formed so as to cut through the corresponding absorptive region.

In another preferred aspect of the present invention, the fluid passage formed for each of the absorptive regions is disposed on one side of the biochemical analysis unit held in the cartridge.

In the present invention, in the case where the at least one fluid passage or each of the fluid passages is formed in the cartridge so as to cut through the absorptive region(s) of the biochemical analysis unit, it is preferable for a portion of the fluid passage facing the absorptive region of the biochemical analysis unit to have a cross sectional area of 0.2 mm² or less, more preferably, 0.07 mm² or less.

In the case where the at least one fluid passage or each of the fluid passages is formed in the cartridge so as to cut through the absorptive region(s) of the biochemical analysis unit, if the portion of the fluid passage facing the absorptive region of the biochemical analysis unit has a cross sectional area of 0.2 mm² or less, since the reaction occurs within a micro-area, the reaction can be facilitated in accordance with the principle of a micro-reactor and, therefore, the efficiency of the receptor-ligand association reaction and the efficiency of the cleaning operation can be markedly improved. To the contrary, it is not preferable for a cross sectional area of the portion of the fluid passage facing the absorptive region of the biochemical analysis unit to exceed 0.2 mm² because the reaction cannot be sufficiently facilitated based on the principle of a micro-reactor.

In the present invention, in the case where the at least one fluid passage or each of the fluid passages is formed in the cartridge on one side of the biochemical analysis unit held in the cartridge, it is preferable for a portion of the fluid passage facing the absorptive region of the biochemical analysis unit to have a length of 0.5 mm or less, more preferably, 0.1 mm or less.

In the case where the at least one fluid passage or each of the fluid passages is formed in the cartridge on one side of the biochemical analysis unit held in the cartridge, if the length of the portion of the fluid passage facing the absorptive region of the biochemical analysis unit, namely, a passage length, is 0.5 mm or less, since the reaction occurs within a micro-area, the reaction can be facilitated in accordance with the principle of a micro-reactor and, therefore, the efficiency of the receptor-ligand association reaction and the efficiency of the cleaning operation can be markedly improved. To the contrary, it is not preferable for the length of the portion of the fluid passage facing the absorptive region of the biochemical analysis unit, namely, the passage length, to exceed 0.5 mm because the reaction cannot be sufficiently facilitated based on the principle of a micro-reactor.

In a preferred aspect of the present invention, specific binding substances whose structure or characteristics are known are fixed in the plurality of absorptive regions formed in the substrate of the biochemical analysis unit.

In a further preferred aspect of the present invention, the reaction solution contains, as a ligand or a receptor, a substance derived from a living organism and labeled with at least one kind of a labeling substance selected from a group consisting of a radioactive labeling substance, a fluorescent substance, a labeling substance which generates chemiluminescence emission when it contacts a chemiluminescent substrate and hapten and biochemical analysis data are recorded in the biochemical analysis unit by selectively hybridizing the substance derived from a living organism and labeled with at least one kind of the labeling substance with the specific binding substances fixed in the plurality of absorptive regions formed in the substrate of the biochemical analysis unit.

In a preferred aspect of the present invention, specific binding substances whose structure or characteristics are known are fixed in the plurality of absorptive regions formed in the substrate of the biochemical analysis unit and a substance derived from a living organism and labeled with hapten is selectively hybridized with the specific binding substances.

In a further preferred aspect of the present invention, the reaction solution contains, as a ligand or a receptor, an antibody for the hapten labeled with an enzyme which generates chemiluminescence emission when it contacts a chemiluminescent substrate or an enzyme which generates a fluorescent substance when it contacts a fluorescent substrate and biochemical analysis data are recorded in the biochemical analysis unit by binding the antibody for the hapten labeled with the enzyme with the hapten labeling the substance derived from a living organism and selectively hybridized with the specific binding substances fixed in the plurality of absorptive regions formed in the substrate of the biochemical analysis unit by an antigen-antibody reaction.

In the present invention, illustrative examples of the combination of hapten and antibody include digoxigenin and anti-digoxigenin antibody, theophylline and anti-theophylline antibody, fluorosein and anti-fluorosein antibody, and the like. Further, the combination of biotin and avidin, antigen and antibody may be utilized instead of the combination of hapten and antibody.

In a preferred aspect of the present invention, the substrate of the biochemical analysis unit is formed with a plurality of holes to be spaced apart from each other and the plurality of absorptive regions of the biochemical analysis unit are formed by charging an absorptive material in the plurality of holes formed in the substrate.

According to this preferred aspect of the present invention, since the substrate of the biochemical analysis unit is formed with a plurality of holes to be spaced apart from each other and the plurality of absorptive regions of the biochemical analysis unit are formed by charging an absorptive material in the plurality of holes formed in the substrate, it is possible to record biochemical analysis data having an excellent quantitative characteristic in the biochemical analysis unit by forcibly and uniformly feeding a reaction solution containing a ligand or a receptor labeled with a labeling substance to the plurality of absorptive regions of the biochemical analysis unit and selectively associating the ligand or the receptor contained in the reaction solution with the receptors or ligands fixed in the plurality of absorptive regions of the biochemical analysis unit.

In a further preferred aspect of the present invention, the substrate of the biochemical analysis unit is formed with a plurality of recesses to be spaced apart from each other and the plurality of absorptive regions of the biochemical analysis unit are formed by charging an absorptive material in the plurality of recesses formed in the substrate.

In another preferred aspect of the present invention, the substrate of the biochemical analysis unit is formed with a plurality of through-holes to be spaced apart from each other and the plurality of absorptive regions of the biochemical analysis unit are formed by charging an absorptive material in the plurality of through-holes formed in the substrate.

According to this preferred aspect of the present invention, since the substrate of the biochemical analysis unit is formed with a plurality of through-holes to be spaced apart from each other and the plurality of absorptive regions of the biochemical analysis unit are formed by charging an absorptive material in the plurality of through-holes formed in the substrate, a reaction solution containing a ligand or a receptor labeled with a labeling substance can be fed to the plurality of absorptive regions formed in the biochemical analysis unit so as to cut through the plurality of absorptive regions, thereby selectively associating the ligand or the receptor contained in the reaction solution with the receptor or the ligand fixed in the plurality of absorptive regions of the biochemical analysis unit. Therefore, since it is possible to markedly increase the moving rate of the ligand or the receptor in comparison with the case of moving a ligand or a receptor by convection or diffusion and associating it with the receptor or the ligand fixed in the absorptive regions of the biochemical analysis unit, it is possible to markedly increase the reaction rate of association of the receptors or the ligands fixed in the plurality of absorptive regions of the biochemical analysis unit and the ligand or the receptor contained in the reaction solution and to markedly increase the possibility of association of the ligand or the receptor contained in the reaction solution with the receptors or the ligands fixed in deep portions of the plurality of absorptive regions of the biochemical analysis unit. Therefore, the ligand or the receptor contained in the reaction solution can be associated with the receptors or the ligands fixed in the plurality of absorptive regions of the biochemical analysis unit in a desired manner and it is possible to record biochemical analysis data having an excellent quantitative characteristic in the biochemical analysis unit.

In a further preferred aspect of the present invention, the substrate of the biochemical analysis unit is formed with a plurality of through-holes to be spaced apart from each other and the plurality of absorptive regions of the biochemical analysis unit are formed by pressing an absorptive membrane containing an absorptive material into the plurality of through-holes formed in the substrate.

In another preferred aspect of the present invention, the biochemical analysis unit includes an absorptive substrate formed of an absorptive material and the plurality of absorptive regions of the biochemical analysis unit are formed by fixing a receptor or a ligand in regions of the absorptive substrate spaced apart from each other.

In a preferred aspect of the present invention, the substrate of the biochemical analysis unit has a property of attenuating radiation energy.

According to this preferred aspect of the present invention, even in the case of forming the plurality of absorptive regions in the substrate of the biochemical analysis unit at a high density, absorbing specific binding substances which can specifically bind a substance derived from a living organism and whose sequence, base length, composition and the like are known in the plurality of absorptive regions, hybridizing a substance derived from a living organism and labeled with a radioactive labeling substance and selectively labeling the plurality of absorptive regions with the radioactive labeling substance, when the biochemical analysis unit and a stimulable phosphor sheet formed with a stimulable phosphor layer to expose the stimulable phosphor layer formed in the stimulable phosphor sheet to the radioactive labeling substance selectively contained in the plurality of absorptive regions of the biochemical analysis unit, since the substrate of the biochemical analysis unit has a property of attenuating radiation energy, electron beams, (B rays) released from the radioactive labeling substance contained in the individual absorptive regions of the biochemical analysis unit can be effectively prevented from scattering in the substrate of the biochemical analysis unit. Therefore, it is possible to cause electron beams (B rays) to selectively enter a corresponding region of the stimulable phosphor layer to expose only the corresponding regions of the stimulable phosphor layer thereto, it is possible to produce biochemical analysis data having an excellent quantitative characteristic with high resolution by scanning the plurality of thus exposed stimulable phosphor layer regions with a stimulating ray and photoelectrically detecting stimulated emission released from the plurality of stimulable phosphor layer regions.

In a preferred aspect of the present invention, the substrate of the biochemical analysis unit has a property of reducing the energy of radiation to ⅕ or less when the radiation travels in the substrate by a distance equal to that between neighboring absorptive regions.

In a further preferred aspect of the present invention, the substrate of the biochemical analysis unit has a property of reducing the energy of radiation to 1/10 or less when the radiation travels in the substrate by a distance equal to that between neighboring absorptive regions.

In a further preferred aspect of the present invention, the substrate of the biochemical analysis unit has a property of reducing the energy of radiation to 1/50 or less when the radiation travels in the substrate by a distance equal to that between neighboring absorptive regions.

In a further preferred aspect of the present invention, the substrate of the biochemical analysis unit has a property of reducing the energy of radiation to 1/100 or less when the radiation travels in the substrate by a distance equal to that between neighboring absorptive regions.

In a further preferred aspect of the present invention, the substrate of the biochemical analysis unit has a property of reducing the energy of radiation to 1/500 or less when the radiation travels in the substrate by a distance equal to that between neighboring absorptive regions.

In a further preferred aspect of the present invention, the substrate of the biochemical analysis unit has a property of reducing the energy of radiation to 1/1,000 or less when the radiation travels in the substrate by a distance equal to that between neighboring absorptive regions.

In a preferred aspect of the present invention, the substrate of the biochemical analysis unit has a property of attenuating light energy.

According to this preferred aspect of the present invention, even in the case of forming the plurality of absorptive regions in the substrate of the biochemical analysis unit at a high density, absorbing specific binding substances which can specifically bind a substance derived from a living organism and whose sequence, base length, composition and the like are known in the plurality of absorptive regions, hybridizing a substance derived from a living organism and labeled with a fluorescent substance, thereby selectively labeling the plurality of absorptive regions with the fluorescent substance, irradiating the plurality of absorptive regions of the biochemical analysis unit with a stimulating ray, thereby exciting the fluorescent substance selectively contained in the plurality of absorptive regions and photoelectrically detecting fluorescence emission released from the plurality of absorptive region to produce biochemical analysis data, since the substrate of the biochemical analysis unit has a property of attenuating radiation energy, fluorescence emission released from the individual absorptive regions can be effectively prevented from scattering in the substrate of the biochemical analysis unit and mixing with fluorescence emission released from neighboring absorptive regions. Therefore, it is possible to produce biochemical analysis data with a high quantitative characteristic by photoelectrically detecting fluorescence emission.

Further, according to this preferred aspect of the present invention, even in the case of forming the plurality of absorptive regions in the substrate of the biochemical analysis unit at a high density, absorbing specific binding substances which can specifically bind a substance derived from a living organism and whose sequence, base length, composition and the like are known in the plurality of absorptive regions, hybridizing a substance derived from a living organism and labeled with a labeling substance which generates chemiluminescence emission when it contacts a chemiluminescent substrate, thereby selectively labeling the plurality of absorptive regions with the labeling substance which generates chemiluminescence emission when it contacts a chemiluminescent substrate, bringing the plurality of absorptive regions of the biochemical analysis unit into contact with a chemiluminescent substrate, thereby causing them to selectively release chemiluminescence emission and photoelectrically detecting chemiluminescence emission released from the plurality of absorptive regions of the biochemical analysis unit to produce biochemical analysis data, since the substrate of the biochemical analysis unit has a property of attenuating radiation energy, chemiluminescence emission released from the plurality of absorptive regions of the biochemical analysis unit can be effectively prevented from scattering in the substrate of the biochemical analysis unit and mixing with chemiluminescence emission released from neighboring absorptive regions. Therefore, it is possible to produce biochemical analysis data with a high quantitative characteristic by photoelectrically detecting chemiluminescence emission.

Furthermore, according to this preferred aspect of the present invention, even in the case of forming the plurality of absorptive regions in the substrate of the biochemical analysis unit at a high density, absorbing specific binding substances which can specifically bind a substance derived from a living organism and whose sequence, base length, composition and the like are known in the plurality of absorptive regions, hybridizing a substance derived from a living organism and labeled with a labeling substance which generates chemiluminescence emission when it contacts a chemiluminescent substrate, thereby recording chemiluminescence data in the plurality of absorptive regions of the biochemical analysis unit, bringing the plurality of absorptive regions of the biochemical analysis unit into contact with a chemiluminescent substrate, thereby causing them to selectively release chemiluminescence emission, superposing the biochemical analysis unit releasing chemiluminescence emission and a stimulable phosphor sheet formed with a stimulable phosphor layer, thereby exposing the stimulable phosphor layer formed in the stimulable phosphor sheet to chemiluminescence emission selectively released from the plurality of absorptive regions of the biochemical analysis unit and transferring chemiluminescence data to the stimulable phosphor layer, since the substrate of the biochemical analysis unit has a property of attenuating radiation energy, chemiluminescence emission released from the plurality of absorptive regions of the biochemical analysis unit can be effectively prevented from scattering in the substrate of the biochemical analysis unit. Therefore, it is possible to cause chemiluminescence emission selectively released from the plurality of absorptive regions of the biochemical analysis unit to selectively enter a corresponding region of the stimulable phosphor layer to expose only the corresponding regions of the stimulable phosphor layer thereto, it is possible to produce biochemical analysis data having an excellent quantitative characteristic with high resolution by scanning the plurality of thus exposed stimulable phosphor layer regions with a stimulating ray and photoelectrically detecting stimulated emission released from the plurality of stimulable phosphor layer regions.

In a preferred aspect of the present invention, the substrate of the biochemical analysis unit has a property of reducing the energy of light to ⅕ or less when the light travels in the substrate by a distance equal to that between neighboring absorptive regions.

In a further preferred aspect of the present invention, the substrate of the biochemical analysis unit has a property of reducing the energy of light to 1/10 or less when the light travels in the substrate by a distance equal to that between neighboring absorptive regions.

In a further preferred aspect of the present invention, the substrate of the biochemical analysis unit has a property of reducing the energy of light to 1/50 or less when the light travels in the substrate by a distance equal to that between neighboring absorptive regions.

In a further preferred aspect of the present invention, the substrate of the biochemical analysis unit has a property of reducing the energy of light to 1/100 or less when the light travels in the substrate by a distance equal to that between neighboring absorptive regions.

In a further preferred aspect of the present invention, the substrate of the biochemical analysis unit has a property of reducing the energy of light to 1/500 or less when the light travels in the substrate by a distance equal to that between neighboring absorptive regions.

In a further preferred aspect of the present invention, the substrate of the biochemical analysis unit has a property of reducing the energy of light to 1/1,000 or less when the light travels in the substrate by a distance equal to that between neighboring absorptive regions.

In a preferred aspect of the present invention, the substrate of the biochemical analysis unit is formed with 10 or more absorptive regions.

In a further preferred aspect of the present invention, the substrate of the biochemical analysis unit is formed with 50 or more absorptive regions.

In a further preferred aspect of the present invention, the substrate of the biochemical analysis unit is formed with 100 or more absorptive regions.

In a further preferred aspect of the present invention, the substrate of the biochemical analysis unit is formed with 500 or more absorptive regions.

In a further preferred aspect of the present invention, the substrate of the biochemical analysis unit is formed with 1,000 or more absorptive regions.

In a further preferred aspect of the present invention, the substrate of the biochemical analysis unit is formed with 5,000 or more absorptive regions.

In a further preferred aspect of the present invention, the substrate of the biochemical analysis unit is formed with 10,000 or more absorptive regions.

In a further preferred aspect of the present invention, the substrate of the biochemical analysis unit is formed with 50,000 or more absorptive regions.

In a further preferred aspect of the present invention, the substrate of the biochemical analysis unit is formed with 100,000 or more absorptive regions.

In a preferred aspect of the present invention, each of the plurality of absorptive regions formed in the substrate of the biochemical analysis unit has a size of less than 5 mm².

In a further preferred aspect of the present invention, each of the plurality of absorptive regions formed in the substrate of the biochemical analysis unit has a size of less than 1 mm².

In a further preferred aspect of the present invention, each of the plurality of absorptive regions formed in the substrate of the biochemical analysis unit has a size of less than 0.5 mm².

In a further preferred aspect of the present invention, each of the plurality of absorptive regions formed in the substrate of the biochemical analysis unit has a size of less than 0.1 mm².

In a further preferred aspect of the present invention, each of the plurality of absorptive regions formed in the substrate of the biochemical analysis unit has a size of less than 0.05 mm².

In a further preferred aspect of the present invention, each of the plurality of absorptive regions formed in the substrate of the biochemical analysis unit has a size of less than 0.01 mm².

In a preferred aspect of the present invention, the plurality of absorptive regions are formed in the substrate of the biochemical analysis unit at a density of 10 or more per cm².

In a further preferred aspect of the present invention, the plurality of absorptive regions are formed in the substrate of the biochemical analysis unit at a density of 50 or more per cm².

In a further preferred aspect of the present invention, the plurality of absorptive regions are formed in the substrate of the biochemical analysis unit at a density of 100 or more per cm².

In a further preferred aspect of the present invention, the plurality of absorptive regions are formed in the substrate of the biochemical analysis unit at a density of 500 or more per cm².

In a further preferred aspect of the present invention, the plurality of absorptive regions are formed in the substrate of the biochemical analysis unit at a density of 1,000 or more per cm².

In a further preferred aspect of the present invention, the plurality of absorptive regions are formed in the substrate of the biochemical analysis unit at a density of 5,000 or more per cm².

In a further preferred aspect of the present invention, the plurality of absorptive regions are formed in the substrate of the biochemical analysis unit at a density of 10,000 or more per cm².

In a further preferred aspect of the present invention, the plurality of absorptive regions are formed in the substrate of the biochemical analysis unit at a density of 50,000 or more per cm².

In a further preferred aspect of the present invention, the plurality of absorptive regions are formed in the substrate of the biochemical analysis unit at a density of 100,000 or more per cm².

In a preferred aspect of the present invention, the plurality of absorptive regions are formed in the substrate of the biochemical analysis unit in a regular pattern.

In a preferred aspect of the present invention, each of the plurality of absorptive regions is formed substantially circular in the substrate of the biochemical analysis unit in a regular pattern.

In-another preferred aspect of the present invention, each of the plurality of absorptive regions is formed substantially rectangular in a regular pattern.

In the present invention, the material for forming the substrate of the biochemical analysis unit is preferably capable of attenuating radiation energy and/or light energy but is not particularly limited. The material for forming the substrate of the biochemical analysis unit may be any type of inorganic compound material or organic compound material and the substrate of the biochemical analysis unit can preferably be formed of a metal material, a ceramic material or a plastic material.

Illustrative examples of inorganic compound materials preferably usable for forming the substrate of the biochemical analysis unit in the present invention include metals such as gold, silver, copper, zinc, aluminum, titanium, tantalum, chromium, iron, nickel, cobalt, lead, tin, selenium and the like; alloys such as brass, stainless steel, bronze and the like; silicon materials such as silicon, amorphous silicon, glass, quartz, silicon carbide, silicon nitride and the like; metal oxides such as aluminum oxide, magnesium oxide, zirconium oxide and the like; and inorganic salts such as tungsten carbide, calcium carbide, calcium sulfate, hydroxy apatite, gallium arsenide and the like. These may have either a monocrystal structure or a polycrystal sintered structure such as amorphous, ceramic or the like.

In the present invention, a high molecular compound can preferably be used as an organic compound material preferably usable for forming the substrate of the biochemical analysis unit. Illustrative examples of high molecular compounds preferably usable for forming the substrate of the biochemical analysis unit in the present invention include polyolefins such as polyethylene, polypropylene and the like; acrylic resins such as polymethyl methacrylate, polybutylacrylate/polymethyl methacrylate copolymer and the like; polyacrylonitrile; polyvinyl chloride; polyvinylidene chloride; polyvinylidene fluoride; polytetrafluoroethylene; polychlorotrifuluoroethylene; polycarbonate; polyesters such as polyethylene naphthalate, polyethylene terephthalate and the like; nylons such as nylon-6, nylon-6,6, nylon-4,10 and the like; polyimide; polysulfone; polyphenylene sulfide; silicon resins such as polydiphenyl siloxane and the like; phenol resins such as novolac and the like; epoxy resin; polyurethane; polystyrene, butadiene-styrene copolymer; polysaccharides such as cellulose, acetyl cellulose, nitrocellulose, starch, calcium alginate, hydroxypropyl methyl cellulose and the like; chitin; chitosan; urushi (Japanese lacquer); polyamides such as gelatin, collagen, keratin and the like; and copolymers of these high molecular materials. These may be a composite compound, and metal oxide particles, glass fiber or the like may be added thereto as occasion demands. Further, an organic compound material may be blended therewith.

Since the capability of attenuating radiation energy generally increases as specific gravity increases, the substrate of the biochemical analysis unit is preferably formed of a compound material or a composite material having specific gravity of 1.0 g/cm³ or more and more preferably formed of a compound material or a composite material having specific gravity of 1.5 g/cm³ to 23 g/cm³.

Further, since the capability of attenuating light energy generally increases as scattering and/or absorption of light increases, the substrate of the biochemical analysis unit preferably has absorbance of 0.3 per cm (thickness) or more and more preferably has absorbance of 1 per cm (thickness) or more. The absorbance can be determined by placing an integrating sphere immediately behind a plate-like member having a thickness of T cm, measuring an amount A of transmitted light at a wavelength of probe light or emission light used for measurement by a spectrophotometer, and calculating A/T. In the present invention, a light scattering substance or a light absorbing substance may be added to the substrate of the biochemical analysis unit in order to improve the capability of attenuating light energy. Particles of a material different from a material forming the substrate of the biochemical analysis unit may be preferably used as a light scattering substance and a pigment or dye may be preferably used as a light absorbing substance.

In the present invention, a porous material or a fiber material may be preferably used as the absorptive material for forming the absorptive regions of the biochemical analysis unit. The absorptive regions may be formed by combining a porous material and a fiber material.

In the present invention, a porous material for forming the absorptive regions of the biochemical analysis unit may be any type of an organic material or an inorganic material and may be an organic/inorganic composite material.

In the present invention, an organic porous material used for forming the absorptive regions of the biochemical analysis unit is not particularly limited but a carbon porous material such as an activated carbon or a porous material capable of forming a membrane filter is preferably used. Illustrative examples of porous materials capable of forming a membrane filter include nylons such as nylon-6, nylon-6,6, nylon-4,10; cellulose derivatives such as nitrocellulose, acetyl cellulose, butyric-acetyl cellulose; collagen; alginic acids such as alginic acid, calcium alginate, alginic acid/poly-L-lysine polyionic complex; polyolefins such as polyethylene, polypropylene; polyvinyl chloride; polyvinylidene chloride; polyfluoride such as polyvinylidene fluoride, polytetrafluoride; and copolymers or composite materials thereof

In the present invention, an inorganic porous material used for forming the absorptive regions of the biochemical analysis unit is not particularly limited. Illustrative examples of inorganic porous materials preferably usable in the present invention include metals such as platinum, gold, iron, silver, nickel, aluminum and the like; metal oxides such as alumina, silica, titania, zeolite and the like; metal salts such as hydroxy apatite, calcium sulfate and the like; and composite materials thereof.

In the present invention, a fiber material used for forming the absorptive regions of the biochemical analysis unit is not particularly limited. Illustrative examples of fiber materials preferably usable in the present invention include nylons such as nylon-6, nylon-6,6, nylon-4,10; and cellulose derivatives such as nitrocellulose, acetyl cellulose, butyric-acetyl cellulose.

In the present invention, the absorptive layer of the biochemical analysis unit may be formed using an oxidization process such as an electrolytic process, a plasma process, an arc discharge process or the like; a primer process using a silane coupling agent, titanium coupling agent or the like; and a surface-active agent process or the like.

The above and other objects and features of the present invention will become apparent from the following description made with reference to the accompanying drawings.

### BRIEF DESCRIPTION OF THE DRAWINGS

FIG. 1 is a schematic perspective view showing a biochemical analysis unit used in a method for recording biochemical analysis data which is a preferred embodiment of the present invention.

FIG. 2 is a schematic front view showing a spotting device.

FIG. 3 is a schematic longitudinal center cross sectional view showing a cartridge for a biochemical analysis unit which is a preferred embodiment of the present invention.

FIG. 4 is a schematic cross sectional view taken along a line A-A in FIG. 3.

FIG. 5 is a schematic cross sectional view taken along a line B-B in FIG. 3.

FIG. 6 is a schematic longitudinal cross sectional view showing an apparatus for a receptor-ligand association reaction which is a preferred embodiment of the present invention.

FIG. 7 is a schematic perspective view showing a stimulable phosphor sheet onto which radiation data are to be transferred.

FIG. 8 is a schematic cross-sectional view showing a method for exposing a number of the stimulable phosphor layer regions formed in the stimulable phosphor sheet by a radioactive labeling substance contained in a number of the absorptive regions formed in the biochemical analysis unit.

FIG. 9 is a schematic view showing a scanner for reading radiation data recorded in a number of the stimulable phosphor layer regions formed in the support of the stimulable phosphor sheet to produce biochemical analysis data.

FIG. 10 is a schematic perspective view showing details in the vicinity of a photomultiplier of a scanner shown in FIG. 9.

FIG. 11 is a schematic cross-sectional view taken along a line A-A in FIG. 10.

FIG. 12 is a schematic cross-sectional view taken along a line B-B in FIG. 10.

FIG. 13 is a schematic cross-sectional view taken along a line C-C in FIG. 10.

FIG. 14 is a schematic cross-sectional view taken along a line D-D in FIG. 10.

FIG. 15 is a schematic plan view showing the scanning mechanism of an optical head.

FIG. 16 is a block diagram of a control system, an input system, a drive system and a detection system of the scanner shown in FIG. 9.

FIG. 17 is a schematic perspective view showing a stimulable phosphor sheet onto which chemiluminescence data are to be transferred.

FIG. 18 is a schematic view showing a scanner for reading chemiluminescence data recorded in a number of stimulable phosphor layer regions formed in a support of a stimulable phosphor sheet and producing biochemical analysis data.

FIG. 19 is a schematic perspective view showing details in the vicinity of a photomultiplier of a scanner shown in FIG. 18.

FIG. 20 is a schematic cross-sectional view taken along a line E-E in FIG. 19.

FIG. 21 is a schematic perspective view showing a cartridge for a biochemical analysis unit which is another preferred embodiment of the present invention.

FIG. 22 is a schematic cross sectional view taken along a line C-C in FIG. 21.

FIG. 23 is a schematic cross sectional view taken along a line D-D in FIG. 21.

FIG. 24 is a schematic perspective view showing a cartridge for a biochemical analysis unit which is a further preferred embodiment of the present invention.

FIG. 25 is a schematic cross sectional view taken along a line E-E in FIG. 24.

FIG. 26 is a schematic cross sectional view taken along a line F-F in FIG. 24.

### DESCRIPTION OF THE PREFERRED EMBODIMENTS

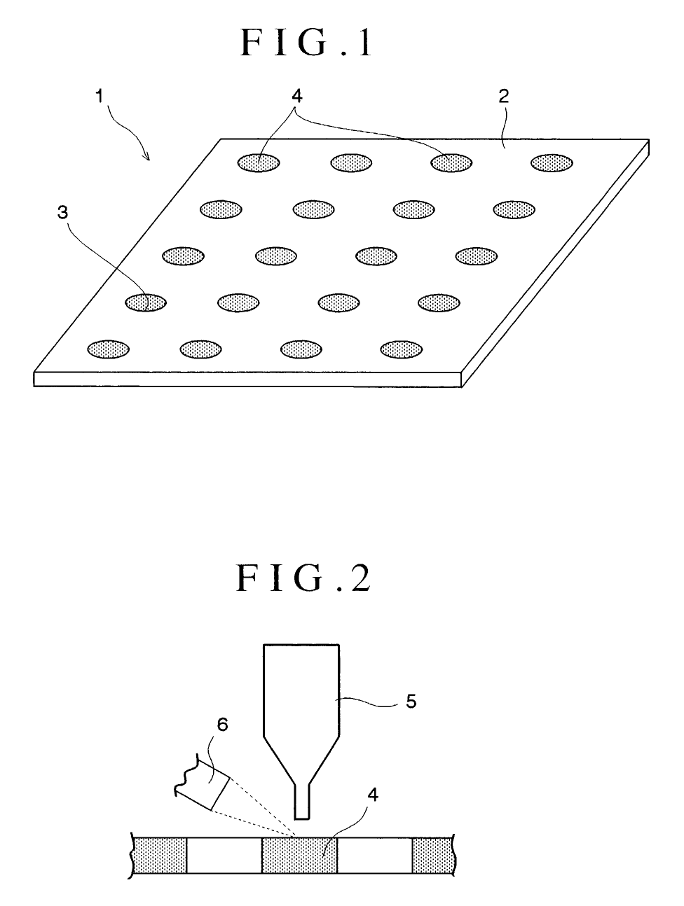

FIG. 1 is a schematic perspective view showing a biochemical analysis unit used in a method for recording biochemical analysis data which is a preferred embodiment of the present invention.

As shown in FIG. 1, a biochemical analysis unit 1 according to this embodiment includes a substrate 2 formed of stainless steel and formed with a number of substantially circular through-holes 3 at a high density, and a number of absorptive regions 4 are dot-like formed by charging nylon-6 in the through-holes 3.

Although not accurately shown in FIG. 1, in this embodiment, about 10,000 through-holes 3 having a size of about 0.01 mm² are regularly formed at a density of about 5,000 per cm² in the substrate 2.

A number of absorptive regions 4 are formed by charging nylon-6 in the through-holes 3 formed in the substrate in such a manner that the surfaces of the absorptive regions 4 are located at the same height level as that of the substrate 2.

FIG. 2 is a schematic front view showing a spotting device.

As shown in FIG. 2, when biochemical analysis is performed, a solution containing specific binding substances such as a plurality of cDNAs whose sequences are known but differ from each other are spotted using a spotting device 5 onto a number of the absorptive regions 4 of the biochemical analysis unit 1 and the specific binding substances are fixed therein.

As shown in FIG. 2, the spotting device includes an injector 5 for ejecting a solution of specific binding substances toward the biochemical analysis unit 1 and a CCD camera 6 and is constituted so that the solution of specific binding substances such as cDNAs are spotted from the injector 6 when the tip end portion of the injector 5 and the center of the absorptive region 4 into which the solution containing specific binding substances is to be spotted are determined to coincide with each other as a result of viewing them using the CCD camera 6, thereby ensuring that the solution of specific binding substances can be accurately spotted into a number of the absorptive regions 4 of the biochemical analysis unit 1.

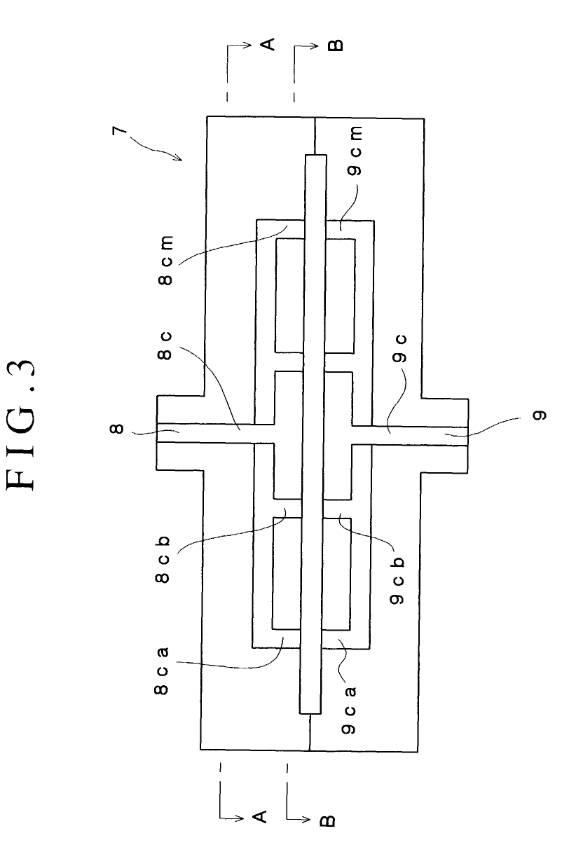

FIG. 3 is a schematic longitudinal center cross sectional view showing a cartridge for a biochemical analysis unit which is a preferred embodiment of the present invention.

As shown in FIG. 3, the cartridge 7 for a biochemical analysis unit according to this embodiment includes an upper half portion 7A and a lower half portion 7B and the biochemical analysis unit 1 is held between the upper half portion 7A and the lower half portion 7B.

A solution feed passage 8 is formed at the substantial center portion of the upper half portion 7A for feeding a solution into the cartridge 7 and a solution discharge passage 9 is formed at the substantial center portion of the lower half portion 7B for discharging a solution from the cartridge 7.

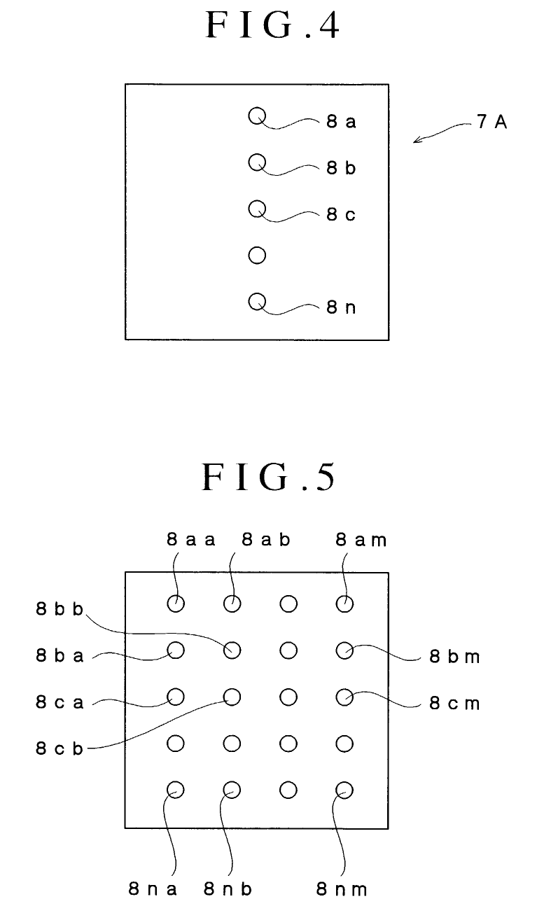

FIG. 4 is a schematic cross sectional view taken along a line A-A in FIG. 3.

As shown in FIG. 4, the solution feed passage 8 branches into n first branch passages 8a, 8b, 8c, 8d, . . . , 8n correspondingly to the number n of columns of the absorptive regions 4 formed in the substrate 2 of the biochemical analysis unit I in a direction perpendicular to the surface of the drawing sheet in FIG. 3.

FIG. 5 is a schematic cross sectional view taken along a line B-B in FIG. 3.

As shown in FIG. 5, each of the first branch passages 8a, 8b, 8c, 8d, . . . , 8n branches into m second branch passages 8aa, 8ab, . . . , 8am; 8ba, 8bb, . . . , 8bm; 8ca, 8cb, . . . , 8cm; . . . ; 8na, 8nb, . . . , 8nm correspondingly to the number m of rows of the absorptive regions 4 formed in the substrate 2 of the biochemical analysis unit 1 in a direction parallel with the surface of the drawing sheet of FIG. 3. In FIG. 3, only the second branch passages 8ca, 8cb, . . . , 8cm are shown.

Therefore, the second branch passages 8aa, 8ab, . . . , 8am; 8ba, 8bb, . . . , 8bm; 8ca, 8cb, . . . , 8cm; . . . ; 8na, 8nb, . . . , 8nm whose number is equal to the number of the absorptive regions 4 formed in the substrate 2 of the biochemical analysis unit 1 are formed and each of the second branch passages 8aa, 8ab, . . . , 8am; 8ba, 8bb, . . . , 8bm; 8ca, 8cb, . . . , 8cm; . . . ; 8na, 8nb, . . . , 8nm is formed at a position corresponding to one of the absorptive regions 4 of the biochemical analysis unit 1 held in the cartridge 7.

In this embodiment, each of the second branch passages 8aa, 8ab, . . . , 8am; 8ba, 8bb, . . . , 8bm; 8ca, 8cb, . . . , 8cm; . . . ; 8na, 8nb, . . . , 8nm is formed so that the cross section thereof has the same size as that of the absorptive region 4 formed in the substrate 2 of the biochemical analysis unit 1.

The lower half portion 7B is formed with third branch passages 9aa, 9ab, . . . , 9am; 9ba, 9bb, . . . , 9bm; 9ca, 9cb, . . . , 9cm; . . . ; 9na, 9nb, . . . , 9nm at positions facing the second branch passages 8aa, 8ab, . . . , 8am; 8ba, 8bb, . . . , 8bm; 8ca, 8cb, . . . , 8cm; . . . ; 8na, 8nb, . . . , 8nm via the absorptive regions 4 of the biochemical analysis unit 1 held in the cartridge 7 in the same pattern as that of the second branch passages 8aa, 8ab, . . . , 8am; 8ba, 8bb, . . . , 8bm; 8ca, 8cb, . . . , 8cm; . . . ; 8na, 8nb, . . . , 8nm shown in FIG. 5. In FIG. 3, only the third branch passages 9ca, 9cb, . . . , 9cm are shown.

In this embodiment, each of the third branch passages 9aa, 9ab, . . . , 9am; 9ba, 9bb, . . . , 9bm; 9ca, 9cb, . . . , 9cm; . . . ; 9na, 9nb, . . . , 9nm is formed so that the cross section thereof has the same size as that of the absorptive region 4 formed in the substrate 2 of the biochemical analysis unit 1.

Although not shown in the figures, the third branch passages 9aa, 9ab, . . . , 9am; 9ba, 9bb, . . . , 9bm; 9ca, 9cb, . . . , 9cm; . . . ; 9na, 9nb, . . . , 9nm merge into fourth branch passages 9a, 9b, 9c, . . . , 9n formed in the same pattern as that of the first branch passages 8a, 8b, 8c, 8d, . . . , 8n and the fourth branch passages 9a, 9b, 9c, . . . , 9n merge into the solution discharge passage 9.

Therefore, a solution fed into the cartridge 7 through the solution feed passage 8 is fed into the second branch passages 8aa, 8ab, . . . , 8am; 8ba, 8bb, . . . , 8bm; 8ca, 8cb, . . . , 8cm; . . . ; 8na, 8nb, . . . , 8nm via the first branch passages 8a, 8b, 8c, 8d, . . . , 8n, passes through the absorptive regions 4 of the biochemical analysis unit 1 that the second branch passages 8aa, 8ab, . . . , 8am; 8ba, 8bb, . . . , 8bm; 8ca, 8cb, . . . , 8cm; . . . ; 8na, 8nb, . . . , 8nm face, is led into the third branch passages 9aa, 9ab, . . . , 9am; 9ba, 9bb, . . . , 9bm; 9ca, 9cb, . . . , 9cm; . . . ; 9na, 9nb, . . . , 9nm, flows through the fourth branch passages 9a, 9b, 9c, . . . , 9n and is discharged to the outside of the cartridge 7 through the solution discharge passage 9.

The biochemical analysis unit 1 is accommodated and biochemical analysis data are recorded in a number of the absorptive regions 4 formed in the substrate 2 of the biochemical analysis unit 1 using an apparatus for a receptor-ligand association reaction.

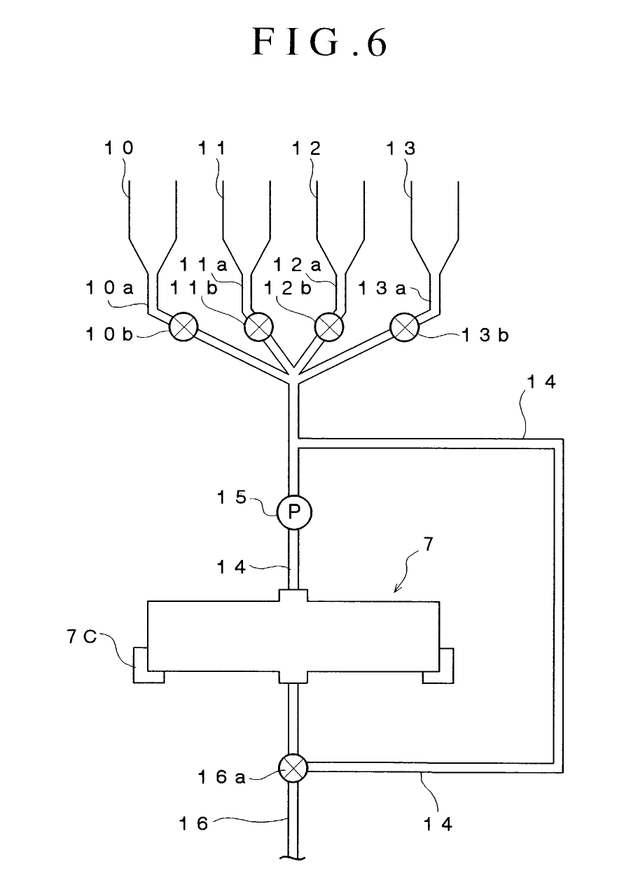

FIG. 6 is a schematic longitudinal cross sectional view showing an apparatus for a receptor-ligand association reaction which is a preferred embodiment of the present invention.

As shown in FIG. 6, the apparatus for a receptor-ligand association reaction according to this embodiment includes a support base 7C for supporting the cartridge 7 accommodating the biochemical analysis unit 1, a hybridization buffer tank 10 for accommodating a hybridization buffer, a probe solution chip 11 for accommodating a probe solution, an antibody solution tank 12 for accommodating an antibody solution, a cleaning solution tank 13 for accommodating a cleaning solution, a hybridization buffer feed pipe 10a through which a hybridization buffer is fed, a probe solution feed pipe 11a through which a probe solution is fed, an antibody solution feed pipe 12a through which an antibody solution is fed, a cleaning solution feed pipe 13a through which a cleaning solution is fed, a change-over valve 10b provided in the hybridization buffer feed pipe 10a, a change-over valve 11b provided in the probe solution feed pipe 11a, a change-over valve 12b provided in the antibody solution feed pipe 12a, a change-over valve 13b provided in the cleaning solution feed pipe 13a, a solution circulation pipe 14 connected to the cartridge 7 through which a hybridization buffer, a mixed solution of a hybridization buffer and a probe solution, an antibody solution or a cleaning solution flows, a pump 15 provided in the solution circulation pipe 14, a solution discharge pipe 16 for discharging a solution from the cartridge 7 and the solution circulation pipe 14, and a change-over valve 16a provided at a bifurcated portion of the solution circulation pipe 14 and the solution discharge pipe 16.

In this embodiment, the change-over valve 10b provided in the hybridization buffer feed pipe 10a is constituted as a three-way valve so that it can selectively assume a first position where the hybridization buffer feed pipe 10a and the solution circulation pipe 14 communicate with each other, a second position where the atmosphere and the solution circulation pipe 14 communicate with each other or a third position where communication between the hybridization buffer tank 10 and the atmosphere, and the solution circulation pipe 14 is shut off, and the change-over valve 11b provided in the probe solution feed pipe 11a is constituted as a three-way valve so that it can selectively assume a first position where the probe solution feed pipe 11a and the soluton circulation pipe 14 communicate with each other, a second position where the atmosphere and the solution circulation pipe 14 communicate with each other or a third position where communication between the probe solution tank 11 and the atmosphere, and the solution circulation pipe 14 is shut off.

Further, the change-over valve 12b provided in the antibody solution feed pipe 12a is constituted as a three-way valve so that it can selectively assume a first position where the antibody solution feed pipe 12a and the solution circulation pipe 14 communicate with each other, a second position where the atmosphere and the solution circulation pipe 14 communicates with each other or a third position where communication between the antibody solution tank 12 and the atmosphere, and the solution circulation pipe 14 is shut off, and the change-over valve 13b provided in the cleaning solution feed pipe 13a is constituted as a three-way valve so that it can selectively assume a first position where the cleaning solution feed pipe 13a and the solution circulation pipe 14 communicate with each other, a second position where the atmosphere and the solution circulation pipe 14 communicate with each other or a third position where communication between the cleaning solution feed pipe 13a and the atmosphere, and the solution circulation pipe 14 is shut off

Furthermore, the change-over valve 16a provided at the bifurcated portion of the solution circulation pipe 14 and the solution discharge pipe 16 is constituted as a two-way valve, which can assume a first position where the upstream portion of the solution circulation pipe 14 and the downstream portion of the solution circulation pipe 14 communicate with each other and a second position where the solution circulation pipe 14 and the solution discharge pipe 16 communicate with each other.

As shown in FIG. 6, when biochemical analysis data are to be recorded in a number of the absorptive regions 4 of the biochemical analysis unit 1 accommodated in the cartridge 7, one end portion of the solution circulation pipe 14 is connected to the solution feed passage 8 of the cartridge 7 for a biochemical analysis unit and the other end portion is connected to the solution discharge passage 9 of the cartridge 7.

In the thus constituted apparatus for receptor-legand association, a substance derived from a living body, labeled with a labeling substance and contained in a probe solution selectively hybridizes specific binding substances contained in a number of the absorptive regions 4 of the biochemical analysis unit 1 in the following manner.

A hybridization buffer is prepared and accommodated in the hybridization buffer tank 10.

Then, the change-over valve 11b provided in the probe solution feed pipe 11a, the change-over valve 12b provided in the antibody solution feed pipe 12a and the change-over valve 13b provided in the cleaning solution feed pipe 13a are located at their second positions where the atmosphere and the solution circulation pipe 14 communicate with each other and the change-over valve 16a provided at the bifurcated portion of the solution circulation pipe 14 and the solution discharge pipe 16 is located at its first position.

When the change-over valve 10b provided in the hybridization buffer feed pipe 10a has been located at its first position where the hybridization buffer feed pipe 10a and the solution circulation pipe 14 communicate with each other, the pump 15 is driven.

As a result, a hybridization buffer accommodated in the hybridization buffer tank 10 is fed into the cartridge 7 via the hybridization buffer feed pipe 10a and the solution circulation pipe 14.

Since the solution circulation pipe 14 is connected to the solution feed passage 8 of the cartridge 7 for a biochemical analysis unit, the hybridization buffer is fed into the cartridge 7 for a biochemical analysis unit through the solution feed passage 8.

In this embodiment, since the solution feed passage 8 branches into the n first branch passages 8a, 8b, 8c, . . . , 8n whose number is equal to the number of columns of the absorptive regions 4 of the biochemical analysis unit 1, the hybridization buffer flows into the n first branch passages 8a, 8b, 8c, . . . , 8n from the solution feed passage 8.

Since each of the first branch passages 8a, 8b, 8c, . . . , 8n branches into the m second branch passages 8aa, 8ab, . . . , 8am; 8ba, 8bb, . . . , 8bm; 8ca, 8cb, . . . , 8cm; . . . ; 8na, 8nb, . . . , 8nm which are formed at positions facing the absorptive regions 4 of the biochemical analysis unit 1 held in the cartridge 7 so as to correspond to the number m of rows of the absorptive regions 4 of the biochemical analysis unit 1 held in the cartridge 7, the hybridization buffer flowing into the first branch passages 8a, 8b, 8c, . . . , 8n further flows into the second branch passages 8aa, 8ab, . . . , 8am; 8ba, 8bb, . . . , 8bm; 8ca, 8cb, . . . , 8cm; . . . ; 8na, 8nb, . . . , 8nm from the first branch passages 8a, 8b, 8c, . . . , 8n and is fed to the individual absorptive regions 4 of the biochemical analysis unit 1 held in the cartridge 7.

The hybridization buffer fed to the individual absorptive regions 4 of the biochemical analysis unit 1 held in the cartridge 7 passes through the corresponding absorptive regions 4 and flows into the third branch passages 9aa, 9ab, . . . , 9am; 9ba, 9bb, . . . , 9bm; 9ca, 9cb, . . . , 9cm; . . . ; 9na, 9nb, . . . , 9nm at positions facing the second branch passages 8aa, 8ab, . . . , 8am; 8ba, 8bb, . . . , 8bm; 8ca, 8cb, . . . , 8cm; . . . ; 8na, 8nb, . . . , 8nm via the absorptive regions 4 of the biochemical analysis unit 1 held in the cartridge 7.

In this manner, pre-hybridization is performed.

Since the third branch passages 9aa, 9ab, . . . , 9am; 9ba, 9bb, . . . , 9bm; 9ca, 9cb, . . . , 9cm; . . . ; 9na, 9nb, . . . , 9nm merge into the fourth branch passages 9a, 9b, 9c, . . . , 9n formed in the same pattern as that of the first branch passages 8a, 8b, 8c, 8d, . . . , 8n, the hybridization buffer flows into the fourth branch passages 9a, 9b, 9c, . . . , 9n.

In this embodiment, since the fourth branch passages 9a, 9b, 9c, . . . , 9n merge to be connected to the solution discharge passage 9 and the solution circulation pipe 14 is connected to the solution discharge passage 9, the hybridization buffer is fed into the solution circulation pipe 14 via the fourth branch passages 9a, 9b, 9c, . . . , 9n and the solution discharge passage 9 of the cartridge 7 and recycled into the cartridge 7.

In this manner, when an inner space of the cartridge 7 and the solution circulation pipe 14 has been filled with the hybridization buffer, the change-over valve 10b provided in the hybridization buffer feed pipe 10a is located at its third position where communication between the hybridization buffer tank 10 and the atmosphere, and the solution circulation pipe 14 is shut off and the change-over valve 11b provided in the probe solution feed pipe 11a, the change-over valve 12b provided in the antibody solution feed pipe 12a and the change-over valve 13b provided in the cleaning solution feed pipe 13a are located at their third positions.

On the other hand, the pump 15 continues to be driven and as a result, the hybridization buffer filling the inner space of the cartridge 7 and the solution circulation pipe 14 is circulated through the cartridge 7 and the solution circulation pipe 14, whereby the hybridization buffer is forcibly fed so as to pass through a number of the absorptive regions 4 of the biochemical analysis unit 1 held in the cartridge 7.

When a first predetermined time period has passed, the pump 15 is stopped and pre-hybridization is completed.

Then, a probe solution is prepared and accommodated in the probe solution chip 11.

In the case where a specific binding substance such as cDNA is to be labeled with a radioactive labeling substance, a probe solution containing a substance derived from a living organism and labeled with a radioactive labeling substance as a probe is prepared and is accommodated in the probe solution chip 11.

On the other hand, in the case where a specific binding substance such as cDNA is to be labeled with a fluorescent substance, a probe solution containing a substance derived from a living organism and labeled with a fluorescent substance as a probe is prepared and is accommodated in the probe solution chip 11.

Further, in the case where a specific binding substance such as cDNA is to be labeled with a labeling substance which generates chemiluminescence emission when it contacts a chemiluminescent substrate, a probe solution containing a substance derived from a living organism and labeled with hapten such as digoxigenin as a probe is prepared and is accommodated in the probe solution chip 11.

It is possible to prepare a probe solution containing two or more substances derived from a living organism among a substance derived from a living organism and labeled with a radioactive labeling substance, a substance derived from a living organism and labeled with a fluorescent substance such as a fluorescent dye and a substance derived from a living organism and labeled with hapten such as digoxigenin and accommodate it in the probe solution chip 12b. In this embodiment, a probe solution containing a substance derived from a living organism and labeled with a radioactive labeling substance, a substance derived from a living organism and labeled with a fluorescent substance such as a fluorescent dye and a substance derived from a living organism and labeled with hapten such as digoxigenin is prepared and accommodated in the probe solution chip 11.

When the probe solution has been accommodated in the probe solution chip 11, the change-over valve 10b provided in the hybridization buffer feed pipe 10a, the change-over valve 12b provided in the antibody solution feed pipe 12a and the change-over valve 13b provided in the cleaning solution feed pipe 13a are located at their second positions where the atmosphere and the solution circulation pipe 14 communicate with each other and the change-over valve 16a provided at the bifurcated portion of the solution circulation pipe 14 and the solution discharge pipe 16 is located at its first position.

Then, the change-over valve 11b provided in the probe solution feed pipe 11a is located at its first position where the probe solution feed pipe 11a and the solution circulation pipe 14 communicate with each other and the pump 15 is driven.

As a result, a probe solution accommodated in the probe solution chip 11 is fed into the solution circulation pipe 14 via the probe solution feed pipe 11a and mixed with the hybridization buffer filling the inner space of the cartridge 7 and the solution circulation pipe 14.

When a predetermined amount of the probe solution has been fed from the probe solution chip 11, the change-over valve 11b provided in the probe solution feed pipe 11a is located at its third position where communication between the probe solution tank 11 and the atmosphere, and the solution circulation pipe 14 is shut off and the change-over valve 10b provided in the hybridization buffer feed pipe 10a, the change-over valve 12b provided in the antibody solution feed pipe 12a and the change-over valve 13b provided in the cleaning solution feed pipe 13a are located at their third positions.

On the other hand, the pump 15 continues to be driven and therefore, the mixed solution produced by mixing the probe solution with the hybridization buffer filling the inner space of the cartridge 7 and the solution circulation pipe 14 is fed into the cartridge 7 via the solution circulation pipe 14 and the solution feed passage 8.

Since the solution feed passage 8 branches into the n first branch passages 8a, 8b, 8c, . . . , 8n, the mixed solution of the hybridization buffer and the probe solution further flows into the n first branch passages 8a, 8b, 8c, . . . , 8n from the solution feed passage 8.

Since each of the first branch passages 8a, 8b, 8c, . . . , 8n branches into the m second branch passages 8aa, 8ab, . . . , 8am; 8ba, 8bb, . . . , 8bm; 8ca, 8cb, . . . , 8cm; . . . ; 8na, 8nb, . . . , 8nm which are formed at positions facing the absorptive regions 4 of the biochemical analysis unit 1 held in the cartridge 7, the mixed solution of the hybridization buffer and the probe solution flowing into the first branch passages 8a, 8b, 8c, . . . , 8n further flows into the second branch passages 8aa, 8ab, . . . , 8am; 8ba, 8bb, . . . , 8bm; 8ca, 8cb, . . . , 8cm; . . . ; 8na, 8nb, . . . , 8nm from the first branch passages 8a, 8b, 8c, . . . , 8n and is fed to the individual absorptive regions 4 of the biochemical analysis unit 1 held in the cartridge 7.

The mixed solution of the hybridization buffer and the probe solution fed to the individual absorptive regions 4 of the biochemical analysis unit 1 held in the cartridge 7 passes through the corresponding absorptive regions 4 and flows into the third branch passages 9aa, 9ab, . . . , 9am; 9ba, 9bb, . . . , 9bm; 9ca, 9cb, . . . , 9cm; . . . ; 9na, 9nb, . . . , 9nm at positions facing the second branch passages 8aa, 8ab, . . . , 8am; 8ba, 8bb, . . . , 8bm; 8ca, 8cb, . . . , 8cm; . . . ; 8na, 8nb, . . . , 8nm via the absorptive regions 4 of the biochemical analysis unit 1 held in the cartridge 7.

As a result, a substance derived from a living organism and contained in the mixed solution of the hybridization buffer and the probe solution selectively hybridizes with specific binding substances absorbed in a number of the absorptive regions 4 formed in the substrate 2 of the biochemical analysis unit 1 held in the cartridge 7.

Since the third branch passages 9aa, 9ab, . . . , 9am; 9ba, 9bb, . . . , 9bm; 9ca, 9cb, . . . , 9cm; . . . ; 9na, 9nb, . . . , 9nm merge into the fourth branch passages 9a, 9b, 9c, . . . , 9n, the mixed solution of the hybridization buffer and the probe solution flows into the fourth branch passages 9a, 9b, 9c, . . . , 9n.

Further, since the fourth branch passages 9a, 9b, 9c, . . . , 9n merge to be connected to the solution discharge passage 9 and the solution circulation pipe 14 is connected to the solution discharge passage 9, the mixed solution of the hybridization buffer and the probe solution is fed into the solution circulation pipe 14 via the fourth branch passages 9a, 9b, 9c, . .. , 9n and the solution discharge passage 9 of the cartridge 7 and recycled into the cartridge 7.

In this embodiment, since the mixed solution of the hybridization buffer and the probe solution is forcibly fed so as to pass through each of a number the absorptive regions 4 formed in the substrate 2 of the biochemical analysis unit 1 accommodated in the cartridge 7 repeatedly in this manner, it is possible to markedly increase the moving rate of a substance derived from a living organism through the absorptive regions 4 of the biochemical analysis unit 1 in comparison with the case where a substance derived from a living organism and contained in the mixed solution of the hybridization buffer and the probe solution is moved only by convection or diffusion to be hybridized with specific binding substances absorbed in a number of the absorptive regions 4 of the biochemical analysis unit 1 and, therefore, the reaction rate of hybridization can be markedly improved. Further, since it is possible to markedly improve the possibility of a substance derived from a living organism and contained in the mixed solution of the hybridization buffer and the probe solution associating with specific binding substances absorbed in deep portions of a number of the absorptive regions 4 formed in the substrate 2 of the biochemical analysis unit 1, a substance derived from a living organism and contained in mixed solution of the hybridization buffer and the probe solution can be hybridized with specific binding substances absorbed in a number of the absorptive regions 4 formed in the substrate 2 of the biochemical analysis unit 1 in a desired manner.

When a second predetermined time period has passed, the pump 15 is stopped and hybridization is completed.

The change-over valve 11b provided in the probe solution feed pipe 11a is then located at its second position where the atmosphere and the solution circulation pipe 14 communicate with each other and the change-over valve 16a provided at the bifurcated portion of the solution circulation pipe 14 and the solution discharge pipe 16 is located at its second position where the solution circulation pipe 14 and the solution discharge pipe 16 communicate with each other. The pump 15 is then driven.

As a result, the mixed solution of the hybridization buffer and the probe solution filling the inner space of the cartridge 7 and the solution circulation pipe 14 is discharged through the solution discharge pipe 16.

When the mixed solution of the hybridization buffer and the probe solution filling the inner space of the cartridge 7 and the solution circulation pipe 14 has been discharged through the solution discharge pipe 16, the change-over valve 16a provided at the bifurcated portion of the solution circulation pipe 14 and the solution discharge pipe 16 is located at its first position and the change-over valve 10b provided in the hybridization buffer feed pipe 10a, the change-over valve 11b provided in the probe solution feed pipe 11a and the change-over valve 12b provided in the antibody solution feed pipe 12a are located at their second positions where the atmosphere and the solution circulation pipe 14 communicate with each other.

The change-over valve 13b provided in the cleaning solution feed pipe 13a is then located at its first position where the cleaning solution feed pipe 13a and the solution circulation pipe 14 communicate with each other and the pump 15 is driven, thereby feeding a cleaning solution; accommodated in the cleaning solution tank 13 into the cartridge 7 via the cleaning solution feed pipe 13a and the solution circulation pipe 14.

Since the solution circulation pipe 14 is connected to the solution feed passage 8 of the cartridge 7 for a biochemical analysis unit, the cleaning solution is fed into the cartridge 7 for a biochemical analysis unit through the solution feed passage 8.

Further, since the solution feed passage 8 branches into the n first branch passages 8a, 8b, 8c, . . . , 8n , the cleaning solution flows into the n first branch passages 8a, 8b, 8c, . . . , 8n from the solution feed passage 8.

Since each of the first branch passages 8a, 8b, 8c, . . . , 8n branches into the m second branch passages 8aa, 8ab, . . . , 8am; 8ba, 8bb, . . . , 8bm; 8ca, 8cb, . . . , 8cm; . . . ; 8na, 8nb, . . . , 8nm which are formed at positions facing the absorptive regions 4 of the biochemical analysis unit 1 held in the cartridge 7, the cleaning solution flowing into the first branch passages 8a, 8b, 8c, . . . , 8n further flows into the second branch passages 8aa, 8ab, . . . , 8am; 8ba, 8bb, . . . , 8bm; 8ca, 8cb, . . . , 8cm; . . . ; 8na, 8nb, . . . , 8nm from the first branch passages 8a, 8b, 8c, . . . , 8n and is fed to the individual absorptive regions 4 of the biochemical analysis unit 1 held in the cartridge 7.

The cleaning solution fed to the individual absorptive regions 4 of the biochemical analysis unit 1 held in the cartridge 7 passes through the corresponding absorptive regions 4 and flows into the third branch passages 9aa, 9ab, . . . , 9am; 9ba, 9bb, . . . , 9bm; 9ca, 9cb, . . . , 9cm; . . . ; 9na, 9nb, . . . , 9nm at positions facing the second branch passages 8aa, 8ab, . . . , 8am; 8ba, 8bb, . . . , 8bm; 8ca, 8cb, . . . , 8cm; . . . ; 8na, 8nb, . . . , 8nm via the absorptive regions 4 of the biochemical analysis unit 1 held in the cartridge 7.

In this manner, a number of the absorptive regions 4 formed in the substrate 2 of the biochemical analysis unit 1 are cleaned with the cleaning solution.

Since the third branch passages 9aa, 9ab, . . . , 9am; 9ba, 9bb, . . . , 9bm; 9ca, 9cb, . . . , 9cm; . . . ; 9na, 9nb, . . . , 9nm merge into the fourth branch passages 9a, 9b, 9c, . . . , 9n, the cleaning solution flows into the fourth branch passages 9a, 9b, 9c, . . . , 9n.

Further, since the fourth branch passages 9a, 9b, 9c, . . . , 9n merge to be connected to the solution discharge passage 9 and the solution circulation pipe 14 is connected to the solution discharge passage 9, the cleaning solution is fed into the solution circulation pipe 14 via the fourth branch passages 9a, 9b, 9c, . . . , 9n and the solution discharge passage 9 of the cartridge 7 and recycled into the cartridge 7.

In this manner, when an inner space of the cartridge 7 and the solution circulation pipe 14 has been filled with the cleaning solution, the change-over valve 13b provided in the cleaning solution feed pipe 13a is located at its third position where communication between the cleaning solution tank 13 and the atmosphere, and the solution circulation pipe 14 is shut off and the change-over valve 10b provided in the hybridization buffer feed pipe 10a, the change-over valve 11b provided in the probe solution feed pipe 11a and the change-over valve 12b provided in the antibody solution feed pipe 12a are located at their third positions.

On the other hand, the pump 15 continues to be driven and as a result, the cleaning solution filling the inner space of the cartridge 7 and the solution circulation pipe 14 is circulated through the cartridge 7 and the solution circulation pipe 14, whereby the cleaning solution is forcibly fed so as to pass through a number of the absorptive regions 4 of the biochemical analysis unit 1 held in the cartridge 7.

In this embodiment, since the cleaning solution is forcibly fed so as to pass through a number the absorptive regions 4 formed in the substrate 2 of the biochemical analysis unit 1 held in the cartridge 7 repeatedly in this manner, even if a substance derived from a living organism which should not be hybridized with specific binding substances absorbed in the absorptive regions 4 formed in the substrate 2 of the biochemical analysis unit 1 has been bonded with the absorptive regions 4 during the process of hybridization, it is possible to very efficiently peel off and remove the substance derived from a living organism which should not be hybridized with specific binding substances absorbed in the absorptive regions 4 from a number of the absorptive regions 4 of the biochemical analysis unit 1 and the efficiency of cleaning operation can be markedly improved.

When a third predetermined time period shorter than the second predetermined time period has passed, the pump 15 is stopped and the cleaning operation is completed.

Since there is a risk of a substance derived from a living organism, which should not be hybridized with specific binding substances and has been peeled off from the absorptive regions 4 by the cleaning operation bonding with the absorptive regions 4 again, if the cleaning solution is repeatedly fed to the absorptive regions 4 of the biochemical analysis unit 1 for a long time, the pump 15 is stopped when the third predetermined time period shorter than the second predetermined time period has passed and the cleaning operation is completed.

Further, the change-over valve 13b provided in the cleaning solution feed pipe 13a is located at its second position where the atmosphere and the solution circulation pipe 14 communicate with each other and the change-over valve 16a provided at the bifurcated portion of the solution circulation pipe 14 and the solution discharge pipe 16 is located at its second position where the solution circulation pipe 14 and the solution discharge pipe 16 communicate with each other. The pump 15 is then driven.

As a result, the cleaning solution filling the inner space of the cartridge 7 and the solution circulation pipe 14 is discharged through the solution discharge pipe 16.

In this manner, radiation data of a radioactive labeling substance and a fluorescence data of a fluorescent substance such as a fluorescent dye are recorded in a number of the absorptive regions 4 formed in the substrate 2 of the biochemical analysis unit 1.

The fluorescence data recorded in a number of the absorptive regions 4 of the biochemical analysis unit 1 are read by a scanner described later and biochemical analysis data are produced.

On the other hand, radiation data recorded in a number of the absorptive regions 4 of the biochemical analysis unit 1 are transferred onto a stimulable phosphor sheet described later and read by a scanner described later, thereby producing biochemical analysis data.

To the contrary, in order to record chemiluminescence data in a number of the absorptive regions 4 formed in the substrate 2 of the biochemical analysis unit 1, an antibody solution containing an antibody to the hapten such as digoxigenin labeled with an enzyme which generates chemiluminescence emission when it contacts a chemiluminescent substrate is further prepared and accommodated in the antibody solution tank 12 and the antibody to the hapten such as digoxigenin labeled with an enzyme which generates chemiluminescence emission when it contacts a chemiluminescent substrate is bonded with the hapten such as digoxigenin labeling a substance derived from a living organism selectively hybridized with specific binding substances absorbed in a number of the absorptive regions formed in the substrate 2 of the biochemical analysis unit 1 by the an antigen-antibody reaction.

Specifically, an antibody solution containing an antibody to the hapten such as digoxigenin labeled with an enzyme which generates chemiluminescence emission when it contacts a chemiluminescent substrate is first prepared and accommodated in the antibody solution tank 12.

When the antibody solution has been accommodated in the antibody solution tank 12, the change-over valve 16a provided at the bifurcated portion of the solution circulation pipe 14 and the solution discharge pipe 16 is located at its first position and the change-over valve 10b provided in the hybridization buffer feed pipe 10a, the change-over valve 11b provided in the probe solution feed pipe 11a and the change-over valve 13b provided in the cleaning solution feed pipe 13a are located at their second positions where the atmosphere and the solution circulation pipe 14 communicate with each other.

The change-over valve 12b provided in the antibody solution feed pipe 12a is then located at its first position where the antibody solution feed pipe 12a and the solution circulation pipe 14 communicate with each other and the pump 15 is driven, thereby feeding the antibody solution accommodated in the antibody solution tank 12 into the cartridge 7 via the antibody solution feed pipe 12a and the solution circulation pipe 14.

Since the solution circulation pipe 14 is connected to the solution feed passage 8 of the cartridge 7 for a biochemical analysis unit, the antibody solution is fed into the cartridge 7 for a biochemical analysis unit through the solution feed passage 8.

Further, since the solution feed passage 8 is bifurcated to the n first branch passages 8a, 8b, 8c, . . . , 8n , the antibody solution flows into the n first branch passages 8a, 8b, 8c, . . . , 8n from the solution feed passage 8.

Since each of the first branch passages 8a, 8b, 8c, . . . , 8n branches into the m second branch passages 8aa, 8ab, . . . , 8am; 8ba, 8bb, . . . , 8bm; 8ca, 8cb, . . . , 8cm; . . . ; 8na, 8nb, . . . , 8nm which are formed at positions facing the absorptive regions 4 of the biochemical analysis unit 1 held in the cartridge 7, the antibody solution flowing into the first branch passages 8a, 8b, 8c, . . . , 8n further flows into the second branch passages 8aa, 8ab, . . . , 8am; 8ba, 8bb, . . . , 8bm; 8ca, 8cb, . . . , 8cm; . . . ; 8na, 8nb, . . . , 8nm from the first branch passages 8a, 8b, 8c, . . . , 8n and is fed to the individual absorptive regions 4 of the biochemical analysis unit 1 held in the cartridge 7.

The antibody solution fed to the individual absorptive regions 4 of the biochemical analysis unit 1 held in the cartridge 7 passes through the corresponding absorptive regions 4 and flows into the third branch passages 9aa, 9ab, . . . , 9am; 9ba, 9bb, . . . , 9bm; 9ca, 9cb, . . . , 9cm; . . . ; 9na, 9nb, . . . , 9nm at positions facing the second branch passages 8aa, 8ab, . . . , 8am; 8ba, 8bb, . . . , 8bm; 8ca, 8cb, 8cm; . . . ; 8na, 8nb, . . . , 8nm via the absorptive regions 4 of the biochemical analysis unit 1 held in the cartridge 7.

In this manner, the antibody to the hapten such as digoxigenin labeled with an enzyme which generates chemiluminescence emission when it contacts a chemiluminescent substrate is bonded with the hapten such as digoxigenin labeling a substance derived from a living organism selectively hybridized with specific binding substances absorbed in a number of the absorptive regions formed in the substrate 2 of the biochemical analysis unit 1 by the an antigen-antibody reaction.

Since the third branch passages 9aa, 9ab, . . . , 9am; 9ba, 9bb, 9bm; 9ca, 9cb, . . . , 9cm; . . . ; 9na, 9nb, . . . , 9nm merge into the fourth branch passages 9a, 9b, 9c, . . . , 9n, the antibody solution flows into the fourth branch passages 9a, 9b, 9c, . . . , 9n.

Further, since the fourth branch passages 9a, 9b, 9c, . . . , 9n merge to be connected to the solution discharge passage 9 and the solution circulation pipe 14 is connected to the solution discharge passage 9, the antibody solution is fed into the solution circulation pipe 14 via the fourth branch passages 9a, 9b, 9c, . . . , 9n and the solution discharge passage 9 of the cartridge 7 and recycled into the cartridge 7.

In this manner, when an inner space of the cartridge 7 and the solution circulation pipe 14 has been filled with the antibody solution, the change-over valve 12b provided in the antibody solution feed pipe 12a is located at its third position where communication between the antibody solution tank 12 and the atmosphere, and the solution circulation pipe 14 is shut off and the change-over valve 10b provided in the hybridization buffer feed pipe 10a, the change-over valve 11b provided in the probe solution feed pipe 11a and the change-over valve 13b provided in the cleaning solution feed pipe 13a are located at their third positions.

On the other hand, the pump 15 continues to be driven and as a result, the antibody solution filling the inner space of the cartridge 7 and the solution circulation pipe 14 is circulated through the cartridge 7 and the solution circulation pipe 14, whereby the antibody solution is forcibly fed so as to pass through a number of the absorptive regions 4 of the biochemical analysis unit 1 held in the cartridge 7.

In this embodiment, since the antibody solution is forcibly fed so as to pass through a number the absorptive regions formed in the substrate 2 of the biochemical analysis unit repeatedly in this manner, it is possible to markedly increase the moving rate of an antibody through the absorptive regions 4 of the biochemical analysis unit 1 and, therefore, the reaction rate of an antigen-antibody reaction can be markedly improved. Further, since it is possible to much more improve the possibility of an antibody for the hapten contained in the antibody solution associating with the hapten labeling a substance derived from a living organism selectively hybridized with specific binding substances absorbed in deep portions of a number of the absorptive regions 4 formed in the substrate 2 of the biochemical analysis unit 1, an antibody for the hapten contained in the antibody solution can be associated with the hapten labeling a substance derived from a living organism selectively hybridized with specific binding substances absorbed in a number of the absorptive regions 4 formed in the substrate 2 of the biochemical analysis unit 1 in a desired manner.

When a fourth predetermined time period has passed, the pump 15 is stopped and the antigen-antibody reaction is completed.

Further, the change-over valve 12b provided in the antibody solution feed pipe 12a is located at its second position where the atmosphere and the solution circulation pipe 14 communicate with each other and the change-over valve 16a provided at the bifurcated portion of the solution circulation pipe 14 and the solution discharge pipe 16 is located at its second position where the solution circulation pipe 14 and the solution discharge pipe 16 communicate with each other. The pump 15 is then driven.

As a result, the antibody solution filling the inner space of the cartridge 7 and the solution circulation pipe 14 is discharged through the solution discharge pipe 16.

When the antibody solution filling the inner space of the cartridge 7 and the solution circulation pipe 14 has been discharged through the solution discharge pipe 16, the change-over valve 16a provided at the bifurcated portion of the solution circulation pipe 14 and the solution discharge pipe 16 is located at its first position and the change-over valve 10b provided in the hybridization buffer feed pipe 10a, the change-over valve 11b provided in the probe solution feed pipe 11a and the change-over valve 12b provided in the antibody solution feed pipe 12a are located at their second positions where the atmosphere and the solution circulation pipe 14 communicate with each other.

The change-over valve 13b provided in the cleaning solution feed pipe 13a is then located at its first position where the cleaning solution feed pipe 13a and the solution circulation pipe 14 communicate with each other and the pump 15 is driven, thereby feeding a cleaning solution accommodated in the cleaning solution tank 13 into the cartridge 7 via the cleaning solution feed pipe 13a and the solution circulation pipe 14.

Since the solution circulation pipe 14 is connected to the solution feed passage 8 of the cartridge 7 for a biochemical analysis unit, the cleaning solution is fed into the cartridge 7 for a biochemical analysis unit through the solution feed passage 8.

Further, since the solution feed passage 8 branches into the n first branch passages 8a, 8b, 8c, . . . , 8n , the cleaning solution flows into the n first branch passages 8a, 8b, 8c, . . . , 8n from the solution feed passage 8.

Since each of the first branch passages 8a, 8b, 8c, . . . , 8n branches into the m second branch passages 8aa, 8ab, . . . , 8am; 8ba, 8bb, . . . , 8bm; 8ca, 8cb, . . . , 8cm; . . . ; 8na, 8nb, . . . , 8nm which are formed at positions facing the absorptive regions 4 of the biochemical analysis unit 1 held in the cartridge 7, the cleaning solution flowing into the first branch passages 8a, 8b, 8c, . . . , 8n further flows into the second branch passages 8aa, 8ab, . . . , 8am; 8ba, 8bb, . . . , 8bm; 8ca, 8cb, 8cm; . . . ; 8na, 8nb, . . . , 8nm from the first branch passages 8a, 8b, 8c, . . . , 8n and is fed to the individual absorptive regions 4 of the biochemical analysis unit 1 held in the cartridge 7.

The cleaning solution fed to the individual absorptive regions 4 of the biochemical analysis unit 1 held in the cartridge 7 passes through the corresponding absorptive regions 4 and flows into the third branch passages 9aa, 9ab, . . . , 9am; 9ba, 9bb, . . . , 9bm; 9ca, 9cb, . . . , 9cm; . . . ; 9na, 9nb, . . . , 9nm at positions facing the second branch passages 8aa, 8ab, . . . , 8am; 8ba, 8bb, . . . , 8bm; 8ca, 8cb, 8cm; . . . ; 8na, 8nb, . . . , 8nm via the absorptive regions 4 of the biochemical analysis unit 1 held in the cartridge 7.

In this manner, a number of the absorptive regions 4 formed in the substrate 2 of the biochemical analysis unit 1 are cleaned with the cleaning solution.

Since the third branch passages 9aa, 9ab, . . . , 9am; 9ba, 9bb, . . . , 9bm; 9ca, 9cb, . . . , 9cm; . . . ; 9na, 9nb, . . . , 9nm merge into the fourth branch passages 9a, 9b, 9c, . . . , 9n, the cleaning solution flows into the fourth branch passages 9a, 9b, 9c, . . . , 9n.

Further, since the fourth branch passages 9a, 9b, 9c, . . . , 9n merge to be connected to the solution discharge passage 9 and the solution circulation pipe 14 is connected to the solution discharge passage 9, the cleaning solution is fed into the solution circulation pipe 14 via the fourth branch passages 9a, 9b, 9c, . . . , 9n and the solution discharge passage 9 of the cartridge 7 and recycled into the cartridge 7.

In this manner, when an inner space of the cartridge 7 and the solution circulation pipe 14 has been filled with the cleaning solution, the change-over valve 13b provided in the cleaning solution feed pipe 13a is located at its third position where communication between the cleaning solution tank 13 and the atmosphere, and the solution circulation pipe 14 is shut off and the change-over valve 10b provided in the hybridization buffer feed pipe 10a, the change-over valve 11b provided in the probe solution feed pipe 11a and the change-over valve 12b provided in the antibody solution feed pipe 12a are located at their third positions.

On the other hand, the pump 15 continues to be driven and as a result, the cleaning solution filling the inner space of the cartridge 7 and the solution circulation pipe 14 is circulated through the cartridge 7 and the solution circulation pipe 14, whereby the cleaning solution is forcibly fed so as to pass through a number of the absorptive regions 4 of the biochemical analysis unit 1 held in the cartridge 7.

In this embodiment, since the cleaning solution is forcibly fed so as to pass through a number the absorptive regions 4 formed in the substrate 2 of the biochemical analysis unit 1 accommodated in the cartridge 7 repeatedly in this manner, even if an antibody which should not be bonded with the hapten labeling a substance derived from a living body and selectively hybridized with specific binding substances absorbed in the absorptive regions 4 of the biochemical analysis unit 1 has been bonded with the absorptive regions 4 formed in the substrate 2 of the biochemical analysis unit 1, it is possible to very efficiently peel off and remove the antibody which should not be bonded with the hapten from a number of the absorptive regions 4 of the biochemical analysis unit 1 and the efficiency of cleaning operation can be markedly improved.

When a fifth predetermined time period shorter than the fourth predetermined time period has passed, the pump 15 is stopped and the cleaning operation is completed.

Further, the change-over valve 13b provided in the cleaning solution feed pipe 13a is located at its second position where the atmosphere and the solution circulation pipe 14 communicate with each other and the change-over valve 16a provided at the bifurcated portion of the solution circulation pipe 14 and the solution discharge pipe 16 is located at its second position where the solution circulation pipe 14 and the solution discharge pipe 16 communicate with each other. The pump 15 is then driven.

As a result, the cleaning solution filling the inner space of the cartridge 7 and the solution circulation pipe 14 is discharged through the solution discharge pipe 16.

As described above, chemiluminescence data are recorded in a number of the absorptive regions 4 formed in the substrate 2 of the biochemical analysis unit 1.

Chemiluminescence data recorded in a number of the absorptive regions 4 of the biochemical analysis unit 1 are read by a cooled CCD camera of a data producing system described later or transferred onto a stimulable phosphor sheet described later and read by a scanner described later, thereby producing biochemical analysis data.

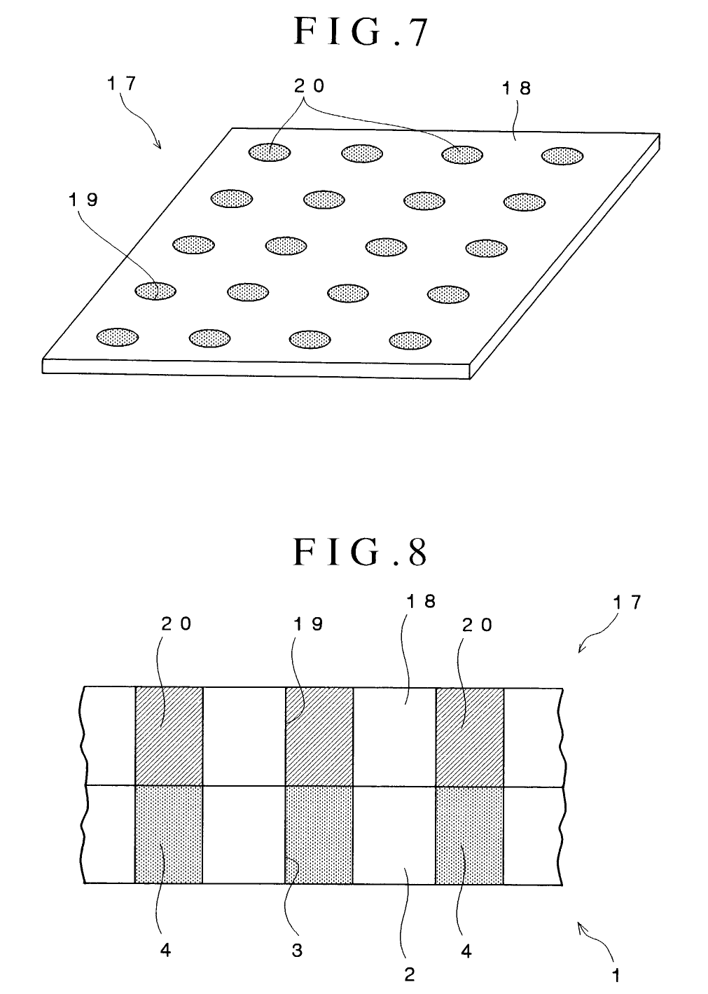

FIG. 7 is a schematic perspective view showing a stimulable phosphor sheet onto which radiation data are to be transferred.

As shown in FIG. 7, a stimulable phosphor sheet 17 according to this embodiment includes a support 18 made of stainless steel and regularly formed with a number of substantially circular through-holes 19 and a number of stimulable phosphor layer regions 20 are dot-like formed by charging BaFX system stimulable phosphor (where X is at least one halogen atom selected from the group consisting of Cl, Br and I) capable of absorbing and storing radiation energy in the through-holes 19.

A number of the through-holes 19 are formed in the support 18 in the same pattern as that of a number of the absorptive regions 4 formed in the substrate 2 of the biochemical analysis unit 1 and each of them has the same size as that of the absorptive region 4 formed in the substrate 2 of the biochemical analysis unit 1.

Therefore, although not accurately shown in FIG. 7, in this embodiment, about 10,000 substantially circular stimulable phosphor layer regions 20 having a size of about 0.01 mm² are dot-like formed in a regular pattern at a density of about 5,000 per cm² in the support 18 of the stimulable phosphor sheet 17.

In this embodiment, the stimulable phosphor sheet 17 is formed by charging stimulable phosphor in a number of the through-holes 19 formed in the support 18 in such a manner that the surfaces of the stimulable phosphor layer regions 20 lie at the same height level of that of the surface of the support 18.

FIG. 8 is a schematic cross-sectional view showing a method for exposing a number of the stimulable phosphor layer regions 20 formed in the stimulable phosphor sheet 17 by a radioactive labeling substance contained in a number of the absorptive regions 4 formed in the biochemical analysis unit 1.

As shown in FIG. 7, when the stimulable phosphor layer regions 20 of a stimulable phosphor sheet 17 are to be exposed, the stimulable phosphor sheet 17 is superposed on the biochemical analysis unit 1 in such a manner that each of the stimulable phosphor layer regions 20 formed in the support 18 of the stimulable phosphor sheet 17 faces the corresponding absorptive regions 4 formed in the substrate 2 of the biochemical analysis unit 1.

In this embodiment, since the biochemical analysis unit 1 is formed by charging nylon-6 in a number of the through-holes 3 formed in the substrate 2 made of stainless steel, the biochemical analysis unit 1 does not stretch or shrink even when it is subjected to liquid processing such as hybridization and, therefore, it is possible to easily and accurately superpose the stimulable phosphor sheet 17 on the biochemical analysis unit 1 so that each of the stimulable phosphor layer regions 20 formed in the support 18 of the stimulable phosphor sheet 17 accurately faces the corresponding absorptive region 4 formed in the substrate 2 of the biochemical analysis unit 1, thereby exposing the stimulable phosphor layer regions 20.

In this manner, each of the stimulable phosphor layer regions 20 formed in the support 18 of the stimulable phosphor sheet 17 is kept to face the corresponding absorptive region 4 formed in the substrate 2 of the biochemical analysis unit 1 for a predetermined time period, whereby a number of the stimulable phosphor layer regions 20 formed in the support 18 of the stimulable phosphor sheet 17 are exposed to the radioactive labeling substance contained in a number of the absorptive regions 4 formed in the substrate 2 of the biochemical analysis unit 1.

During the exposure operation, electron beams (β rays) are released from the radioactive labeling substance contained in the absorptive regions 4 of the biochemical analysis unit 1. However, since a number of the absorptive regions 4 of the biochemical analysis unit 1 are formed spaced apart from each other in the substrate 2 made of stainless steel and the substrate 2 made of stainless steel capable of attenuating radiation energy is present around each of the absorptive regions 4, electron beams (β rays) released from the radioactive labeling substance contained in the absorptive regions 4 of the biochemical analysis unit 1 can be efficiently prevented from scattering in the substrate 2 of the biochemical analysis unit 1. Further, since a number of the stimulable phosphor layer regions 20 of the stimulable phosphor sheet 17 are formed by charging stimulable phosphor in a number of the through-holes 19 formed in the support 18 made of stainless steel capable of attenuating radiation energy and the support 18 made of stainless steel is present around each of the stimulable phosphor layer regions 20, electron beams (β rays) released from the radioactive labeling substance contained in the absorptive regions 4 of the biochemical analysis unit 1 can be efficiently prevented from scattering in the support 18 of the stimulable phosphor sheet 17. Therefore, it is possible to cause all electron beams (β rays) released from the radioactive labeling substance contained in the absorptive region 4 to enter the stimulable phosphor layer region 20 the absorptive region 4 faces and to effectively prevent electron beams (β rays) released from the absorptive region 4 from entering stimulable phosphor layer regions 20 to be exposed to electron beams (β rays) released from neighboring absorptive regions 4.

In this manner, a number of the stimulable phosphor layer regions 20 formed in the support 18 of the stimulable phosphor sheet 17 can be selectively exposed to a radioactive labeling substance contained in the corresponding absorptive region 4 of the biochemical analysis unit 1.

Thus, radiation data of a radioactive labeling substance are recorded in a number of the stimulable phosphor layer regions 20 formed in the support 18 of the stimulable phosphor sheet 17.

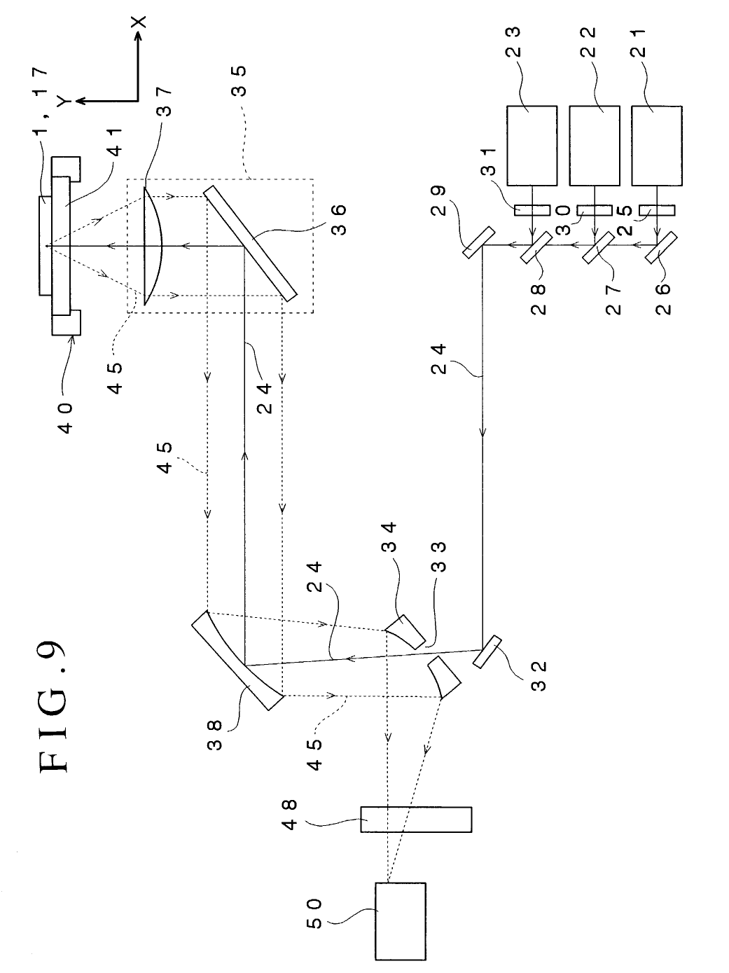

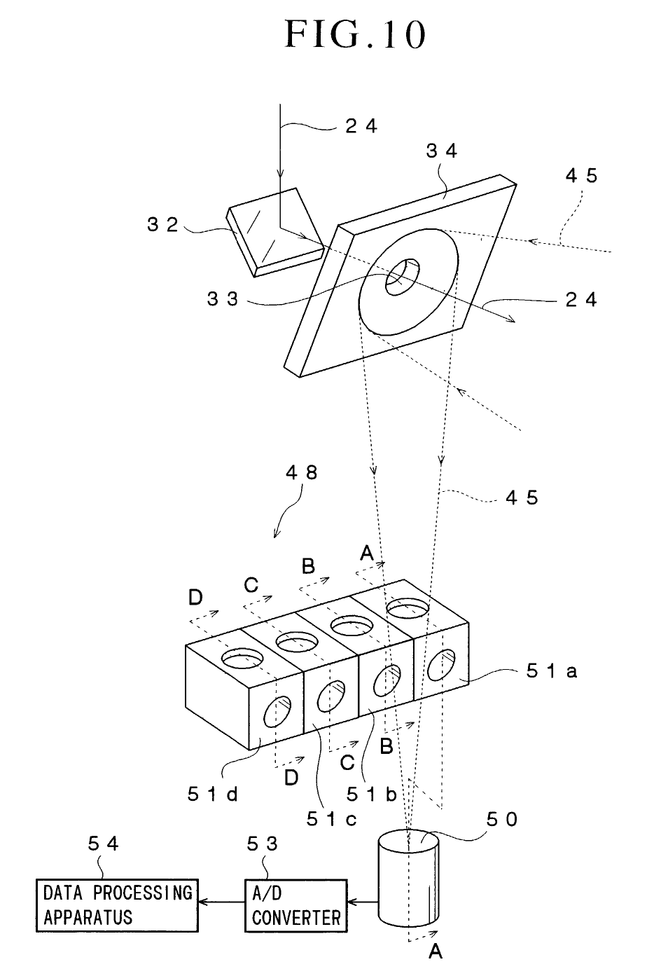

FIG. 9 is a schematic view showing a scanner for reading radiation data recorded in a number of the stimulable phosphor layer regions 20 formed in the support 18 of the stimulable phosphor sheet 17 and fluorescence data of a fluorescent substance such as a fluorescent dye recorded in a number of the absorptive regions 4 formed in the substrate 2 of the biochemical analysis unit 1 to produce biochemical analysis data and FIG. 10 is a schematic perspective view showing details in the vicinity of a photomultiplier of a scanner shown in FIG. 9.

As shown in FIG. 9, the scanner includes a first laser stimulating ray source 21 for emitting a laser beam having a wavelength of 640 nm, a second laser stimulating ray source 22 for emitting a laser beam having a wavelength of 532 nm and a third laser stimulating ray source 23 for emitting a laser beam having a wavelength of 473 nm.

In this embodiment, the first laser stimulating ray source 21 is constituted by a semiconductor laser beam source and the second laser stimulating ray source 22 and the third laser stimulating ray source 23 are constituted by a second harmonic generation element.

A laser beam 24 emitted from the first laser stimulating source 21 passes through a collimator lens 25, thereby being made a parallel beam, and is reflected by a mirror 26. A first dichroic mirror 27 for transmitting light having a wavelength of 640 nm but reflecting light having a wavelength of 532 nm and a second dichroic mirror 28 for transmitting light having a wavelength equal to and longer than 532 nm but reflecting light having a wavelength of 473 nm are provided in the optical path of the laser beam 24 emitted from the first laser stimulating ray source 21. The laser beam 24 emitted from the first laser stimulating ray source 21 and reflected by the mirror 26 passes through the first dichroic mirror 27 and the second dichroic mirror 28 and advances to a mirror 29.

On the other hand, the laser beam 24 emitted from the second laser stimulating ray source 22 passes through a collimator lens 30, thereby being made a parallel beam, and is reflected by the first dichroic mirror 27, thereby changing its direction by 90 degrees. The laser beam 24 then passes through the second dichroic mirror 28 and advances to the mirror 29.

Further, the laser beam 24 emitted from the third laser stimulating ray source 23 passes through a collimator lens 31, thereby being made a parallel beam, and is reflected by the second dichroic mirror 28, thereby changing its direction by 90 degrees. The laser beam 24 then advances to the mirror 29.

The laser beam 24 advancing to the mirror 29 is reflected by the mirror 29 and advances to a mirror 32 to be reflected thereby.

A perforated mirror 34 formed with a hole 33 at the center portion thereof is provided in the optical path of the laser beam 24 reflected by the mirror 32. The laser beam 24 reflected by the mirror 32 passes through the hole 33 of the perforated mirror 34 and advances to a concave mirror 38.

The laser beam 24 advancing to the concave mirror 38 is reflected by the concave mirror 38 and enters an optical head 35.

The optical head 35 includes a mirror 36 and an aspherical lens 37 and the laser beam 24 entering the optical head 35 is reflected by the mirror 36 and condensed by the aspherical lens 37 onto the stimulable phosphor sheet 17 or the biochemical analysis unit 1 placed on the glass plate 41 of a stage 40.

When the laser beam 24 impinges on one of the stimulable phosphor layer regions 17 formed in the support 16 of the stimulable phosphor sheet 15, stimulable phosphor contained in the stimulable phosphor layer region 17 is excited, thereby releasing stimulated emission 45. On the other hand, when the laser beam 24 impinges on one of the absorptive regions 4 formed in the substrate 2 of the biochemical analysis unit 1, a fluorescent dye or the like contained in the absorptive region 4 is excited, thereby releasing fluorescence emission 45.

The stimulated emission 45 released from the stimulable phosphor layer region 20 formed in the support 18 of the stimulable phosphor 17 or the fluorescence emission 45 released from the absorptive region 4 formed in the substrate 2 of the biochemical analysis unit 1 is condensed onto the mirror 36 by the aspherical lens 37 provided in the optical head 35 and reflected by the mirror 36 on the side of the optical path of the laser beam 24, thereby being made a parallel beam to advance to the concave mirror 38.

The stimulated emission 45 or the fluorescence emission 45 advancing to the concave mirror 38 is reflected by the concave mirror 38 and advances to the perforated mirror 34.

As shown in FIG. 10, the stimulated emission 45 or the fluorescence emission 45 advancing to the perforated mirror 34 is reflected downward by the perforated mirror 34 formed as a concave mirror and advances to a filter unit 48, whereby light having a predetermined wavelength is cut. The stimulated emission 45 or the fluorescence emission 45 then impinges on a photomultiplier 50, thereby being photoelectrically detected.

As shown in FIG. 10, the filter unit 48 is provided with four filter members 51a, 51b, 51c and 51d and is constituted to be laterally movable in FIG. 10 by a motor (not shown).

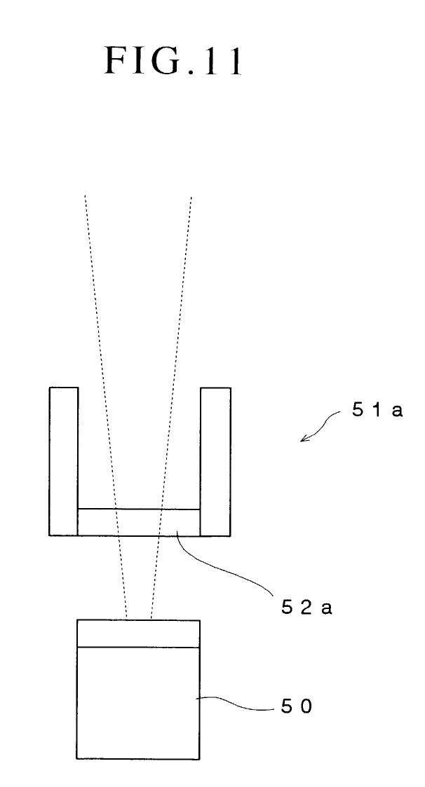

FIG. 11 is a schematic cross-sectional view taken along a line A-A in FIG. 10.

As shown in FIG. 11, the filter member 51a includes a filter 52a and the filter 52a is used for reading fluorescence emission 45 by stimulating a fluorescent substance such as a fluorescent dye contained in a number of the absorptive regions 4 formed in the substrate 2 of the biochemical analysis unit 1 using the first laser stimulating ray source 21 and has a property of cutting off light having a wavelength of 640 nm but transmitting light having a wavelength longer than 640 nm.

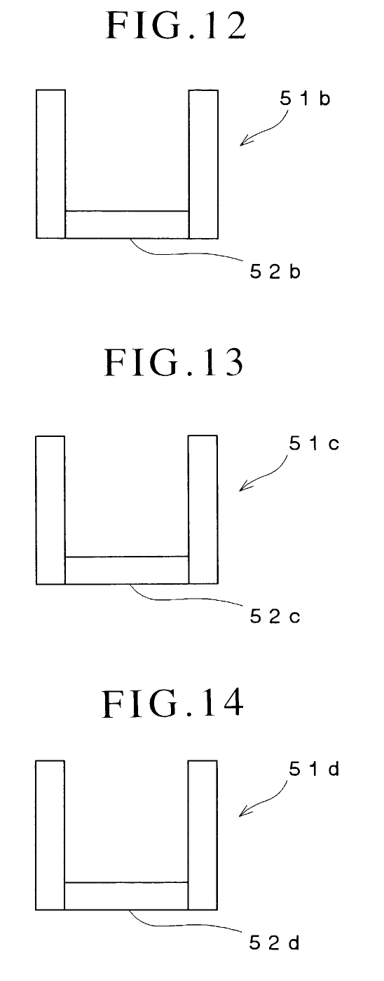

FIG. 12 is a schematic cross-sectional view taken along a line B-B in FIG. 10.

As shown in FIG. 12, the filter member 51b includes a filter 52b and the filter 52b is used for reading fluorescence emission 45 by stimulating a fluorescent substance such as a fluorescent dye contained in a number of the absorptive regions 4 formed in the substrate 2 of the biochemical analysis unit 1 using the second laser stimulating ray source 22 and has a property of cutting off light having a wavelength of 532 nm but transmitting light having a wavelength longer than 532 nm.

FIG. 13 is a schematic cross-sectional view taken along a line C-C in FIG. 10.

As shown in FIG. 13, the filter member 51c includes a filter 52c and the filter 52c is used for reading fluorescence emission 45 by stimulating a fluorescent substance such as a fluorescent dye contained in in a number of the absorptive regions 4 formed in the substrate 2 of the biochemical analysis unit 1 using the third laser stimulating ray source 23 and has a property of cutting off light having a wavelength of 473 nm but transmitting light having a wavelength longer than 473 nm.

FIG. 14 is a schematic cross-sectional view taken along a line D-D in FIG. 10.

As shown in FIG. 14, the filter member 51d includes a filter 52d and the filter 52d is used for reading stimulated emission 45 released from stimulable phosphor contained in the stimulable phosphor layer 20 formed in the support 18 of the stimulable phosphor sheet 17 upon being stimulated using the first laser stimulating ray source 1 and has a property of transmitting only light having a wavelength corresponding to that of stimulated emission 45 emitted from stimulable phosphor and cutting off light having a wavelength of 640 nm.

Therefore, in accordance with the kind of a stimulating ray source to be used, one of these filter members 51a, 51b, 51c, 51d is selectively positioned in front of the photomultiplier 50, thereby enabling the photomultiplier 50 to photoelectrically detect only light to be detected.

The analog data produced by photoelectrically detecting stimulated emission 45 or fluorescence emission 45 with the photomultiplier 50 are converted by an A/D converter 53 into digital data and the digital data are fed to a data processing apparatus 54.

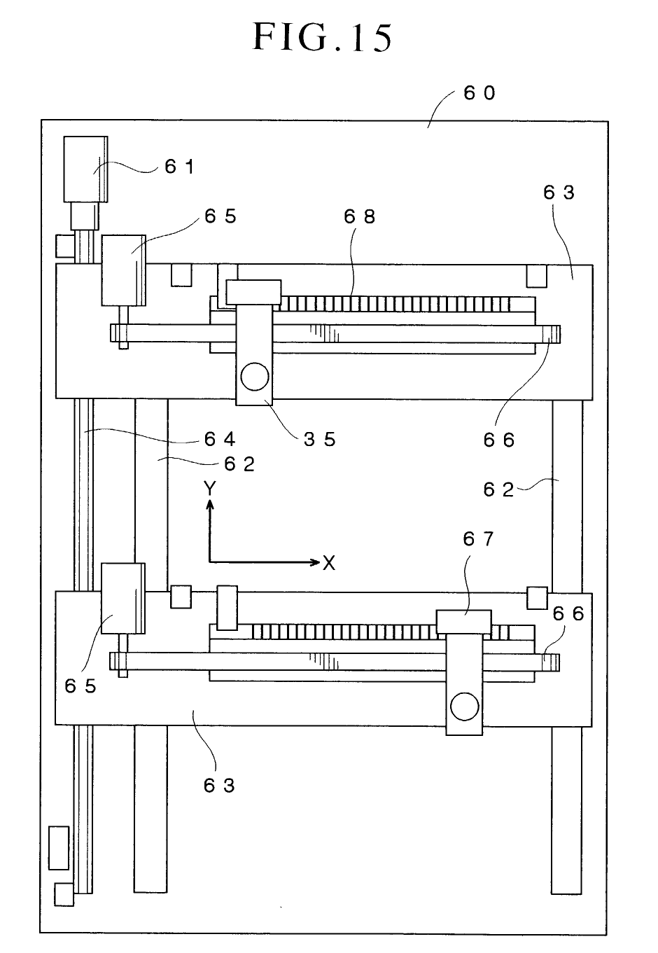

FIG. 15 is a schematic plan view showing the scanning mechanism of the optical head 35.

In FIG. 15, optical systems other than the optical head 35 and the paths of the laser beam 24 and stimulated emission 45 or fluorescence emission 45 are omitted for simplification.

As shown in FIG. 15, the scanning mechanism of the optical head 35 includes a base plate 60, and a sub-scanning pulse motor 61 and a pair of rails 62, 62 are fixed on the base plate 60. A movable base plate 63 is further provided so as to be movable in the sub-scanning direction indicated by an arrow Y in FIG. 15.

The movable base plate 63 is formed with a threaded hole (not shown) and a threaded rod 64 rotated by the sub-scanning pulse motor 61 is engaged with the inside of the hole.

A main scanning stepping motor 65 is provided on the movable base plate 63. The main scanning stepping motor 65 is adapted for intermittently driving an endless belt 66 at a pitch equal to the distance between neighboring absorptive regions 4 formed in the substrate 2 of the biochemical analysis unit 1, namely, the distance between neighboring stimulable phosphor layer regions 20 formed in the support 18 of the stimulable phosphor sheet 17.

The optical head 35 is fixed to the endless belt 66 and when the endless belt 66 is driven by the main scanning stepping motor 65, the optical head 35 is moved in the main scanning direction indicated by an arrow X in FIG. 15.

In FIG. 15, the reference numeral 67 designates a linear encoder for detecting the position of the optical head 35 in the main scanning direction and the reference numeral 68 designates slits of the linear encoder 67.

Therefore, the optical head 35 is moved in the main scanning direction indicated by the arrow X and the sub-scanning direction indicated by the arrow Y in FIG. 15 by driving the endless belt 66 in the main scanning direction by the main scanning stepping motor 65 and intermittently moving the movable base plate 63 in the sub-scanning direction by the sub-scanning pulse motor 61, thereby scanning all of the stimulable phosphor layer regions 20 formed in the support 18 of the stimulable phosphor sheet 17 or all of the absorptive regions 4 formed in the substrate 2 of the biochemical analysis unit 1 with the laser beam 24.

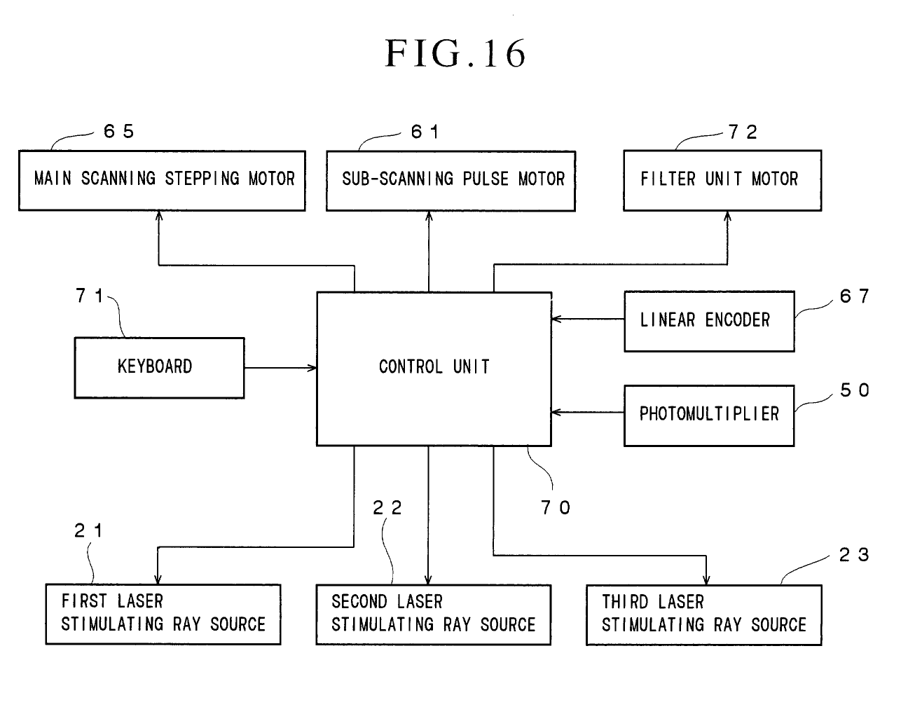

FIG. 16 is a block diagram of a control system, an input system, a drive system and a detection system of the scanner shown in FIG. 9.

As shown in FIG. 16, the control system of the scanner includes a control unit 70 for controlling the overall operation of the scanner and the input system of the scanner includes a keyboard 71 which can be operated by a user and through which various instruction signals can be input.

As shown in FIG. 16, the drive system of the scanner includes the main scanning stepping motor 65 for intermittently moving the optical head 35 in the main scanning direction, the sub-scanning pulse motor 61 for moving the optical head 35 in the sub-scanning direction and a filter unit motor 72 for moving the filter unit 48 provided with the four filter members 51a, 51b, 51c and 51d.

The control unit 70 is adapted for selectively outputting a drive signal to the first laser stimulating ray source 21, the second laser stimulating ray source 22 or the third laser stimulating ray source 23 and outputting a drive signal to the filter unit motor 72.

As shown in FIG. 16, the detection system of the scanner includes the photomultiplier 50 and the linear encoder 67 for detecting the position of the optical head 35 in the main scanning direction.

In this embodiment, the control unit 70 is adapted to control the on and off operation of the first laser stimulating ray source 21, the second laser stimulating ray source 22 or the third laser stimulating ray source 23 in accordance with a detection signal indicating the position of the optical head 35 input from the linear encoder 67.

The thus constituted scanner reads radiation data recorded in a number of the stimulable phosphor layer regions 20 formed in the support 18 of the stimulable phosphor sheet 17 and produces biochemical analysis data in the following manner.

A stimulable phosphor sheet 17 is first set on the glass plate 41 of the stage 40 by a user.

An instruction signal indicating that radiation data recorded in the stimulable phosphor layer region 17 formed in the support 16 of the stimulable phosphor sheet 15 are to be read is then input through the keyboard 71.

The instruction signal input through the keyboard 71 is input to the control unit 70 and the control unit 70 outputs a drive signal to the filter unit motor 72 in accordance with the instruction signal, thereby moving the filter unit 48 so as to locate the filter member 51d provided with the filter 52d having a property of transmitting only light having a wavelength corresponding to that of stimulated emission emitted from stimulable phosphor but cutting off light having a wavelength of 640 nm in the optical path of stimulated emission 45.

The control unit 70 further outputs a drive signal to the main scanning stepping motor 65 to move the optical head 35 in the main scanning direction and when it determines based on a detection signal indicating the position of the optical head 35 input from the linear encoder 67 that the optical head 35 has reached a position where a laser beam 24 can be projected onto a first stimulable phosphor layer region 20 among a number of the stimulable phosphor layer regions 20 formed in the support 18 of the stimulable phosphor sheet 17, it outputs a drive stop signal to the main scanning stepping motor 65 and a drive signal to the first stimulating ray source 21, thereby actuating it to emit a laser beam 24 having a wavelength of 640 nm.

A laser beam 24 emitted from the first laser stimulating source 21 passes through the collimator lens 25, thereby being made a parallel beam, and is reflected by the mirror 26.

The laser beam 24 reflected by the mirror 26 passes through the first dichroic mirror 27 and the second dichroic mirror 28 and advances to the mirror 29.

The laser beam 24 advancing to the mirror 29 is reflected by the mirror 29 and advances to the mirror 32 to be reflected thereby.

The laser beam 24 reflected by the mirror 32 passes through the hole 33 of the perforated mirror 34 and advances to the concave mirror 38.

The laser beam 24 advancing to the concave mirror 38 is reflected by the concave mirror 38 and enters the optical head 35.

The laser beam 24 entering the optical head 35 is reflected by the mirror 36 and condensed by the aspherical lens 37 onto the first stimulable phosphor layer region 20 of the stimulable phosphor sheet 17 placed on the glass plate 41 of a stage 40.

In this embodiment, since the stimulable phosphor layer regions 20 are formed by charging stimulable phosphor in a number of the through-holes 19 formed in the support 18 made of stainless steel capable of attenuating light energy, it is possible to effectively prevent the laser beam 24 from scattering in each of the stimulable phosphor layer regions 20 and entering the neighboring stimulable phosphor layer regions 20 to excite stimulable phosphor contained in the neighboring stimulable phosphor layer regions 20.

When the laser beam 24 impinges onto the first stimulable phosphor layer region 20 formed in the support 18 of the stimulable phosphor sheet 17, stimulable phosphor contained in the first stimulable phosphor layer region 20 is excited by the laser beam 24, thereby releasing stimulated emission 45 from the first stimulable phosphor layer region 20.

The stimulated emission 45 released from the first stimulable phosphor layer region 20 is condensed onto the mirror 36 by the aspherical lens 37 provided in the optical head 35 and reflected by the mirror 36 on the side of the optical path of the laser beam 24, thereby being made a parallel beam to advance to the concave mirror 38.

The stimulated emission 45 advancing to the concave mirror 38 is reflected by the concave mirror 38 and advances to the perforated mirror 34.

As shown in FIG. 10, the stimulated emission 45 advancing to the perforated mirror 34 is reflected downward by the perforated mirror 34 formed as a concave mirror and advances to the filter 52d of the filter unit 48.

Since the filter 52d has a property of transmitting only light having a wavelength corresponding to that of stimulated emission emitted from stimulable phosphor and cutting off light having a wavelength of 640 nm, light having a wavelength of 640 nm corresponding to that of the stimulating ray is cut off by the filter 52d and only light having a wavelength corresponding to that of stimulated emission released from the first stimulable phosphor layer region 20 passes through the filter 52d to be photoelectrically detected by the photomultiplier 50.

Analog data produced by photoelectrically detecting stimulated emission 45 with the photomultiplier 50 are converted by the A/D converter 53 into digital data and the digital data are fed to the data processing apparatus 54.

When a predetermined time, for example, several microseconds, has passed after the first stimulating ray source 21 was turned on, the control unit 70 outputs a drive stop signal to the first stimulating ray source 21, thereby turning it off and outputs a drive signal to the main scanning stepping motor 65, thereby moving the optical head 35 by one pitch equal to the distance between neighboring stimulable phosphor layer regions 20 formed in the stimulable phosphor sheet 17.

When the control unit 70 determines based on a detection signal indicating the position of the optical head 35 input from the linear encoder 67 that the optical head 35 has been moved by one pitch equal to the distance between neighboring stimulable phosphor layer regions 20 and has reached a position where a laser beam 24 can be projected onto a second stimulable phosphor layer region 20 next to the first stimulable phosphor layer region 20 formed in the support 18 of the stimulable phosphor sheet 17, it outputs a drive signal to the first stimulating ray source 21 to turn it on, thereby causing the laser beam 24 to excite stimulable phosphor contained in the second stimulable phosphor layer region 20 formed in the support 18 of the stimulable phosphor sheet 17 next to the first stimulable phosphor layer region 20.

Similarly to the above, the second stimulable phosphor layer region 20 formed in the support 18 of the stimulable phosphor sheet 17 is irradiated with the laser beam 24 emitted from the first laser stimulating ray source 21 for a predetermined time and when biochemical analysis data have been produced from radiation data recorded in the second stimulable phosphor layer region 20 by photoelectrically detecting stimulated emission 45 released from the second stimulable phosphor layer region 20 in response to the excitation of stimulable phosphor with the photomultiplier 50 to produce analog data and digitizing the analog data by the A/D converter 53, the control unit 70 outputs a drive stop signal to the first stimulating ray source 21, thereby turning it off and outputs a drive signal to the main scanning stepping motor 65, thereby moving the optical head 35 by one pitch equal to the distance between neighboring stimulable phosphor layer regions 20.

In this manner, the on and off operation of the first stimulating ray source 21 is repeated in synchronism with the intermittent movement of the optical head 35 and when the control unit 70 determines based on a detection signal indicating the position of the optical head 35 input from the linear encoder 67 that the optical head 35 has been moved by one scanning line in the main scanning direction and that the stimulable phosphor layer regions 20 included in a first line of the stimulable phosphor layer regions 20 formed in the support 18 of the stimulable phosphor sheet 17 have been scanned with the laser beam 24, it outputs a drive signal to the main scanning stepping motor 65, thereby returning the optical head 35 to its original position and outputs a drive signal to the sub-scanning pulse motor 61, thereby causing it to move the movable base plate 63 by one scanning line in the sub-scanning direction.

When the control unit 70 determines based on a detection signal indicating the position of the optical head 35 input from the linear encoder 67 that the optical head 35 has been returned to its original position and determines that the movable base plate 63 has been moved by one scanning line in the sub-scanning direction, similarly to the manner in which the stimulable phosphor layer regions 20 included in the first line of the stimulable phosphor layer regions 20 formed in the support 18 of the stimulable phosphor sheet 17 were sequentially irradiated with the laser beam 24 emitted from the first laser stimulating ray source 21, the stimulable phosphor layer regions 20 included in a second line of the stimulable phosphor layer regions 20 formed in the support 18 of the stimulable phosphor sheet 17 are sequentially irradiated with the laser beam 24 emitted from the first laser stimulating ray source 21, thereby exciting stimulable phosphor contained in the stimulable phosphor layer regions 20 included in the second line and stimulated emission 45 released from the stimulable phosphor layer regions 20 in the second line is sequentially and photoelectrically detected by the photomultiplier 50.

Analog data produced by photoelectrically detecting stimulated emission 45 with the photomultiplier 50 are converted by an A/D converter 53 into digital data, thereby producing biochemical analysis data from radiation data recorded in the stimulable phosphor layer regions 20 formed in the support 18 of the stimulable phosphor sheet 17.

When all of the stimulable phosphor layer regions 20 formed in the support 18 of the stimulable phosphor sheet 17 have been scanned with the laser beam 24 emitted from the first laser stimulating ray source 21 to excite stimulable phosphor contained in the stimulable phosphor layer regions 20 and biochemical analysis data produced from radiation data recorded in the stimulable phosphor layer regions 20 formed in the support 18 of the stimulable phosphor sheet 17 by photoelectrically detecting stimulated emission 45 released from the stimulable phosphor layer regions 20 with the photomultiplier 50 to produce analog data and digitizing the analog data by the AID converter 53 have been forwarded to the data processing apparatus 54, the control unit 70 outputs a drive stop signal to the first laser stimulating ray source 21, thereby turning it off.

As described above, radiation data of the radioactive labeling substance recorded in a number of the stimulable phosphor layer regions 17 of the stimulable phosphor sheet 15 are read by the first scanner to produce biochemical analysis data.

On the other hand, when fluorescence data of a fluorescent substance recorded in a number of the absorptive regions 4 formed in the substrate 2 of the biochemical analysis unit 1 are to be read to produce biochemical analysis data, the biochemical analysis unit 1 is first set by the user on the glass plate 41 of the stage 40.

An instruction signal indicating that fluorescence data recorded in a number of the absorptive regions 4 formed in the substrate 2 of the biochemical analysis unit 1 are to be read is then input by the user through the keyboard 71 together with a labeling substance identifying signal for identifying the kind of a fluorescent substance such as a fluorescent dye labeling a substance derived from a living organism.

When the instruction signal and the labeling substance identifying signal are input by the user through the keyboard 71, the control unit 70 selects based on the instruction signal and the labeling substance identifying signal a laser stimulating ray source for emitting a laser beam 24 of a wavelength capable of efficiently stimulating the input fluorescent substance from among the first laser stimulating ray source 21, the second laser stimulating ray source 22 and the third laser stimulating ray source 23 and selects the filter member for cutting light having a wavelength of the laser beam 24 to be used for stimulating the input fluorescent substance and transmitting light having a longer wavelength than that of the laser beam to be used for stimulation from among the three filter members 51a, 51b and 51c.

For example, when Rhodamine (registered trademark), which can be most efficiently stimulated by a laser beam having a wavelength of 532 nm, is used as a fluorescent substance for labeling a substance derived from a living organism and a signal indicating such a fact is input, the control unit 70 selects the second laser stimulating ray source 22 and the filter 52b and outputs a drive signal to the filter unit motor 72, thereby moving the filter unit 48 so that the filter member 51b inserting the filter 52b having a property of cutting off light having a wavelength of 532 nm but transmitting light having a wavelength longer than 532 nm in the optical path of the fluorescence emission 45 to be released from the biochemical analysis unit 1.

The control unit 70 further outputs a drive signal to the main scanning stepping motor 65 to move the optical head 35 in the main scanning direction and when it determines based on a detection signal indicating the position of the optical head 35 input from the linear encoder 67 that the optical head 35 has reached a position where a laser beam 24 can be projected onto a first absorptive region 4 among a number of the absorptive regions 4 formed in the substrate 2 of the biochemical analysis unit 1, it outputs a drive stop signal to the main scanning stepping motor 65 and a drive signal to the second laser stimulating ray source 22, thereby actuating it to emit a laser beam 24 having a wavelength of 532 nm.

The laser beam 24 emitted from the second laser stimulating ray source 22 is made a parallel beam by the collimator lens 30, advances to the first dichroic mirror 27 and is reflected thereby.

The laser beam 24 reflected by the first dichroic mirror 27 transmits through the second dichroic mirror 28 and advances to the mirror 29.

The laser beam 24 advancing to the mirror 29 is reflected by the mirror 29 and further advances to the mirror 32 to be reflected thereby.

The laser beam 24 reflected by the mirror 32 advances to the perforated mirror 34 and passes through the hole 33 of the perforated mirror 34. Then, the laser beam 24 advances to the concave mirror 38.

The laser beam 24 advancing to the concave mirror 38 is reflected thereby and enters the optical head 35.

The laser beam 24 entering the optical head 35 is reflected by the mirror 36 and condensed by the aspherical lens 37 onto the first absorptive region 4 of the biochemical analysis unit 1 placed on the glass plate 41 of the stage 40.

In this embodiment, since each of the absorptive regions 4 of the biochemical analysis unit 1 is formed by charging nylon-6 in the through-hole 3 formed in the substrate 2 made of stainless steel and the substrate 2 capable of attenuating light energy are present around each of the absorptive regions 4 of the biochemical analysis unit 1, it is possible to effectively prevent the laser beam 24 from scattering in each of the absorptive regions 4 and entering the neighboring absorptive regions 4 to excite a fluorescent substance contained in the neighboring absorptive regions 4.

When the laser beam 24 impinges onto the first absorptive region 4 formed in the biochemical analysis unit 1, a fluorescent substance such as a fluorescent dye, for instance, Rhodamine, contained in the absorptive region 4 formed in the biochemical analysis unit 1 is stimulated by the laser beam 24 and fluorescence emission 45 is released from Rhodamine.

In this embodiment, since each of the absorptive regions 4 of the biochemical analysis unit 1 is formed by charging nylon-6 in the through-hole 3 formed in the substrate 2 made of stainless steel and the substrate 2 capable of attenuating light energy are present around each of the absorptive regions 4 of the biochemical analysis unit 1, it is possible to effectively prevent fluorescence emission 45 released from a fluorescent substance from scattering in the biochemical analysis unit 1 and being mixed with fluorescence emission 45 released from a fluorescent substance contained in the neighboring absorptive regions 4.

The fluorescence emission 45 released from Rhodamine is condensed by the aspherical lens 37 provided in the optical head 35 and reflected by the mirror 36 on the side of an optical path of the laser beam 24, thereby being made a parallel beam to advance to the concave mirror 38.

The fluorescence emission 45 advancing to the concave mirror 38 is reflected by the concave mirror 38 and advances to the perforated mirror 34.

As shown in FIG. 10, the fluorescence emission 45 advancing to the perforated mirror 34 is reflected downward by the perforated mirror 34 formed as a concave mirror and advances to the filter 52b of a filter unit 48.

Since the filter 52b has a property of cutting off light having a wavelength of 532 nm but transmitting light having a wavelength longer than 532 nm, light having the same wavelength of 532 nm as that of the stimulating ray is cut off by the filter 52b and only light in the wavelength of the fluorescence emission 45 released from Rhodamine passes through the filter 52b to be photoelectrically detected by the photomultiplier 50.

Analog data produced by photoelectrically detecting stimulated emission 45 with the photomultiplier 50 are converted by an A/D converter 53 into digital data and the digital data are fed to a data processing apparatus 54.

When a predetermined time, for example, several microseconds, has passed after the second laser stimulating ray source 22 was turned on, the control unit 70 outputs a drive stop signal to the second laser stimulating ray source 22, thereby turning it off and outputs a drive signal to the main scanning stepping motor 65, thereby moving the optical head 35 by one pitch equal to the distance between neighboring absorptive regions 4 formed in the biochemical analysis unit 1.

When the control unit 70 determines based on a detection signal indicating the position of the optical head 35 input from the linear encoder 67 that the optical head 35 has been moved by one pitch equal to the distance between neighboring absorptive regions 4 formed in the substrate 2 of the biochemical analysis unit 1 and has reached a position where a laser beam 24 can be projected onto a second absorptive region 4 next to the first absorptive region 4 formed in the substrate 2 of the biochemical analysis unit 1, it outputs a drive signal to the second laser stimulating ray source 22 to turn it on, thereby causing the laser beam 24 to excite a fluorescent substance, for example, Rhodamine, contained in the second absorptive region 4 formed in the substrate 2 of the biochemical analysis unit 1 next to the first absorptive region 4.

Similarly to the above, the second absorptive region 4 formed in the substrate 2 of the biochemical analysis unit 1 is irradiated with the laser beam 24 for a predetermined time and when fluorescence emission 45 released from the second absorptive region 4 is photoelectrically detected by the photomultiplier 50, the control unit 70 outputs a drive stop signal to the second laser stimulating ray source 21, thereby turning it off and outputs a drive signal to the main scanning stepping motor 65, thereby moving the optical head 35 by one pitch equal to the distance between neighboring absorptive regions 4 formed in the substrate 2 of the biochemical analysis unit 1.

In this manner, the on and off operation of the second laser stimulating ray source 22 is repeated in synchronism with the intermittent movement of the optical head 35 and when the control unit 70 determines based on a detection signal indicating the position of the optical head 35 input from the linear encoder 67 that the optical head 35 has been moved by one scanning line in the main scanning direction and that the absorptive regions 4 included in a first line of the absorptive regions 4 formed in the substrate 2 of the biochemical analysis unit 1 have been scanned with the laser beam 24, it outputs a drive signal to the main scanning stepping motor 65, thereby returning the optical head 35 to its original position and outputs a drive signal to the sub-scanning pulse motor 61, thereby causing it to move the movable base plate 63 by one scanning line in the sub-scanning direction.

When the control unit 70 determines based on a detection signal indicating the position of the optical head 35 input from the linear encoder 67 that the optical head 35 has been returned to its original position and determines that the movable base plate 63 has been moved by one scanning line in the sub-scanning direction, similarly to the manner in which the absorptive regions 4 included in the first line of the absorptive regions 4 formed in the substrate 2 of the biochemical analysis unit 1 were sequentially irradiated with the laser beam 24 emitted from the second laser stimulating ray source 22, the absorptive regions 4 included in a second line of the absorptive regions 4 formed in the substrate 2 of the biochemical analysis unit 1 are sequentially irradiated with the laser beam 24 emitted from the second laser stimulating ray source 22, thereby exciting Rhodamine contained in the absorptive regions 4 included in the second line and fluorescence emission 45 released from the absorptive regions 4 included in the second line is sequentially and photoelectrically detected by the photomultiplier 50.

Analog data produced by photoelectrically detecting stimulated emission 45 with the photomultiplier 50 are converted by an A/D converter 53 into digital data and the digital data are fed to a data processing apparatus 54.

When all of the absorptive regions 4 formed in the substrate 2 of the biochemical analysis unit 1 have been scanned with the laser beam 24 to excite Rhodamine contained in the absorptive regions 4 and digital data produced by photoelectrically detecting fluorescence emission 45 released from the absorptive regions 4 by the photomultiplier 50 to produce analog data and digitizing the analog data by the A/D converter 53 have been forwarded to the data processing apparatus 54, the control unit 70 outputs a drive stop signal to the second laser stimulating ray source 22, thereby turning it off.

As described above, fluorescence data recorded in a number of the absorptive regions 4 of the biochemical analysis unit 1 are read by the scanner to produce biochemical analysis data.

Chemiluminescence data of a labeling substance recorded in absorptive regions 4 formed in the biochemical analysis unit 1 are transferred onto a stimulable phosphor sheet and read by a scanner described later.

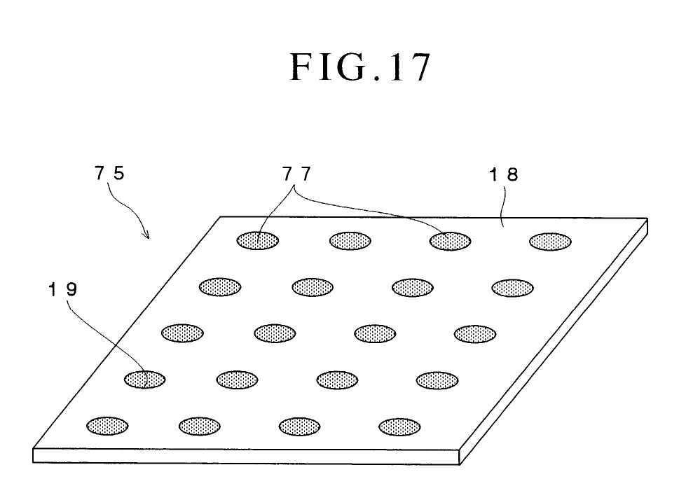

FIG. 17 is a schematic perspective view showing a stimulable phosphor sheet onto which chemiluminescence data are to be transferred.

A stimulable phosphor sheet 75 shown in FIG. 17 has the same configuration as that of the stimulable phosphor sheet 17 shown in FIG. 7 except that a number of stimulable phosphor layer regions 77 are formed by charging SrS system stimulable phosphor capable of absorbing and storing light energy in a number of the through-holes 19 formed in the support 18.

Chemiluminescence data recorded in a number of the absorptive regions 4 of the biochemical analysis unit 1 are transferred onto a number of the stimulable phosphor layer regions 77 of the stimulable phosphor sheet 75 shown in FIG. 17.

When chemiluminescence data recorded in a number of the absorptive regions 4 of the biochemical analysis unit 1 are to be transferred onto a number of the stimulable phosphor layer regions 77 of the stimulable phosphor sheet 75, a number of the absorptive regions 4 of the biochemical analysis unit 1 are first brought into contact with a chemiluminescent substrate.

As a result, chemiluminescence emission in a wavelength of visible light is selectively released from a number of the absorptive regions 4 of the biochemical analysis unit 1.

The stimulable phosphor sheet 75 is then superposed on the biochemical analysis unit 1 formed with a number of the absorptive regions 4 selectively releasing chemiluminescence emission in such a manner that a number of the stimulable phosphor layer regions 77 formed in the stimulable phosphor sheet 75 face the corresponding absorptive regions 4 formed in the biochemical analysis unit 1.

In this manner, each of the stimulable phosphor layer regions 77 formed in the support 18 of the stimulable phosphor sheet 75 is kept to face the corresponding absorptive region 4 formed in the substrate 2 of the biochemical analysis unit 1 for a predetermined time period, whereby a number of the stimulable phosphor layer regions 77 formed in the support 18 of the stimulable phosphor sheet 75 are exposed to chemiluminescence emission released from a number of the absorptive regions 4 formed in the substrate 2 of the biochemical analysis unit 1.

In this embodiment, since the substrate 2 made of stainless steel capable of attenuating light energy are present around each of the absorptive regions 4 formed in the biochemical analysis unit 1, chemiluminescence emission released from the absorptive regions 4 of the biochemical analysis unit 1 during the exposure operation can be efficiently prevented from scattering in the biochemical analysis unit 1. Further, since the support 18 of the stimulable phosphor sheet 75 is made of stainless steel capable of attenuating light energy, chemiluminescence emission released from the absorptive regions 4 of the biochemical analysis unit 1 can be efficiently prevented from scattering in the support 18 of the stimulable phosphor sheet 75 and impinging on the stimulable phosphor layer regions 77 neighboring absorptive regions 4 face.

In this manner, chemiluminescence data are recorded in a number of the stimulable phosphor layer regions 77 formed in the support 18 of the stimulable phosphor sheet 75.

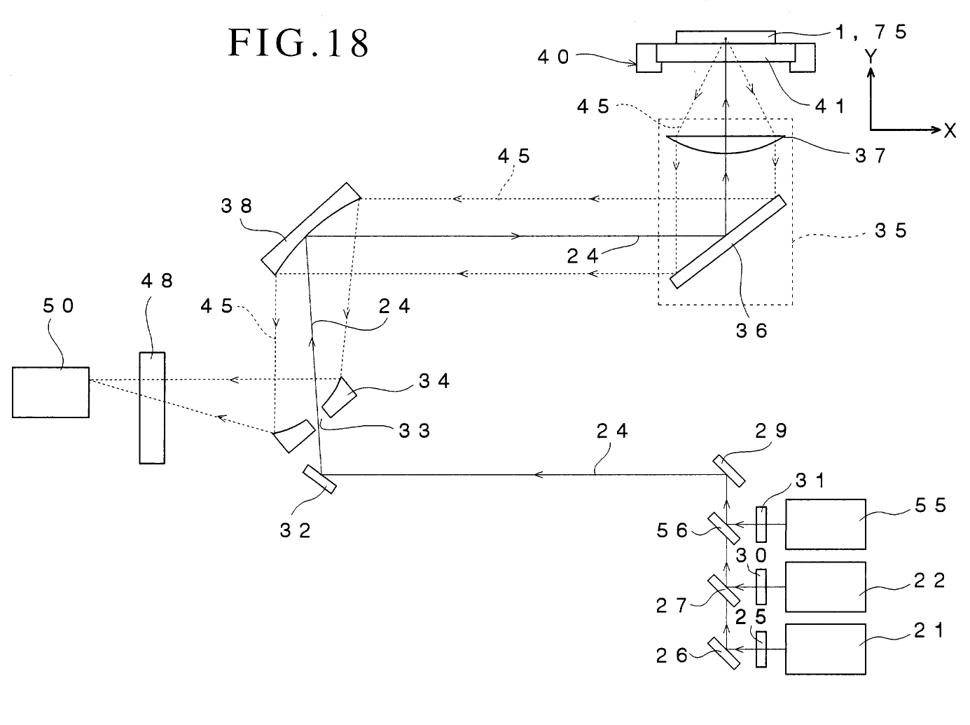

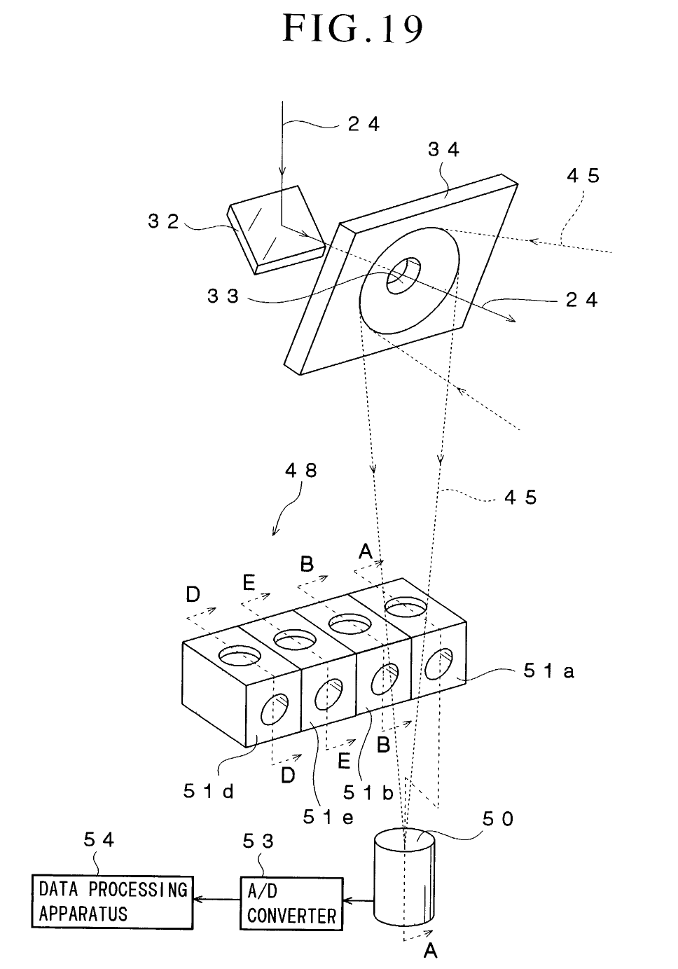

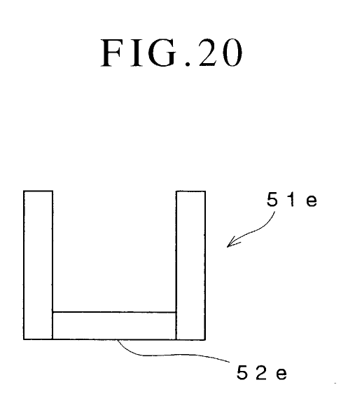

FIG. 18 is a schematic view showing a scanner for reading chemiluminescence data recorded in a number of the stimulable phosphor layer regions 77 formed in the support 18 of the stimulable phosphor sheet 75 and producing biochemical analysis data. FIG. 19 is a schematic perspective view showing details in the vicinity of a photomultiplier of a scanner shown in FIG. 18 and FIG. 20 is a schematic cross-sectional view taken along a line E-E in FIG. 19.

The scanner shown in FIGS. 18 to 20 has the same configuration as that of the first scanner shown in FIGS. 9 to 16 except that it includes a fourth laser stimulating ray source 55 for emitting a laser beam 24 having a wavelength of 980 nm which can effectively stimulate SrS system stimulable phosphor instead of the third laser stimulating ray source 23 for emitting a laser beam 24 having a wavelength of 473 nm, includes a filter member 51e provided with a filter having a property of transmitting only light having a wavelength corresponding to that of stimulated emission emitted from stimulable phosphor and cutting off light having a wavelength of 980 nm, and includes a third dichroic mirror 56 for transmitting light having a wavelength equal to and shorter than 640 nm but reflecting light having a wavelength of 980 nm instead of the second dichroic mirror 28 for transmitting light having a wavelength equal to and longer than 532 nm but reflecting light having a wavelength of 473 nm.

The thus constituted scanner reads chemiluminescence data recorded in a number of the stimulable phosphor layer regions 77 of the stimulable phosphor sheet 75 and produces biochemical analysis data in the following manner.

A stimulable phosphor sheet 75 is first set on the glass plate 41 of the stage 40 by a user.

An instruction signal indicating that chemiluminescence data recorded in the stimulable phosphor layer 77 formed in the stimulable phosphor sheet 75 are to be read is then input through the keyboard 71.

The instruction signal input through the keyboard 71 is input to the control unit 70 and the control unit 70 outputs a drive signal to the filter unit motor 72 in accordance with the instruction signal, thereby moving the filter unit 48 so as to locate the filter member 51e provided with the filter 52e having a property of transmitting only light having a wavelength corresponding to that of stimulated emission emitted from the stimulable phosphor layer regions 77 and cutting off light having a wavelength of 980 nm in the optical path of stimulated emission 45.

The control unit 70 further outputs a drive signal to the main scanning stepping motor 65 to move the optical head 35 in the main scanning direction and when it determines based on a detection signal indicating the position of the optical head 35 input from the linear encoder 67 that the optical head 35 has reached a position where a laser beam 24 can be projected onto a first stimulable phosphor layer region 77 among a number of the stimulable phosphor layer regions 77 formed in the support 18 of the stimulable phosphor sheet 75, it outputs a drive stop signal to the main scanning stepping motor 65 and a drive signal to the fourth stimulating ray source 55, thereby actuating it to emit a laser beam 24 having a wavelength of 980 nm.

A laser beam 24 emitted from the fourth laser stimulating ray source 55 passes through a collimator lens 31, thereby being made a parallel beam, and is reflected by the third dichroic mirror 56, thereby changing its direction by 90 degrees. The laser beam 24 then advances to the mirror 29.

The laser beam 24 advancing to the mirror 29 is reflected by the mirror 29 and advances to the mirror 32 to be reflected thereby.

The laser beam 24 reflected by the mirror 32 passes through the hole 33 of the perforated mirror 34 and advances to the concave mirror 38.

The laser beam 24 advancing to the concave mirror 38 is reflected by the concave mirror 38 and enters the optical head 35.

The laser beam 24 entering the optical head 35 is reflected by the mirror 36 and condensed by the aspherical lens 37 onto the first stimulable phosphor layer region 77 of the stimulable phosphor sheet 77 placed on the glass plate 41 of a stage 40.

In this embodiment, since each of the stimulable phosphor layer regions 77 of the stimulable phosphor sheet 75 is formed by charging stimulable phosphor in the through-hole 19 formed in the support 18 made of stainless steel capable of attenuating light energy, it is possible to effectively prevent the laser beam 24 from scattering in each of the stimulable phosphor layer regions 77 and entering the neighboring stimulable phosphor layer regions 77 to excite stimulable phosphor contained in the neighboring stimulable phosphor layer regions 77.

When the laser beam 24 impinges onto the first stimulable phosphor layer region 77 formed in the support 18 of the stimulable phosphor sheet 75, stimulable phosphor contained in the first stimulable phosphor layer region 77 formed in the support 18 of the stimulable phosphor sheet 75 is excited by the laser beam 24, thereby releasing stimulated emission 45 from the first stimulable phosphor layer region 77.

The stimulated emission 45 released from the first stimulable phosphor layer region 77 of the stimulable phosphor sheet 75 is condensed onto the mirror 36 by the aspherical lens 37 provided in the optical head 35 and reflected by the mirror 36 on the side of the optical path of the laser beam 24, thereby being made a parallel beam to advance to the concave mirror 38.

The stimulated emission 45 advancing to the concave mirror 38 is reflected by the concave mirror 38 and advances to the perforated mirror 34.

As shown in FIG. 19, the stimulated emission 45 advancing to the perforated mirror 34 is reflected downward by the perforated mirror 34 formed as a concave mirror and advances to the filter 52e of the filter unit 48.

Since the filter 52e has a property of transmitting only light having a wavelength corresponding to that of stimulated emission emitted from stimulable phosphor and cutting off light having a wavelength of 980 nm, light having a wavelength of 980 nm corresponding to that of the stimulating ray is cut off by the filter 52e and only light having a wavelength corresponding to that of stimulated emission passes through the filter 52e to be photoelectrically detected by the photomultiplier 50.

Analog data produced by photoelectrically detecting stimulated emission 45 with the photomultiplier 50 are converted by an A/D converter 53 into digital data and the digital data are fed to a data processing apparatus 54.

When a predetermined time, for example, several microseconds, has passed after the fourth stimulating ray source 55 was turned on, the control unit 70 outputs a drive stop signal to the fourth stimulating ray source 55, thereby turning it off and outputs a drive signal to the main scanning stepping motor 65, thereby moving the optical head 35 by one pitch equal to the distance between neighboring stimulable phosphor layer regions 77 formed in the support 18 of the stimulable phosphor sheet 75.

When the control unit 70 determines based on a detection signal indicating the position of the optical head 35 input from the linear encoder 67 that the optical head 35 has been moved by one pitch equal to the distance between neighboring stimulable phosphor layer regions 77, it outputs a drive signal to the fourth stimulating ray source 55 to turn it on, thereby causing the laser beam 24 to excite stimulable phosphor contained in a second stimulable phosphor layer region 77 formed in the support 18 of the stimulable phosphor sheet 75 next to the first stimulable phosphor layer region 77.

Similarly to the above, the second stimulable phosphor layer region 77 formed in the support 18 of the stimulable phosphor sheet 75 is irradiated with the laser beam 24 for a predetermined time and when stimulated emission 45 released from the second stimulable phosphor layer region 77 is photoelectrically detected by the photomultiplier 50, the control unit 70 outputs a drive stop signal to the fourth stimulating ray source 55, thereby turning it off and outputs a drive signal to the main scanning stepping motor 65, thereby moving the optical head 35 by one pitch equal to the distance between neighboring stimulable phosphor layer regions 77.

In this manner, the on and off operation of the fourth stimulating ray source 55 is repeated in synchronism with the intermittent movement of the optical head 35 and when the control unit 70 determines based on a detection signal indicating the position of the optical head 35 input from the linear encoder 67 that the optical head 35 has been moved by one scanning line in the main scanning direction and that the stimulable phosphor layer regions 77 included in a first line of the stimulable phosphor layer regions 77 formed in the support 18 of the stimulable phosphor sheet 75 have been scanned with the laser beam 24, it outputs a drive signal to the main scanning stepping motor 65, thereby returning the optical head 35 to its original position and outputs a drive signal to the sub-scanning pulse motor 61, thereby causing it to move the movable base plate 63 by one scanning line in the sub-scanning direction.

When the control unit 70 determines based on a detection signal indicating the position of the optical head 35 input from the linear encoder 67 that the optical head 35 has been returned to its original position and determines that the movable base plate 63 has been moved by one scanning line in the sub-scanning direction, similarly to the manner in which the stimulable phosphor layer regions 77 included in the first line of the stimulable phosphor layer regions 77 formed in the support 18 of the stimulable phosphor sheet 75 were sequentially irradiated with the laser beam 24 emitted from the fourth laser stimulating ray source 55, the stimulable phosphor layer regions 77 included in a second line of the stimulable phosphor layer regions 77 formed in the support 18 of the stimulable phosphor sheet 75 are sequentially irradiated with the laser beam 24 emitted from the fourth laser stimulating ray source 55, thereby exciting stimulable phosphor contained in the stimulable phosphor layer regions 77 included in the second line and stimulated emission 45 released from the stimulable phosphor layer regions 77 included in the second line is sequentially and photoelectrically detected by the photomultiplier 50.

Analog data produced by photoelectrically detecting stimulated emission 45 with the photomultiplier 50 are converted by an A/D converter 53 into digital data and the digital data are fed to a data processing apparatus 54.

When all of the stimulable phosphor layer regions 77 formed in the support 18 of the stimulable phosphor sheet 75 have been scanned with the laser beam 24 to excite stimulable phosphor contained in the stimulable phosphor layer regions 77 and digital data produced by photoelectrically detecting stimulated emission 45 released from the stimulable phosphor layer regions 77 by the photomultiplier 50 to produce analog data and digitizing the analog data by the A/D converter 53 have been forwarded to the data processing apparatus 54, the control unit 70 outputs a drive stop signal to the fourth laser stimulating ray source 55, thereby turning it off.

As described above, chemiluminescence data recorded in a number of the stimulable phosphor layer regions 77 of the stimulable phosphor sheet 75 are read by the scanner to produce biochemical analysis data.

According to this embodiment, since the mixed solution of the hybridization buffer and the probe solution is forcibly fed into the cartridge 7 by the pump 15 through a number the absorptive regions 4 formed in the substrate 2 of the biochemical analysis unit 1 held in the cartridge 7, thereby performing hybridization, it is possible to markedly increase the moving rate of a substance derived from a living organism through the absorptive regions 4 of the biochemical analysis unit 1 in comparison with the case where a substance derived from a living organism and contained in the mixed solution of the hybridization buffer and the probe solution is moved only by convection or diffusion to be hybridized with specific binding substances absorbed in a number of the absorptive regions 4 of the biochemical analysis unit 1 and, therefore, the reaction rate of hybridization can be markedly improved. Further, since it is possible to markedly improve the possibility of a substance derived from a living organism and contained in mixed solution of the hybridization buffer and the probe solution associating with specific binding substances absorbed in deep portions of a number of the absorptive regions 4 formed in the substrate 2 of the biochemical analysis unit 1, a substance derived from a living organism and contained in mixed solution of the hybridization buffer and the probe solution can be hybridized with specific binding substances absorbed in a number of the absorptive regions 4 formed in the substrate 2 of the biochemical analysis unit 1 in a desired manner.

Further, according to this embodiment, since the mixed solution of the hybridization buffer and the probe solution is circulated by the pump 15 into the cartridge 7 via the solution circulation pipe 14 and forcibly fed into the cartridge 7 so as to pass through a number the absorptive regions 4 formed in the substrate 2 of the biochemical analysis unit 1 held in the cartridge 7 repeatedly, it is possible to much more improve the possibility of a substance derived from a living organism and contained in mixed solution of the hybridization buffer and the probe solution associating with specific binding substances absorbed in deep portions of a number of the absorptive regions 4 formed in the substrate 2 of the biochemical analysis unit 1 and therefore, a substance derived from a living organism and contained in mixed solution of the hybridization buffer and the probe solution can be hybridized with specific binding substances absorbed in a number of the absorptive regions 4 formed in the substrate 2 of the biochemical analysis unit 1 in a desired manner.

Moreover, according to this embodiment, since the antibody solution is forcibly fed into the cartridge 7 by the pump 15 so as to pass through a number the absorptive regions 4 formed in the substrate 2 of the biochemical analysis unit 1 held in the cartridge 7, thereby performing an antigen-antibody reaction, it is possible to markedly increase the moving rate of an antibody through the absorptive regions 4 of the biochemical analysis unit 1 and, therefore, the reaction rate of an antigen-antibody reaction can be markedly improved. Further, since it is possible to markedly improve the possibility of an antibody for the hapten contained in the antibody solution associating with the hapten labeling a substance derived from a living organism selectively hybridized with specific binding substances absorbed in deep portions of a number of the absorptive regions 4 formed in the substrate 2 of the biochemical analysis unit 1, an antibody for the hapten contained in the antibody solution can be associated with the hapten labeling a substance derived from a living organism selectively hybridized with specific binding substances absorbed in a number of the absorptive regions 4 formed in the substrate 2 of the biochemical analysis unit 1 in a desired manner.

Further, according to this embodiment, since the antibody solution is circulated by the pump 15 into the cartridge 7 via the solution circulation pipe 14 and forcibly fed into the cartridge 7 so as to pass through a number the absorptive regions 4 formed in the substrate 2 of the biochemical analysis unit 1 held in the cartridge 7 repeatedly, it is possible to much more improve the possibility of an antibody for the hapten contained in the antibody solution associating with the hapten labeling a substance derived from a living organism selectively hybridized with specific binding substances absorbed in deep portions of a number of the absorptive regions 4 formed in the substrate 2 of the biochemical analysis unit 1 and therefore, an antibody for the hapten contained in the antibody solution can be associated with the hapten labeling a substance derived from a living organism selectively hybridized with specific binding substances absorbed in a number of the absorptive regions 4 formed in the substrate 2 of the biochemical analysis unit 1 in a desired manner.

Moreover, according to this embodiment, since the cleaning solution is forcibly fed into the cartridge 7 by the pump 15 so as to pass through a number the absorptive regions 4 formed in the substrate 2 of the biochemical analysis unit 1 held in the cartridge 7, thereby performing the cleaning of the absorptive regions 4 of the biochemical analysis unit 1, even if a substance derived from a living organism which should not be hybridized with specific binding substances absorbed in the absorptive regions 4 formed in the substrate 2 of the biochemical analysis unit 1 has been bonded with the absorptive regions 4 during the process of hybridization, it is possible to very efficiently peel off and remove the substance derived from a living organism which should not be hybridized with specific binding substances absorbed in the absorptive regions 4 from a number of the absorptive regions 4 of the biochemical analysis unit 1 and even if an antibody which should not be bonded with the hapten labeling a substance derived from a living body and selectively hybridized with specific binding substances absorbed in the absorptive regions 4 of the biochemical analysis unit 1 has been bonded with the absorptive regions 4 formed in the substrate 2 of the biochemical analysis unit 1, it is possible to very efficiently peel off and remove the antibody which should not be bonded with the hapten from a number of the absorptive regions 4 of the biochemical analysis unit 1. Therefore, the efficiency of cleaning operation can be markedly improved.

Furthermore, according to this embodiment, since the mixed solution of the hybridization buffer and the probe solution is forcibly fed into the cartridge 7 by the pump 15 so as to pass through a number the absorptive regions 4 formed in the substrate 2 of the biochemical analysis unit 1 held in the cartridge 7, thereby performing hybridization and the cleaning solution is forcibly fed into the cartridge 7 by the pump 15 so as to pass through a number the absorptive regions 4 formed in the substrate 2 of the biochemical analysis unit 1 held in the cartridge 7, thereby performing the cleaning of the absorptive regions 4 of the biochemical analysis unit 1, even if a different experimenter performs hybridization, it is possible to reliably prevent different results from being obtained and the repeatability of hybridization can be markedly improved.

Moreover, according to this embodiment, since the antibody solution is forcibly fed into the cartridge 7 by the pump 15 so as to pass through a number the absorptive regions 4 formed in the substrate 2 of the biochemical analysis unit 1 held in the cartridge 7, thereby performing an antigen-antibody reaction and the cleaning solution is forcibly fed into the cartridge 7 by the pump 15 so as to pass through a number the absorptive regions 4 formed in the substrate 2 of the biochemical analysis unit 1 held in the cartridge 7, thereby performing the cleaning of the absorptive regions 4 of the biochemical analysis unit 1, even if a different experimenter performs an antigen-antibody reaction, it is possible to reliably prevent different results from being obtained and the repeatability of an antigen-antibody reaction can be markedly improved.

Further, according to this embodiment, since a number of the absorptive regions 4 of the biochemical analysis unit 1 are formed by charging nylon-6 in a number of the through-holes 3 formed in the substrate 2 made of stainless steel, the hybridization buffer, the mixed solution of the hybridization buffer and the probe solution and the cleaning solution are forcibly fed to the biochemical analysis unit 1 so as to cut through only a number of the absorptive regions 4 formed in the substrate 2 of the biochemical analysis unit 1 and, therefore, the efficiency of hybridization, the efficiency of an antigen-antibody reaction and the efficiency of the cleaning operation can be markedly improved.

Furthermore, according to this embodiment, since the mixed solution of the hybridization buffer and the probe solution is forcibly fed to only the absorptive regions 4 of the biochemical analysis unit 1, it is possible to reliably prevent a substance derived from a living organism from adhering to portions of the substrate 2 of the biochemical analysis unit 1 other than the absorptive regions 4. Therefore, since it is sufficient to feed a cleaning solution to only the absorptive regions 4 of the biochemical analysis unit 1, thereby cleaning them, the efficiency of the cleaning operation can be improved.

Moreover, according to this embodiment, since the antibody solution is forcibly fed to only the absorptive regions 4 of the biochemical analysis unit 1, it is possible to reliably prevent an antibody from adhering to portions of the substrate 2 of the biochemical analysis unit 1 other than the absorptive regions 4. Therefore, since it is sufficient to feed a cleaning solution to only the absorptive regions 4 of the biochemical analysis unit 1, thereby cleaning them, the efficiency of the cleaning operation can be improved.

Further, according to this embodiment, since each of the second branch passages 8aa, 8ab, . . . , 8am; 8ba, 8bb, . . . , 8bm; 8ca, 8cb, . . . , 8cm; . . . ; 8na, 8nb, . . . , 8nm and each of the third branch passages 9aa, 9ab, . . . , 9am; 9ba, 9bb, . . . , 9bm; 9ca, 9cb, . . . , 9cm; . . . ; 9na, 9nb, . . . , 9nm are formed so as to have the same size as that of each of the absorptive regions 4 formed in the substrate 2 of the biochemical analysis unit 1 and each of the absorptive regions 4 formed in the substrate 2 of the biochemical analysis unit 1 has a size of about 0.01 mm², the reaction occurs within a micro-area of about 0.01 mm². Therefore, since the reaction can be facilitated in accordance with the principle of a micro-reactor, the efficiency of the hybridization, the efficiency of the antigen-antibody reaction and the efficiency of the cleaning operation can be markedly improved.

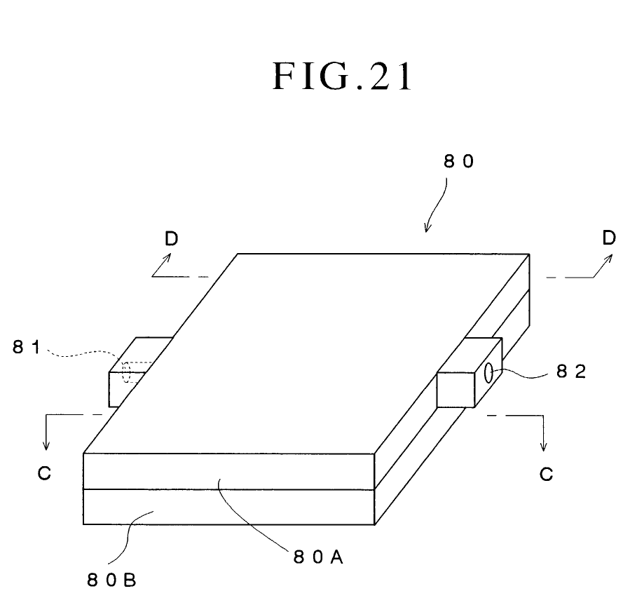

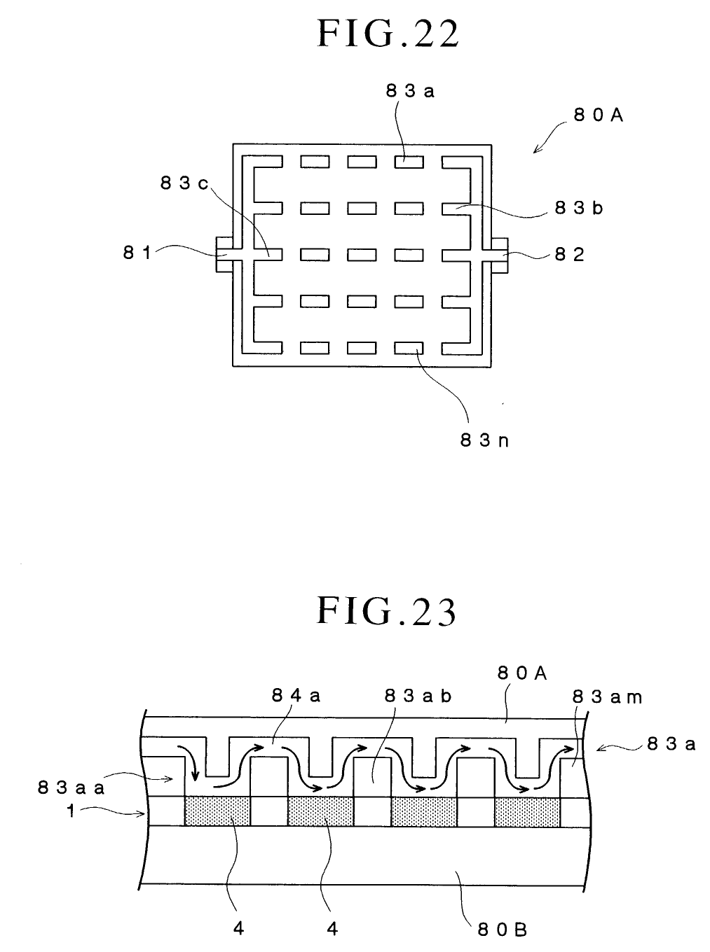

FIG. 21 is a schematic perspective view showing a cartridge for a biochemical analysis unit which is another preferred embodiment of the present invention and FIG. 22 is a schematic cross sectional view taken along a line C-C in FIG. 21.

As shown in FIG. 21, the cartridge 80 for a biochemical analysis unit according to this embodiment includes an upper half portion 80A and a lower half portion 80B and the biochemical analysis unit 1 is held between the upper half portion 80A and the lower half portion 80B.

A solution feed passage 81 is formed at the substantial center portion of one side surface of the upper half portion 80A for feeding a solution into the cartridge 80 and a solution discharge passage 82 is formed at the substantial center portion of the other surface of the upper half portion 80A for discharging a solution from the cartridge 80.

As shown in FIG. 22, the solution feed passage 81 branches into n branch passages 83a, 83b, 83c, 83d, . . . , 83n correspondingly to the number n of columns of the absorptive regions 4 formed in the substrate 2 of the biochemical analysis unit.

FIG. 23 is a schematic cross sectional view taken along a line D-D in FIG. 21.

As shown in FIG. 23, the branch passage 83a includes a parallel passage 84a extending in parallel with the surface of the biochemical analysis unit 1 held in the cartridge 80 and folded passages 83aa, 83ab, . . . , 83am each of which are bent downward in a substantially perpendicular direction at a position corresponding to the upstream portion of an absorptive region 4 of the biochemical analysis unit 1 held in the cartridge 80 and bent upward in a substantially perpendicular direction at a position corresponding to the downstream portion of the absorptive region 4 with respect to the flowing direction of a solution so that a solution flowing in the branch passage 83a can come into contact with only the absorptive regions 4 of the biochemical analysis unit 1 corresponding to the branch passages 83a.

Although not shown in FIG. 23, each of the branch passages 83b, 83c, 83n similarly includes a parallel passage 84b, 84c, 84n extending in parallel with the surface of the biochemical analysis unit 1 held in the cartridge 80 and folded passages 83ba, 83bb, . . . , 83bm; 83ca, 83cb, 83cm, . . . , 83na, 83nb, . . . , 83nm each of which is bent downward in a substantially perpendicular direction at a position corresponding to the upstream portion of an absorptive region 4 of the biochemical analysis unit 1 held in the cartridge 80 and bent upward in a substantially perpendicular direction at a position corresponding to the downstream portion of the absorptive region 4 with respect to the flowing direction of a solution so that a solution flowing therein can come into contact with only the absorptive regions 4 of the biochemical analysis unit 1 corresponding thereto.

Therefore, each of the branch passages 83a, 83b, 83c, 83d, . . . , 83n includes the folded passages 83aa, 83ab, . . . , 83am; 83ba, 83bb, . . . , 83bm; 83ca, 83cb, . . . , 83cm; . . . , 83na, 83nb, 83nm whose number is equal to that of the absorptive regions 4 included in one line of the absorptive regions 4 formed in the substrate 2 of the biochemical analysis unit 1.

In this embodiment, each of the folded passages 83aa, 83ab, . . . , 83am; 83ba, 83bb, . . . , 83bm; 83ca, 83cb, . . . , 83cm; . . . , 83na, 83nb, . . . , 83nm is formed so as to have the same size as that of the absorptive region 4 of the biochemical analysis unit 1 so that the length thereof is equal to or shorter than 0.5 mm, preferably, 0.1 mm.

When a substance derived from a living organism and labeled with a labeling substance is to be selectively hybridized with specific binding substances fixed in a number of the absorptive regions 4 of the biochemical analysis unit 1, the thus constituted cartridge 80 for a biochemical analysis unit holding the biochemical analysis unit 1 therein is first set on the support base 7C of the apparatus for a receptor-ligand association reaction. One end portion of the solution circulating pipe 14 of the apparatus for a receptor-ligand association reaction is then connected to the solution feed passage 81 of the cartridge 80 and the other end portion of the solution circulating pipe 14 is connected to the solution discharge passage 82 of the cartridge 80.

When hybridization is to be performed, a hybridization buffer is first prepared and accommodated in the hybridization buffer tank 10.

Then, the change-over valve 11b provided in the probe solution feed pipe 11a, the change-over valve 12b provided in the antibody solution feed pipe 12a and the change-over valve 13b provided in the cleaning solution feed pipe 13a are located at their second positions where the atmosphere and the solution circulation pipe 14 communicate with each other and the change-over valve 16a provided at the bifurcated portion of the solution circulation pipe 14 and the solution discharge pipe 16 is located at its first position.

When the change-over valve 10b provided in the hybridization buffer feed pipe 10a has been located at its first position where the hybridization buffer feed pipe 10a and the solution circulation pipe 14 communicate with each other, the pump 15 is driven.

As a result, a hybridization buffer accommodated in the hybridization buffer tank 10 is fed into the solution feed passage 81 formed in the cartridge 80 via the hybridization buffer feed pipe 10a and the solution circulation pipe 14.

In this embodiment, since the solution feed passage 81 branches into the n branch passages 83a, 83b, 83c, . . . , 83n whose number is equal to the number of columns of the absorptive regions 4 of the biochemical analysis unit 1, the hybridization buffer flows into the n branch passages 83a, 83b, 83c, . . . , 83n from the solution feed passage 81.

The hybridization buffer flowing in the branch passages 83a, 83b, 83c, . . . , 83n flows toward a merged portion with the solution discharge passage 82 of the cartridge 80.

When the hybridization buffer has reached the folded passages 83aa, 83ab, . . . , 83am; 83ba, 83bb, . . . , 83bm; 83ca, 83cb, . . . , 83cm; . . . , 83na, 83nb, . . . , 83nm formed at corresponding absorptive regions 4 of the biochemical analysis unit 1, the hybridization buffer turns downward as shown in FIG. 23 and comes into contact with the corresponding absorptive region 4 of the biochemical analysis unit 1. The hybridization buffer then turns upward and flows toward the merged portion with the solution discharge passage 82 of the cartridge 80.

The contact time between the hybridization buffer and the corresponding absorptive region 4 can be controlled by adjusting the gap between the individual absorptive regions 4 formed in the substrate 2 of the biochemical analysis unit 1 and the upper half portion 80A of the cartridge 80.

In this manner, pre-hybridization is performed.

The hybridization buffer is fed from the branch passages 83a, 83b, 83c, . . . , 83n to the solution circulation passage 14 of the apparatus for a receptor-ligand association reaction via the merged portion with the solution discharge passage 82 of the cartridge 80 and recycled into the cartridge 80.

In this manner, when an inner space of the cartridge 80 and the solution circulation pipe 14 has been filled with the hybridization buffer, the change-over valve 10b provided in the hybridization buffer feed pipe 10a is located at its third position where communication between the hybridization buffer tank 10 and the atmosphere, and the solution circulation pipe 14 is shut off and the change-over valve 11b provided in the probe solution feed pipe 11a, the change-over valve 12b provided in the antibody solution feed pipe 12a and the change-over valve 13b provided in the cleaning solution feed pipe 13a are located at their third positions.

On the other hand, the pump 15 continues to be driven and as a result, the hybridization buffer filling the inner space of the cartridge 80 and the solution circulation pipe 14 is circulated through the cartridge 80 and the solution circulation pipe 14, whereby the hybridization buffer is forcibly moved so as to come into contact with a number of the absorptive regions 4 of the biochemical analysis unit 1 held in the cartridge 80.

When a first predetermined time period has passed, the pump 15 is stopped and pre-hybridization is completed.

Then, a probe solution is prepared and accommodated in the probe solution chip 11.

Similarly to the previous embodiment, in this embodiment, a probe solution containing a substance derived from a living organism and labeled with a radioactive labeling substance, a substance derived from a living organism and labeled with a fluorescent substance such as a fluorescent dye and a substance derived from a living organism and labeled with hapten such as digoxigenin is prepared and accommodated in the probe solution chip 11.

When the probe solution has been accommodated in the probe solution chip 11, the change-over valve 10b provided in the hybridization buffer feed pipe 10a, the change-over valve 12b provided in the antibody solution feed pipe 12a and the change-over valve 13b provided in the cleaning solution feed pipe 13a are located at their second positions where the atmosphere and the solution circulation pipe 14 communicate with each other and the change-over valve 16a provided at the bifurcated portion of the solution circulation pipe 14 and the solution discharge pipe 16 is located at its first position.

Then, the change-over valve 11b provided in the probe solution feed pipe 11a is located at its first position where the probe solution feed pipe 11a and the solution circulation pipe 14 communicate with each other and the pump 15 is driven.

As a result, a probe solution accommodated in the probe solution chip 11 is fed into the solution circulation pipe 14 via the probe solution feed pipe 11a and mixed with the hybridization buffer filling the inner space of the cartridge 80 and the solution circulation pipe 14.

When a predetermined amount of the probe solution has been fed from the probe solution chip 11, the change-over valve 11b provided in the probe solution feed pipe 11a is located at its third position where communication between the probe solution tank 11 and the atmosphere, and the solution circulation pipe 14 is shut off and the change-over valve 10b provided in the hybridization buffer feed pipe 10a, the change-over valve 12b provided in the antibody solution feed pipe 12a and the change-over valve 13b provided in the cleaning solution feed pipe 13a are located at their third positions.

On the other hand, the pump 15 continues to be driven and therefore, the mixed solution produced by mixing the probe solution with the hybridization buffer filling the inner space of the cartridge 80 and the solution circulation pipe 14 is fed into the solution feed passage 81 formed in the cartridge 80 from the solution circulation pipe 14.

Since the solution feed passage 81 branches into the n branch passages 83a, 83b, 83c, . . . , 83n whose number is equal to the number of columns of the absorptive regions 4 of the biochemical analysis unit 1, the mixed solution of the hybridization buffer and the probe solution flows into the n branch passages 83a, 83b, 83c, . . . , 83n from the solution feed passage 81.

When the mixed solution of the hybridization buffer and the probe solution flowing in the branch passages 83a, 83b, 83c, . . . , 83n has reached the folded passages 83aa, 83ab, . . . , 83am; 83ba, 83bb, . . . , 83bm; 83ca, 83cb, . . . , 83cm; . . . , 83na, 83nb, . . . , 83nm formed at corresponding absorptive regions 4 of the biochemical analysis unit 1, the mixed solution of the hybridization buffer and the probe solution turns downward as shown in FIG. 23 and comes into contact with the corresponding absorptive region 4 of the biochemical analysis unit 1. The mixed solution of the hybridization buffer and the probe solution then turns upward and flows toward the merged portion with the solution discharge passage 82 of the cartridge 80.

As a result, a substance derived from a living organism and contained in the mixed solution of the hybridization buffer and the probe solution selectively hybridizes with specific binding substances absorbed in a number of the absorptive regions 4 formed in the substrate 2 of the biochemical analysis unit 1 held in the cartridge 80.

The mixed solution of the hybridization buffer and the probe solution is fed from the branch passages 83a, 83b, 83c, . . . , 83n to the solution circulation passage 14 of the apparatus for a receptor-ligand association reaction via the merged portion with the solution discharge passage 82 of the cartridge 80 and recycled into the cartridge 80.

In this embodiment, since the mixed solution of the hybridization buffer and the probe solution is forcibly fed to only the individual absorptive regions 4 of the biochemical analysis unit 1 held in the cartridge 80 repeatedly in this manner by the folded passages 83aa, 83ab, . . . , 83am; 83ba, 83bb, . . . , 83bm; 83ca, 83cb, . . . , 83cm; . . . , 83na, 83nb, . . . , 83nm, thereby being brought into contact with the individual absorptive regions 4, it is possible to extremely efficiently hybridize a substance derived from a living organism and contained in the mixed solution of the hybridization buffer and the probe solution with specific binding substances fixed in a number of the absorptive regions 4 of the biochemical analysis unit 1 in comparison with the case of moving a substance derived from a living organism and contained in the mixed solution of the hybridization buffer and the probe solution by convection or diffusion and hybridizing it with specific binding substances fixed in a number of the absorptive regions 4 of the, biochemical analysis unit 1.

The contact time between the mixed solution of the hybridization buffer and the probe solution and the corresponding absorptive region 4 can be controlled by adjusting a gap between the individual absorptive regions 4 formed in the substrate 2 of the biochemical analysis unit 1 and the upper half portion 80A of the cartridge 80.

When a second predetermined time period has passed, the pump 15 is stopped and hybridization is completed.

The change-over valve 11b provided in the probe solution feed pipe 11a is then located at its second position where the atmosphere and the solution circulation pipe 14 communicate with each other and the change-over valve 16a provided at the bifurcated portion of the solution circulation pipe 14 and the solution discharge pipe 16 is located at its second position where the solution circulation pipe 14 and the solution discharge pipe 16 communicate with each other. The pump 15 is then driven.

As a result, the mixed solution of the hybridization buffer and the probe solution filling the inner space of the cartridge 80 and the solution circulation pipe 14 is discharged through the solution discharge pipe 16.

When the mixed solution of the hybridization buffer and the probe solution filling the inner space of the cartridge 80 and the solution circulation pipe 14 has been discharged through the solution discharge pipe 16, the change-over valve 16a provided at the bifurcated portion of the solution circulation pipe 14 and the solution discharge pipe 16 is located at its first position and the change-over valve 10b provided in the hybridization buffer feed pipe 10a, the change-over valve 11b provided in the probe solution feed pipe 11a and the change-over valve 12b provided in the antibody solution feed pipe 12a are located at their second positions where the atmosphere and the solution circulation pipe 14 communicate with each other.

The change-over valve 13b provided in the cleaning solution feed pipe 13a is then located at its first position where the cleaning solution feed pipe 13a and the solution circulation pipe 14 communicate with each other and the pump 15 is driven, thereby feeding a cleaning solution accommodated in the cleaning solution tank 13 into the solution circulation pipe 14 via the cleaning solution feed pipe 13a.

Since the solution feed passage 81 branches into the n branch passages 83a, 83b, 83c, . . . , 83n whose number is equal to the number of columns of the absorptive regions 4 of the biochemical analysis unit 1, the cleaning solution flows into the n branch passages 83a, 83b, 83c, . . . , 83n from the solution feed passage 81.

When the cleaning solution flowing in the branch passages 83a, 83b, 83c, . . . , 83n has reached the folded passages 83aa, 83ab, . . . , 83am; 83ba, 83bb, . . . , 83bm; 83ca, 83cb, . . . , 83cm; . . . , 83na, 83nb, . . . , 83nm each being formed at the corresponding absorptive region 4 of the biochemical analysis unit 1, the cleaning solution turns downward as shown in FIG. 23 and comes into contact with the corresponding absorptive region 4 of the biochemical analysis unit 1. The cleaning solution then turns upward and flows toward the merged portion with the solution discharge passage 82 of the cartridge 80.

In this manner, the cleaning operation of a number of the absorptive regions 4 of the biochemical analysis unit 1 is performed.

The cleaning solution is fed from the branch passages 83a, 83b, 83c, . . . , 83n to the solution circulation passage 14 of the apparatus for a receptor-ligand association reaction via the merged portion with the solution discharge passage 82 of the cartridge 80 and recycled into the cartridge 80.

In this manner, when an inner space of the cartridge 80 and the solution circulation pipe 14 has been filled with the cleaning solution, the change-over valve 13b provided in the cleaning solution feed pipe 13a is located at its third position where communication between the cleaning solution tank 13 and the atmosphere, and the solution circulation pipe 14 is shut off and the change-over valve 10b provided in the hybridization buffer feed pipe 10a, the change-over valve 11b provided in the probe solution feed pipe 11a and the change-over valve 12b provided in the antibody solution feed pipe 12a are located at their third positions.

On the other hand, the pump 15 continues to be driven and as a result, the cleaning solution filling the inner space of the cartridge 80 and the solution circulation pipe 14 is circulated through the cartridge 80 and the solution circulation pipe 14, whereby the cleaning solution is forcibly moved so as to come into contact with a number of the absorptive regions 4 of the biochemical analysis unit 1 held in the cartridge 80.

In this embodiment, since the cleaning solution is forcibly fed to only the individual absorptive regions 4 of the biochemical analysis unit 1 held in the cartridge 80 repeatedly in this manner by the folded passages 83aa, 83ab, . . . , 83am; 83ba, 83bb, . . . , 83bm; 83ca, 83cb, . . . , 83cm; . . . , 83na, 83nb, . . . , 83nm, thereby being brought into contact with the individual absorptive regions 4, it is possible to extremely efficiently clean a number of the absorptive regions 4 of the biochemical analysis unit 1 with the cleaning solution in comparison with the case of moving a cleaning solution by convection or diffusion and cleaning a number of the absorptive regions 4 of the biochemical analysis unit 1 therewith. Therefore, even in the case where a substance derived from a living organism which should not be hybridized with specific binding substances fixed in the absorptive regions 4 formed in the substrate 2 of the biochemical analysis unit 1 has been bonded with the absorptive regions 4 during the process of hybridization, since it is possible to very efficiently peel off and remove the substance derived from a living organism which should not be hybridized with specific binding substances fixed in the absorptive regions 4 from a number of the absorptive regions 4 of the biochemical analysis unit 1, it is possible to bind a substance derived from a living organism which is to be hybridized with specific binding substances fixed in a number of the absorptive regions 4 of the biochemical analysis unit 1 therewith in a desired manner.

When a third predetermined time period has passed, the pump 15 is stopped and the cleaning operation is completed.

The change-over valve 13b provided in the cleaning solution feed pipe 13a is then located at its second position where the atmosphere and the solution circulation pipe 14 communicate with each other and the change-over valve 16a provided at the bifurcated portion of the solution circulation pipe 14 and the solution discharge pipe 16 is located at its second position where the solution circulation pipe 14 and the solution discharge pipe 16 communicate with each other. The pump 15 is then driven.

As a result, the cleaning solution filling the inner space of the cartridge 80 and the solution circulation pipe 14 is discharged through the solution discharge pipe 16.

In this manner, radiation data of a radioactive labeling substance and a fluorescence data of a fluorescent substance such as a fluorescent dye are recorded in a number of the absorptive regions 4 formed in the substrate 2 of the biochemical analysis unit 1.

Similarly to the previous embodiment, the fluorescence data recorded in a number of the absorptive regions 4 of the biochemical analysis unit 1 are read by the scanner shown in FIGS. 9 to 16 and biochemical analysis data are produced.

On the other hand, similarly to the previous embodiment, radiation data recorded in a number of the absorptive regions 4 of the biochemical analysis unit 1 are transferred onto a number of the stimulable phosphor layer regions 20 of the stimulable phosphor sheet 17 shown in FIG. 8 and read by the scanner shown in FIGS. 9 to 16, thereby producing biochemical analysis data.

To the contrary, in order to record chemiluminescence data in a number of the absorptive regions 4 formed in the substrate 2 of the biochemical analysis unit 1, an antibody solution containing an antibody to the hapten such as digoxigenin labeled with an enzyme which generates chemiluminescence emission when it contacts a chemiluminescent substrate is further prepared and accommodated in the antibody solution tank 12 and the antibody to the hapten such as digoxigenin labeled with an enzyme which generates chemiluminescence emission when it contacts a chemiluminescent substrate is bonded with the hapten such as digoxigenin labeling a substance derived from a living organism selectively hybridized with specific binding substances absorbed in a number of the absorptive regions formed in the substrate 2 of the biochemical analysis unit 1 by the an antigen-antibody reaction.

Specifically, an antibody solution containing an antibody to the hapten such as digoxigenin labeled with an enzyme which generates chemiluminescence emission when it contacts a chemiluminescent substrate is first prepared and accommodated in the antibody solution tank 12.

When the antibody solution has been accommodated in the antibody solution tank 12, the change-over valve 16a provided at the bifurcated portion of the solution circulation pipe 14 and the solution discharge pipe 16 is located at its first position and the change-over valve 10b provided in the hybridization buffer feed pipe 10a, the change-over valve 11b provided in the probe solution feed pipe 11a and the change-over valve 13b provided in the cleaning solution feed pipe 13a are located at their second positions where the atmosphere and the solution circulation pipe 14 communicate with each other.

The change-over valve 12b provided in the antibody solution feed pipe 12a is then located at its first position where the antibody solution feed pipe 12a and the solution circulation pipe 14 communicate with each other and the pump 15 is driven, thereby feeding the antibody solution accommodated in the antibody solution tank 12 into the solution circulation pipe 14 via the antibody solution feed pipe 12a.

Since the solution feed passage 81 branches into the n branch passages 83a, 83b, 83c, . . . , 83n whose number is equal to the number of columns of the absorptive regions 4 of the biochemical analysis unit 1, the antibody solution flows into the n branch passages 83a, 83b, 83c, . . . , 83n from the solution feed passage 81.

When the antibody solution flowing in the branch passages 83a, 83b, 83c, . . . , 83n has reached the folded passages 83aa, 83ab, . . . , 83am; 83ba, 83bb, . . . , 83bm; 83ca, 83cb, . . . , 83cm; . . . , 83na, 83nb, . . . , 83nm formed at corresponding absorptive regions 4 of the biochemical analysis unit 1, the antibody solution turns downward as shown in FIG. 23 and comes into contact with the corresponding absorptive region 4 of the biochemical analysis unit 1. The antibody solution then turns upward and flows toward the merged portion with the solution discharge passage 82 of the cartridge 80.

In this manner, the antibody to the hapten such as digoxigenin labeled with an enzyme which generates chemiluminescence emission when it contacts a chemiluminescent substrate is bonded with the hapten such as digoxigenin labeling a substance derived from a living organism selectively hybridized with specific binding substances absorbed in a number of the absorptive regions formed in the substrate 2 of the biochemical analysis unit 1 by the an antigen-antibody reaction.

The antibody solution is fed from the branch passages 83a, 83b, 83c, . . . , 83n to the solution circulation passage 14 of the apparatus for a receptor-ligand association reaction via the merged portion with the solution discharge passage 82 of the cartridge 80 and recycled into the cartridge 80.

In this manner, when an inner space of the cartridge 80 and the solution circulation pipe 14 has been filled with the antibody solution, the change-over valve 12b provided in the antibody solution feed pipe 12a is located at its third position where communication between the antibody solution tank 12 and the atmosphere, and the solution circulation pipe 14 is shut off and the change-over valve 10b provided in the hybridization buffer feed pipe 10a, the change-over valve 11b provided in the probe solution feed pipe 11a and the change-over valve 13b provided in the cleaning solution feed pipe 13a are located at their third positions.

On the other hand, the pump 15 continues to be driven and as a result, the antibody solution filling the inner space of the cartridge 80 and the solution circulation pipe 14 is circulated through the cartridge 80 and the solution circulation pipe 14, whereby the antibody solution is forcibly moved so as to come into contact with a number of the absorptive regions 4 of the biochemical analysis unit 1 held in the cartridge 80.

In this embodiment, since the antibody solution is forcibly fed to only the individual absorptive regions 4 of the biochemical analysis unit 1 held in the cartridge 80 repeatedly in this manner by the folded passages 83aa, 83ab, . . . , 83am; 83ba, 83bb, . . . , 83bm; 83ca, 83cb, . . . , 83cm; . . . , 83na, 83nb, . . . , 83nm, thereby being brought into contact with the individual absorptive regions 4, it is possible to extremely efficiently bind an antibody for the hapten labeled with an enzyme which generates chemiluminescence emission when it contacts a chemiluminescent substrate with the hapten such as digoxigenin labeling a substance derived from a living organism selectively hybridized with specific binding substances fixed in a number of the absorptive regions 4 formed in the substrate 2 of the biochemical analysis unit 1 by the an antigen-antibody reaction in comparison with the case of moving an antibody solution by convection or diffusion and performing the antigen-antibody reaction.

When a fourth predetermined time period has passed, the pump 15 is stopped and the antigen-antibody reaction is completed.

The change-over valve 12b provided in the antibody solution feed pipe 12a is then located at its second position where the atmosphere and the solution circulation pipe 14 communicate with each other and the change-over valve 16a provided at the bifurcated portion of the solution circulation pipe 14 and the solution discharge pipe 16 is located at its second position where the solution circulation pipe 14 and the solution discharge pipe 16 communicate with each other. The pump 15 is then driven.

As a result, the antibody solution filling the inner space of the cartridge 80 and the solution circulation pipe 14 is discharged through the solution discharge pipe 16.

When the antibody solution filling the inner space of the cartridge 80 and the solution circulation pipe 14 has been discharged through the solution discharge pipe 16, the change-over valve 16a provided at the bifurcated portion of the solution circulation pipe 14 and the solution discharge pipe 16 is located at its first position and the change-over valve 10b provided in the hybridization buffer feed pipe 10a, the change-over valve 11b provided in the probe solution feed pipe 11a and the change-over valve 12b provided in the antibody solution feed pipe 12a are located at their second positions where the atmosphere and the solution circulation pipe 14 communicate with each other.

The change-over valve 13b provided in the cleaning solution feed pipe 13a is then located at its first position where the cleaning solution feed pipe 13a and the solution circulation pipe 14 communicate with each other and the pump 15 is driven, thereby feeding a cleaning solution accommodated in the cleaning solution tank 13 into the solution circulation pipe 14 via the cleaning solution feed pipe 13a.

Since the solution feed passage 81 branches into the n branch passages 83a, 83b, 83c, . . . , 83n whose number is equal to the number of columns of the absorptive regions 4 of the biochemical analysis unit 1, the cleaning solution flows into the n branch passages 83a, 83b, 83c, . . . , 83n from the solution feed passage 81.

When the cleaning solution flowing in the branch passages 83a, 83b, 83c, . . . , 83n has reached the folded passages 83aa, 83ab, . . . , 83am; 83ba, 83bb, . . . , 83bm; 83ca, 83cd, . . . , 83cm; . . . , 83na, 83nb, . . . , 83nm formed at corresponding absorptive regions 4 of the biochemical analysis unit 1, the cleaning solution turns downward as shown in FIG. 23 and comes into contact with the corresponding absorptive region 4 of the biochemical analysis unit 1. The cleaning solution then turns upward and flows toward the merged portion with the solution discharge passage 82 of the cartridge 80.

In this manner, the cleaning operation of a number of the absorptive regions 4 of the biochemical analysis unit 1 is performed.

The cleaning solution is fed from the branch passages 83a, 83b, 83c, . . . , 83n to the solution circulation passage 14 of the apparatus for a receptor-ligand association reaction via the merged portion with the solution discharge passage 82 of the cartridge 80 and recycled into the cartridge 80.

In this manner, when an inner space of the cartridge 80 and the solution circulation pipe 14 has been filled with the cleaning solution, the change-over valve 13b provided in the cleaning solution feed pipe 13a is located at its third position where communication between the cleaning solution tank 13 and the atmosphere, and the solution circulation pipe 14 is shut off and the change-over valve 10b provided in the hybridization buffer feed pipe 10a, the change-over valve 11b provided in the probe solution feed pipe 11a and the change-over valve 12b provided in the antibody solution feed pipe 12a are located at their third positions.

On the other hand, the pump 15 continues to be driven and as a result, the cleaning solution filling the inner space of the cartridge 80 and the solution circulation pipe 14 is circulated through the cartridge 80 and the solution circulation pipe 14, whereby the cleaning solution is forcibly moved so as to come into contact with a number of the absorptive regions 4 of the biochemical analysis unit 1 held in the cartridge 80.

In this embodiment, since the cleaning solution is forcibly fed to only the individual absorptive regions 4 of the biochemical analysis unit 1 held in the cartridge 80 repeatedly in this manner by the folded passages 83aa, 83ab, . . . , 83am; 83ba, 83bb, . . . , 83bm; 83ca, 83cb, . . . , 83cm; . . . , 83na, 83nb, 83nm, thereby being brought into contact with the individual absorptive regions 4, it is possible to extremely efficiently clean a number of the absorptive regions 4 of the biochemical analysis unit 1 with the cleaning solution in comparison with the case of moving a cleaning solution by convection or diffusion and cleaning a number of the absorptive regions 4 of the biochemical analysis unit 1 therewith. Therefore, even in the case where an antibody which should not be bonded with the hapten labeling a substance derived from a living body and selectively hybridized with specific binding substances absorbed in the absorptive regions 4 of the biochemical analysis unit 1 has been bonded with the absorptive regions 4 formed in the substrate 2 of the biochemical analysis unit 1, since it is possible to very efficiently peel off and remove the antibody which should not be bonded with the hapten from a number of the absorptive regions 4 of the biochemical analysis unit 1, the efficiency of cleaning operation can be markedly improved.

When a fifth predetermined time period has passed, the pump 15 is stopped and the cleaning operation is completed.

The change-over valve 13b provided in the cleaning solution feed pipe 13a is then located at its second position where the atmosphere and the solution circulation pipe 14 communicate with each other and the change-over valve 16a provided at the bifurcated portion of the solution circulation pipe 14 and the solution discharge pipe 16 is located at its second position where the solution circulation pipe 14 and the solution discharge pipe 16 communicate with each other. The pump 15 is then driven.

As a result, the cleaning solution filling the inner space of the cartridge 80 and the solution circulation pipe 14 is discharged through the solution discharge pipe 16.

In this manner, chemiluminescence data are recorded in a number of the absorptive regions 4 formed in the substrate 2 of the biochemical analysis unit 1.

Similarly to the previous embodiment, the chemiluminescence data thus recorded in a number of the absorptive regions 4 formed in the substrate 2 of the biochemical analysis unit 1 are transferred onto a number of the stimulable phosphor layer regions 77 of the stimulable phosphor sheet 75 shown in FIG. 17 and read by the scanner shown in FIGS. 18 to 20, thereby producing biochemical analysis data.

According to this embodiment, since the mixed solution of the hybridization buffer and the probe solution is forcibly fed to only the individual absorptive regions 4 of the biochemical analysis unit 1 held in the cartridge 80 repeatedly in this manner by the folded passages 83aa, 83ab, . . . , 83am; 83ba, 83bb, . . . , 83b; 83ca, 83cb, . . . , 83cm; . . . , 83na, 83nb, . . . , 83nm, thereby being brought into contact with the individual absorptive regions 4, it is possible to extremely efficiently hybridize a substance derived from a living organism and contained in the mixed solution of the hybridization buffer and the probe solution with specific binding substances fixed in a number of the absorptive regions 4 of the biochemical analysis unit 1 in comparison with the case of moving a substance derived from a living organism and contained in the mixed solution of the hybridization buffer and the probe solution by convection or diffusion and hybridizing it with specific binding substances fixed in a number of the absorptive regions 4 of the biochemical analysis unit 1.

Furthermore, according to this embodiment, since the antibody solution is forcibly fed to only the individual absorptive regions 4 of the biochemical analysis unit 1 held in the cartridge 80 repeatedly in this manner by the folded passages 83aa, 83ab, . . . , 83am; 83ba, 83bb, . . . , 83bm; 83ca, 83cb, . . . , 83cm; . . . , 83na, 83nb, . . . , 83nm, thereby being brought into contact with the individual absorptive regions 4, it is possible to extremely efficiently bind an antibody for the hapten labeled with an enzyme which generates chemiluminescence emission when it contacts a chemiluminescent substrate with the hapten such as digoxigenin labeling a substance derived from a living organism selectively hybridized with specific binding substances fixed in a number of the absorptive regions 4 formed in the substrate 2 of the biochemical analysis unit 1 by the an antigen-antibody reaction in comparison with the case of moving an antibody solution by convection or diffusion and performing the antigen-antibody reaction.

Moreover, according to this embodiment, since the cleaning solution is forcibly fed to only the individual absorptive regions 4 of the biochemical analysis unit 1 held in the cartridge 80 repeatedly in this manner by the folded passages 83aa, 83ab, . . . , 83am; 83ba, 83bb, . . . , 83bm; 83ca, 83cb, . . . , 83cm; . . . , 83na, 83nb, . . . , 83nm, thereby being brought into contact with the individual absorptive regions 4, it is possible to extremely efficiently clean a number of the absorptive regions 4 of the biochemical analysis unit 1 with the cleaning solution in comparison with the case of moving a cleaning solution by convection or diffusion and cleaning a number of the absorptive regions 4 of the biochemical analysis unit 1 therewith. Therefore, even in the case where a substance derived from a living organism which should not be hybridized with specific binding substances fixed in the absorptive regions 4 formed in the substrate 2 of the biochemical analysis unit 1 has been bonded with the absorptive regions 4 during the process of hybridization, since it is possible to very efficiently peel off and remove the substance derived from a living organism which should not be hybridized with specific binding substances fixed in the absorptive regions 4 from a number of the absorptive regions 4 of the biochemical analysis unit 1, it is possible to bind a substance derived from a living organism which is to be hybridized with specific binding substances fixed in a number of the absorptive regions 4 of the biochemical analysis unit 1 therewith in a desired manner.

Further, according to this embodiment, since the cleaning solution is forcibly fed to only the individual absorptive regions 4 of the biochemical analysis unit 1 held in the cartridge 80 repeatedly in this manner by the folded passages 83aa, 83ab, . . . , 83am; 83ba, 83bb, . . . , 83bm; 83ca, 83cb, . . . , 83cm; . . . , 83na, 83nb, . . . , 83nm, thereby being brought into contact with the individual absorptive regions 4, it is possible to extremely efficiently clean a number of the absorptive regions 4 of the biochemical analysis unit 1 with the cleaning solution in comparison with the case of moving a cleaning solution by convection or diffusion and cleaning a number of the absorptive regions 4 of the biochemical analysis unit 1 therewith. Therefore, even in the case where an antibody which should not be bonded with the hapten labeling a substance derived from a living body and selectively hybridized with specific binding substances absorbed in the absorptive regions 4 of the biochemical analysis unit 1 has been bonded with the absorptive regions 4 formed in the substrate 2 of the biochemical analysis unit 1, since it is possible to very efficiently peel off and remove the antibody which should not be bonded with the hapten from a number of the absorptive regions 4 of the biochemical analysis unit 1, the efficiency of cleaning operation can be markedly improved.

Furthermore, according to this embodiment, since the mixed solution of the hybridization buffer and the probe solution is forcibly fed to only the absorptive regions 4 of the biochemical analysis unit 1, it is possible to reliably prevent a substance derived from a living organism from adhering to portions of the substrate 2 of the biochemical analysis unit 1 other than the absorptive regions 4. Therefore, since it is sufficient to feed a cleaning solution to only the absorptive regions 4 of the biochemical analysis unit 1, thereby cleaning them, the efficiency of the cleaning operation can be improved.

Moreover, according to this embodiment, since the antibody solution is forcibly fed to only the absorptive regions 4 of the biochemical analysis unit 1, it is possible to reliably prevent an antibody from adhering to portions of the substrate 2 of the biochemical analysis unit 1 other than the absorptive regions 4. Therefore, since it is sufficient to feed a cleaning solution to only the absorptive regions 4 of the biochemical analysis unit 1, thereby cleaning them, the efficiency of the cleaning operation can be improved.

Further, according to this embodiment, since each of the folded passages 83aa, 83ab, . . . , 83am; 83ba, 83bb, . . . , 83bm; 83ca, 83cb, . . . , 83cm; . . . , 83na, 83nb, . . . , 83nm is formed so that the length thereof is equal to or shorter than 0.5 mm, preferably, 0.1 mm, the reaction can be facilitated in accordance with the principle of a micro-reactor, the efficiency of the hybridization, the efficiency of the antigen-antibody reaction and the efficiency of the cleaning operation can be markedly improved.

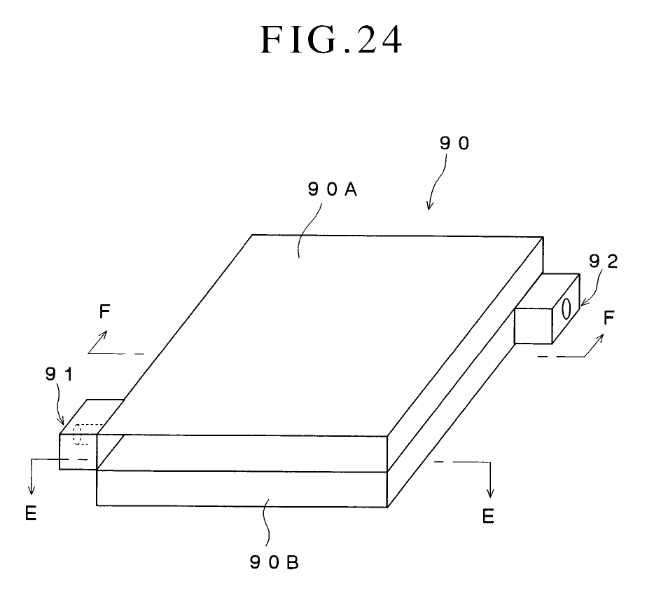

FIG. 24 is a schematic perspective view showing a cartridge for a biochemical analysis unit which is a further preferred embodiment of the present invention.

As shown in FIG. 24, the cartridge 90 for a biochemical analysis unit according to this embodiment includes an upper half portion 90A and a lower half portion 90B and the biochemical analysis unit 1 is held between the upper half portion 90A and the lower half portion 90B.

As shown in FIG. 24, a solution feed passage 91 is formed for feeding a solution into the cartridge 90 on the side surface of the upper half portion 90A of the cartridge 90 in the vicinity of one corner thereof and a solution discharge passage 92 is formed for discharging a solution from the cartridge 90 on the side surface of the upper half portion 90A of the cartridge 90 in the vicinity of the corner thereof diagonally positioned to the above mentioned corner.

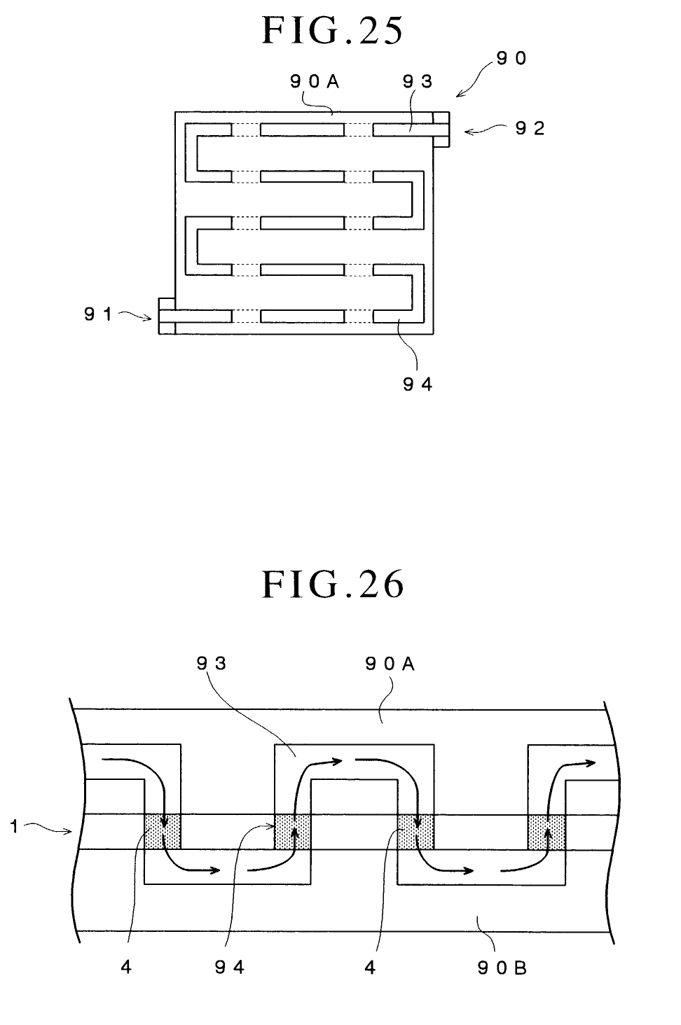

FIG. 25 is a schematic cross sectional view taken along a line E-E in FIG. 24.

As shown in FIG. 25, the cartridge 90 is formed with a solution passage 93 connected to the solution feed passage 91 at the upstream end portion thereof, connected to the solution discharge passage 92 at the downstream portion thereof and extending along the column of a number of the absorptive regions 4 of the biochemical analysis unit 1 held in the cartridge 90.

FIG. 26 is a schematic cross sectional view taken along a line F-F in FIG. 24.

As shown in FIG. 26, the solution passage 93 includes through passages 94 crossing a number of the absorptive regions 4 of the biochemical analysis unit 1 held in the cartridge 90 in a direction substantially perpendicular to the surface of the substrate 2 of the biochemical analysis unit 1, the number of which is equal to that of the absorptive regions 4 so that a solution can pass through the individual absorptive regions 4 of the biochemical analysis unit 1 via the through passages 94 and flow in the solution passage 93 from the solution feed passage 91 toward the solution discharge passage 92.

In this embodiment, a portion of the solution passage 93 corresponding to each column of the absorptive regions 4 is formed in the cartridge 90 so that each of odd numbered through passages 94 from the upstream with respect to the solution flowing direction can feed a solution so as to pass through the corresponding absorptive region 4 downward and that each of even numbered through passages from the upstream with respect to the solution flowing direction can feed a solution so as to pass through the corresponding absorptive region 4 upward and each of the through passages has the same size as that of each of the absorptive regions 4 formed in the substrate 2 of the biochemical analysis unit 1, namely, has a size of about 0.01 mm².

When a substance derived from a living organism and labeled with a labeling substance is to be selectively hybridized with specific binding substances fixed in a number of the absorptive regions 4 of the biochemical analysis unit 1, the thus constituted cartridge 90 for a biochemical analysis unit holding the biochemical analysis unit 1 therein is first set on the support base 7C of the apparatus for a receptor-ligand association reaction. One end portion of the solution circulating pipe 14 of the apparatus for a receptor-ligand association reaction is then connected to the solution feed passage 91 of the cartridge 90 and the other end portion of the solution circulating pipe 14 is connected to the solution discharge passage 92 of the cartridge 90.

When hybridization is to be performed, a hybridization buffer is first prepared and accommodated in the hybridization buffer tank 10.

Then, the change-over valve 11b provided in the probe solution feed pipe 11a, the change-over valve 12b provided in the antibody solution feed pipe 12a and the change-over valve 13b provided in the cleaning solution feed pipe 13a are located at their second positions where the atmosphere and the solution circulation pipe 14 communicate with each other and the change-over valve 16a provided at the bifurcated portion of the solution circulation pipe 14 and the solution discharge pipe 16 is located at its first position.

When the change-over valve 10b provided in the hybridization buffer feed pipe 10a has been located at its first position where the hybridization buffer feed pipe 10a and the solution circulation pipe 14 communicate with each other, the pump 15 is driven.

As a result, a hybridization buffer accommodated in the hybridization buffer tank 10 is fed into the solution feed passage 91 formed in the cartridge 90 via the hybridization buffer feed pipe 10a and the solution circulation pipe 14.

The hybridization buffer fed into the solution feed passage 91 flows into the solution passage 93 and flows through the solution passage 93 toward the solution discharge passage 92 in such a manner that when the hybridization buffer reaches a through passage 94 formed at an even numbered absorptive region 4 of the biochemical analysis unit 1 held in the cartridge 90, the hybridization buffer turns its direction downward and passes through the absorptive region 4 as shown in FIG. 26 and that when the hybridization buffer reaches a through passage 94 formed at an odd numbered absorptive region 4 of the biochemical analysis unit 1 held in the cartridge 90, the hybridization buffer turns its direction upward and passes through the absorptive region 4 as shown in FIG. 26.

In this manner, pre-hybridization is performed.

The hybridization buffer is fed from the solution passage 93 to the solution circulation passage 14 of the apparatus for a receptor-ligand association reaction via the solution discharge passage 92 of the cartridge 90 and recycled into the cartridge 90.

In this manner, when an inner space of the cartridge 90 and the solution circulation pipe 14 has been filled with the hybridization buffer, the change-over valve 10b provided in the hybridization buffer feed pipe 10a is located at its third position where communication between the hybridization buffer tank 10 and the atmosphere, and the solution circulation pipe 14 is shut off and the change-over valve 11b provided in the probe solution feed pipe 11a, the change-over valve 12b provided in the antibody solution feed pipe 12a and the change-over valve 13b provided in the cleaning solution feed pipe 13a are located at their third positions.

On the other hand, the pump 15 continues to be driven and as a result, the hybridization buffer filling the inner space of the cartridge 90 and the solution circulation pipe 14 is circulated through the cartridge 90 and the solution circulation pipe 14, whereby the hybridization buffer is forcibly moved in the solution passage 93 so as to sequentially pass through a number of the absorptive regions 4 of the biochemical analysis unit 1 held in the cartridge 90.

When a first predetermined time period has passed, the pump 15 is stopped and pre-hybridization is completed.

Then, a probe solution is prepared and accommodated in the probe solution chip 11.

Similarly to the previous embodiments, in this embodiment, a probe solution containing a substance derived from a living organism and labeled with a radioactive labeling substance, a substance derived from a living organism and labeled with a fluorescent substance such as a fluorescent dye and a substance derived from a living organism and labeled with hapten such as digoxigenin is prepared and accommodated in the probe solution chip 11.

When the probe solution has been accommodated in the probe solution chip 11, the change-over valve 10b provided in the hybridization buffer feed pipe 10a, the change-over valve 12b provided in the antibody solution feed pipe 12a and the change-over valve 13b provided in the cleaning solution feed pipe 13a are located at their second positions where the atmosphere and the solution circulation pipe 14 communicate with each other and the change-over valve 16a provided at the bifurcated portion of the solution circulation pipe 14 and the solution discharge pipe 16 is located at its first position.

Then, the change-over valve 11b provided in the probe solution feed pipe 11a is located at its first position where the probe solution feed pipe 11a and the solution circulation pipe 14 communicate with each other and the pump 15 is driven.

As a result, a probe solution accommodated in the probe solution chip 11 is fed into the solution circulation pipe 14 via the probe solution feed pipe 11a and mixed with the hybridization buffer filling the inner space of the cartridge 90 and the solution circulation pipe 14.

When a predetermined amount of the probe solution has been fed from the probe solution chip 11, the change-over valve 11b provided in the probe solution feed pipe 11a is located at its third position where communication between the probe solution tank 11 and the atmosphere, and the solution circulation pipe 14 is shut off and the change-over valve 10b provided in the hybridization buffer feed pipe 10a, the change-over valve 12b provided in the antibody solution feed pipe 12a and the change-over valve 13b provided in the cleaning solution feed pipe 13a are located at their third positions.

On the other hand, the pump 15 continues to be driven and therefore, the mixed solution produced by mixing the probe solution with the hybridization buffer filling the inner space of the cartridge 90 and the solution circulation pipe 14 is fed into the solution feed passage 91 formed in the cartridge 90 from the solution circulation pipe 14.

The mixed solution of the hybridization buffer and the probe solution fed into the solution feed passage 91 flows into the solution passage 93 and flows through the solution passage 93 toward the solution discharge passage 92 in such a manner that when the mixed solution of the hybridization buffer and the probe solution reaches a through passage 94 formed at an even numbered absorptive region 4 of the biochemical analysis unit 1 held in the cartridge 90, the mixed solution of the hybridization buffer and the probe solution turns its direction downward and passes through the absorptive region 4 as shown in FIG. 26 and that when the mixed solution of the hybridization buffer and the probe solution reaches a through passage 94 formed at an odd numbered absorptive region 4 of the biochemical analysis unit 1 held in the cartridge 90, the mixed solution of the hybridization buffer and the probe solution turns its direction upward and passes through the absorptive region 4 as shown in FIG. 26.

As a result, a substance derived from a living organism and contained in the mixed solution of the hybridization buffer and the probe solution selectively hybridizes with specific binding substances absorbed in a number of the absorptive regions 4 formed in the substrate 2 of the biochemical analysis unit 1 held in the cartridge 90.

The mixed solution of the hybridization buffer and the probe solution is fed from the solution passage 93 to the solution circulation passage 14 of the apparatus for a receptor-ligand association reaction via the solution discharge passage 92 of the cartridge 90 and recycled into the cartridge 90.

In this embodiment, since the mixed solution of the hybridization buffer and the probe solution is circulated through the cartridge 90 and the solution circulation pipe 14, whereby the mixed solution of the hybridization buffer and the probe solution is forcibly moved in the solution passage 93 so as to sequentially pass through a number of the absorptive regions 4 of the biochemical analysis unit 1 held in the cartridge 90, it is possible to extremely efficiently hybridize a substance derived from a living organism and contained in the mixed solution of the hybridization buffer and the probe solution with specific binding substances fixed in a number of the absorptive regions 4 of the biochemical analysis unit 1 in comparison with the case of moving a substance derived from a living organism and contained in the mixed solution of the hybridization buffer and the probe solution by convection or diffusion and hybridizing it with specific binding substances fixed in a number of the absorptive regions 4 of the biochemical analysis unit 1.

When a second predetermined time period has passed, the pump 15 is stopped and hybridization is completed.

The change-over valve 11b provided in the probe solution feed pipe 11a is then located at its second position where the atmosphere and the solution circulation pipe 14 communicate with each other and the change-over valve 16a provided at the bifurcated portion of the solution circulation pipe 14 and the solution discharge pipe 16 is located at its second position where the solution circulation pipe 14 and the solution discharge pipe 16 communicate with each other. The pump 15 is then driven.

As a result, the mixed solution of the hybridization buffer and the probe solution filling the inner space of the cartridge 90 and the solution circulation pipe 14 is discharged through the solution discharge pipe 16.

When the mixed solution of the hybridization buffer and the probe solution filling the inner space of the cartridge 90 and the solution circulation pipe 14 has been discharged through the solution discharge pipe 16, the change-over valve 16a provided at the bifurcated portion of the solution circulation pipe 14 and the solution discharge pipe 16 is located at its first position and the change-over valve 10b provided in the hybridization buffer feed pipe 10a, the change-over valve 11b provided in the probe solution feed pipe 11a and the change-over valve 12b provided in the antibody solution feed pipe 12a are located at their second positions where the atmosphere and the solution circulation pipe 14 communicate with each other.

The change-over valve 13b provided in the cleaning solution feed pipe 13a is then located at its first position where the cleaning solution feed pipe 13a and the solution circulation pipe 14 communicate with each other and the pump 15 is driven, thereby feeding a cleaning solution accommodated in the cleaning solution tank 13 into the solution circulation pipe 14 via the cleaning solution feed pipe 13a.

The cleaning solution fed into the solution feed passage 91 flows into the solution passage 93 and flows through the solution passage 93 toward the solution discharge passage 92 in such a manner that when the cleaning solution reaches a through passage 94 formed at an even numbered absorptive region 4 of the biochemical analysis unit 1 held in the cartridge 90, the cleaning solution turns its direction downward and passes through the absorptive region 4 as shown in FIG. 26 and that when the cleaning solution reaches a through passage 94 formed at an odd numbered absorptive region 4 of the biochemical analysis unit 1 held in the cartridge 90, the cleaning solution turns its direction upward and passes through the absorptive region 4 as shown in FIG. 26.

In this manner, the cleaning operation of a number of the absorptive regions 4 of the biochemical analysis unit 1 is performed.

The cleaning solution is fed from the solution passage 93 to the solution circulation passage 14 of the apparatus for a receptor-ligand association reaction via the solution discharge passage 92 of the cartridge 90 and recycled into the cartridge 90.

In this embodiment, since the cleaning solution is circulated through the cartridge 90 and the solution circulation pipe 14, whereby the cleaning solution is forcibly moved in the solution passage 93 so as to sequentially pass through a number of the absorptive regions 4 of the biochemical analysis unit 1 held in the cartridge 90, it is possible to extremely efficiently clean a number of the absorptive regions 4 of the biochemical analysis unit 1 with the cleaning solution in comparison with the case of moving a cleaning solution by convection or diffusion and cleaning a number of the absorptive regions 4 of the biochemical analysis unit 1 therewith. Therefore, even in the case where a substance derived from a living organism which should not be hybridized with specific binding substances fixed in the absorptive regions 4 formed in the substrate 2 of the biochemical analysis unit 1 has been bonded with the absorptive regions 4 during the process of hybridization, since it is possible to very efficiently peel off and remove the substance derived from a living organism which should not be hybridized with specific binding substances fixed in the absorptive regions 4 from a number of the absorptive regions 4 of the biochemical analysis unit 1, it is possible to bind a substance derived from a living organism which is to be hybridized with specific binding substances fixed in a number of the absorptive regions 4 of the biochemical analysis unit 1 therewith in a desired manner.

When a third predetermined time period has passed, the pump 15 is stopped and the cleaning operation is completed.

The change-over valve 13b provided in the cleaning solution feed pipe 13a is then located at its second position where the atmosphere and the solution circulation pipe 14 communicate with each other and the change-over valve 16a provided at the bifurcated portion of the solution circulation pipe 14 and the solution discharge pipe 16 is located at its second position where the solution circulation pipe 14 and the solution discharge pipe 16 communicate with each other. The pump 15 is then driven.

As a result, the cleaning solution filling the inner space of the cartridge 90 and the solution circulation pipe 14 is discharged through the solution discharge pipe 16.

In this manner, radiation data of a radioactive labeling substance and a fluorescence data of a fluorescent substance such as a fluorescent dye are recorded in a number of the absorptive regions 4 formed in the substrate 2 of the biochemical analysis unit 1.

Similarly to the previous embodiments, the fluorescence data recorded in a number of the absorptive regions 4 of the biochemical analysis unit 1 are read by the scanner shown in FIGS. 9 to 16 and biochemical analysis data are produced.

On the other hand, similarly to the previous embodiments, radiation data recorded in a number of the absorptive regions 4 of the biochemical analysis unit 1 are transferred onto a number of the stimulable phosphor layer regions 20 of the stimulable phosphor sheet 17 shown in FIG. 7 and read by the scanner shown in FIGS. 9 to 16, thereby producing biochemical analysis data.

To the contrary, in order to record chemiluminescence data in a number of the absorptive regions 4 formed in the substrate 2 of the biochemical analysis unit 1, an antibody solution containing an antibody to the hapten such as digoxigenin labeled with an enzyme which generates chemiluminescence emission when it contacts a chemiluminescent substrate is further prepared and accommodated in the antibody solution tank 12 and the antibody to the hapten such as digoxigenin labeled with an enzyme which generates chemiluminescence emission when it contacts a chemiluminescent substrate is bonded with the hapten such as digoxigenin labeling a substance derived from a living organism selectively hybridized with specific binding substances absorbed in a number of the absorptive regions formed in the substrate 2 of the biochemical analysis unit 1 by the an antigen-antibody reaction.

Specifically, an antibody solution containing an antibody to the hapten such as digoxigenin labeled with an enzyme which generates chemiluminescence emission when it contacts a chemiluminescent substrate is first prepared and accommodated in the antibody solution tank 12.

When the antibody solution has been accommodated in the antibody solution tank 12, the change-over valve 16a provided at the bifurcated portion of the solution circulation pipe 14 and the solution discharge pipe 16 is located at its first position and the change-over valve 10b provided in the hybridization buffer feed pipe 10a, the change-over valve 11b provided in the probe solution feed pipe 11a and the change-over valve 13b provided in the cleaning solution feed pipe 13a are located at their second positions where the atmosphere and the solution circulation pipe 14 communicate with each other.

The change-over valve 12b provided in the antibody solution feed pipe 12a is then located at its first position where the antibody solution feed pipe 12a and the solution circulation pipe 14 communicate with each other and the pump 15 is driven, thereby feeding the antibody solution accommodated in the antibody solution tank 12 into the solution circulation pipe 14 via the antibody solution feed pipe 12a.

The antibody solution fed into the solution feed passage 91 flows into the solution passage 93 and flows through the solution passage 93 toward the solution discharge passage 92 in such a manner that when the antibody solution reaches a through passage 94 formed at an even numbered absorptive region 4 of the biochemical analysis unit 1 held in the cartridge 90, the antibody solution turns its direction downward and passes through the absorptive region 4 as shown in FIG. 26 and that when the antibody solution reaches a through passage 94 formed at an odd numbered absorptive region 4 of the biochemical analysis unit 1 held in the cartridge 90, the antibody solution turns its direction upward and passes through the absorptive region 4 as shown in FIG. 26.

In this manner, the antibody to the hapten such as digoxigenin labeled with an enzyme which generates chemiluminescence emission when it contacts a chemiluminescent substrate is bonded with the hapten such as digoxigenin labeling a substance derived from a living organism selectively hybridized with specific binding substances absorbed in a number of the absorptive regions formed in the substrate 2 of the biochemical analysis unit 1 by the an antigen-antibody reaction.

The antibody solution is fed from the solution passage 93 to the solution circulation passage 14 of the apparatus for a receptor-ligand association reaction via the solution discharge passage 92 of the cartridge 90 and recycled into the cartridge 90.

In this embodiment, since the antibody solution is circulated through the cartridge 90 and the solution circulation pipe 14, whereby the antibody solution is forcibly moved in the solution passage 93 so as to sequentially pass through a number of the absorptive regions 4 of the biochemical analysis unit 1 held in the cartridge 90, it is possible to extremely efficiently bind an antibody for the hapten labeled with an enzyme which generates chemiluminescence emission when it contacts a chemiluminescent substrate with the hapten such as digoxigenin labeling a substance derived from a living organism selectively hybridized with specific binding substances fixed in a number of the absorptive regions 4 formed in the substrate 2 of the biochemical analysis unit 1 by the an antigen-antibody reaction in comparison with the case of moving an antibody solution by convection or diffusion and performing the antigen-antibody reaction.

When a fourth predetermined time period has passed, the pump 15 is stopped and the antigen-antibody reaction is completed.

The change-over valve 12b provided in the antibody solution feed pipe 12a is then located at its second position where the atmosphere and the solution circulation pipe 14 communicate with each other and the change-over valve 16a provided at the bifurcated portion of the solution circulation pipe 14 and the solution discharge pipe 16 is located at its second position where the solution circulation pipe 14 and the solution discharge pipe 16 communicate with each other. The pump 15 is then driven.

As a result, the antibody solution filling the inner space of the cartridge 90 and the solution circulation pipe 14 is discharged through the solution discharge pipe 16.

When the antibody solution filling the inner space of the cartridge 90 and the solution circulation pipe 14 has been discharged through the solution discharge pipe 16, the change-over valve 16a provided at the bifurcated portion of the solution circulation pipe 14 and the solution discharge pipe 16 is located at its first position and the change-over valve 10b provided in the hybridization buffer feed pipe 10a, the change-over valve 11b provided in the probe solution feed pipe 11a and the change-over valve 12b provided in the antibody solution feed pipe 12a are located at their second positions where the atmosphere and the solution circulation pipe 14 communicate with each other.

The change-over valve 13b provided in the cleaning solution feed pipe 13a is then located at its first position where the cleaning solution feed pipe 13a and the solution circulation pipe 14 communicate with each other and the pump 15 is driven, thereby feeding a cleaning solution accommodated in the cleaning solution tank 13 into the solution circulation pipe 14 via the cleaning solution feed pipe 13a.

The cleaning solution fed into the solution feed passage 91 flows into the solution passage 93 and flows through the solution passage 93 toward the solution discharge passage 92 in such a manner that when the cleaning solution reaches a through passage 94 formed at an even numbered absorptive region 4 of the biochemical analysis unit 1 held in the cartridge 90, the cleaning solution turns its direction downward and passes through the absorptive region 4 as shown in FIG. 26 and that when the cleaning solution reaches a through passage 94 formed at an odd numbered absorptive region 4 of the biochemical analysis unit 1 held in the cartridge 90, the cleaning solution turns its direction upward and passes through the absorptive region 4 as shown in FIG. 26.

In this manner, the cleaning operation of a number of the absorptive regions 4 of the biochemical analysis unit 1 is performed.

The cleaning solution is fed from the solution passage 93 to the solution circulation passage 14 of the apparatus for a receptor-ligand association reaction via the solution discharge passage 92 of the cartridge 90 and recycled into the cartridge 90.

In this embodiment, since the cleaning solution is circulated through the cartridge 90 and the solution circulation pipe 14, whereby the cleaning solution is forcibly moved in the solution passage 93 so as to sequentially pass through a number of the absorptive regions 4 of the biochemical analysis unit 1 held in the cartridge 90, it is possible to extremely efficiently clean a number of the absorptive regions 4 of the biochemical analysis unit 1 with the cleaning solution in comparison with the case of moving a cleaning solution by convection or diffusion and cleaning a number of the absorptive regions 4 of the biochemical analysis unit 1 therewith. Therefore, even in the case where an antibody which should not be bonded with the hapten labeling a substance derived from a living body and selectively hybridized with specific binding substances absorbed in the absorptive regions 4 of the biochemical analysis unit 1 has been bonded with the absorptive regions 4 formed in the substrate 2 of the biochemical analysis unit 1, since it is possible to very efficiently peel off and remove the antibody which should not be bonded with the hapten from a number of the absorptive regions 4 of the biochemical analysis unit 1, the efficiency of cleaning operation can be markedly improved.

When a fifth predetermined time period has passed, the pump 15 is stopped and the cleaning operation is completed.

The change-over valve 13b provided in the cleaning solution feed pipe 13a is then located at its second position where the atmosphere and the solution circulation pipe 14 communicate with each other and the change-over valve 16a provided at the bifurcated portion of the solution circulation pipe 14 and the solution discharge pipe 16 is located at its second position where the solution circulation pipe 14 and the solution discharge pipe 16 communicate with each other. The pump 15 is then driven.

As a result, the cleaning solution filling the inner space of the cartridge 90 and the solution circulation pipe 14 is discharged through the solution discharge pipe 16.

In this manner, chemiluminescence data are recorded in a number of the absorptive regions 4 formed in the substrate 2 of the biochemical analysis unit 1.

Similarly to the previous embodiments, the chemiluminescence data thus recorded in a number of the absorptive regions 4 formed in the substrate 2 of the biochemical analysis unit 1 are transferred onto a number of the stimulable phosphor layer regions 77 of the stimulable phosphor sheet 75 shown in FIG. 17 and read by the scanner shown in FIGS. 18 to 20, thereby producing biochemical analysis data.

According to this embodiment, since the mixed solution of the hybridization buffer and the probe solution is circulated through the cartridge 90 and the solution circulation pipe 14, whereby the mixed solution of the hybridization buffer and the probe solution is forcibly moved in the solution passage 93 so as to sequentially pass through a number of the absorptive regions 4 of the biochemical analysis unit 1 held in the cartridge 90, it is possible to extremely efficiently hybridize a substance derived from a living organism and contained in the mixed solution of the hybridization buffer and the probe solution with specific binding substances fixed in a number of the absorptive regions 4 of the biochemical analysis unit 1 in comparison with the case of moving a substance derived from a living organism and contained in the mixed solution of the hybridization buffer and the probe solution by convection or diffusion and hybridizing it with specific binding substances fixed in a number of the absorptive regions 4 of the biochemical analysis unit 1.

Further, according to this embodiment, since the antibody solution is circulated through the cartridge 90 and the solution circulation pipe 14, whereby the antibody solution is forcibly moved in the solution passage 93 so as to sequentially pass through a number of the absorptive regions 4 of the biochemical analysis unit 1 held in the cartridge 90, it is possible to extremely efficiently bind an antibody for the hapten labeled with an enzyme which generates chemiluminescence emission when it contacts a chemiluminescent substrate with the hapten such as digoxigenin labeling a substance derived from a living organism selectively hybridized with specific binding substances fixed in a number of the absorptive regions 4 formed in the substrate 2 of the biochemical analysis unit 1 by the an antigen-antibody reaction in comparison with the case of moving an antibody solution by convection or diffusion and performing the antigen-antibody reaction.

Furthermore, according to this embodiment, since the cleaning solution is circulated through the cartridge 90 and the solution circulation pipe 14, whereby the cleaning solution is forcibly moved in the solution passage 93 so as to sequentially pass through a number of the absorptive regions 4 of the biochemical analysis unit 1 held in the cartridge 90, it is possible to extremely efficiently clean a number of the absorptive regions 4 of the biochemical analysis unit 1 with the cleaning solution in comparison with the case of moving a cleaning solution by convection or diffusion and cleaning a number of the absorptive regions 4 of the biochemical analysis unit 1 therewith. Therefore, even in the case where a substance derived from a living organism which should not be hybridized with specific binding substances fixed in the absorptive regions 4 formed in the substrate 2 of the biochemical analysis unit 1 has been bonded with the absorptive regions 4 during the process of hybridization, since it is possible to very efficiently peel off and remove the substance derived from a living organism which should not be hybridized with specific binding substances fixed in the absorptive regions 4 from a number of the absorptive regions 4 of the biochemical analysis unit 1, it is possible to bind a substance derived from a living organism which is to be hybridized with specific binding substances fixed in a number of the absorptive regions 4 of the biochemical analysis unit 1 therewith in a desired manner.

Moreover, according to this embodiment, since the cleaning solution is circulated through the cartridge 90 and the solution circulation pipe 14, whereby the cleaning solution is forcibly moved in the solution passage 93 so as to sequentially pass through a number of the absorptive regions 4 of the biochemical analysis unit 1 held in the cartridge 90, it is possible to extremely efficiently clean a number of the absorptive regions 4 of the biochemical analysis unit 1 with the cleaning solution in comparison with the case of moving a cleaning solution by convection or diffusion and cleaning a number of the absorptive regions 4 of the biochemical analysis unit 1 therewith. Therefore, even in the case where an antibody which should not be bonded with the hapten labeling a substance derived from a living body and selectively hybridized with specific binding substances absorbed in the absorptive regions 4 of the biochemical analysis unit 1 has been bonded with the absorptive regions 4 formed in the substrate 2 of the biochemical analysis unit 1, since it is possible to very efficiently peel off and remove the antibody which should not be bonded with the hapten from a number of the absorptive regions 4 of the biochemical analysis unit 1, the efficiency of cleaning operation can be markedly improved.

Further, according to this embodiment, since the mixed solution of the hybridization buffer and the probe solution is forcibly fed to only the absorptive regions 4 of the biochemical analysis unit 1, it is possible to reliably prevent a substance derived from a living organism from adhering to portions of the substrate 2 of the biochemical analysis unit 1 other than the absorptive regions 4. Therefore, since it is sufficient to feed a cleaning solution to only the absorptive regions 4 of the biochemical analysis unit 1, thereby cleaning them, the efficiency of the cleaning operation can be improved.

Furthermore, according to this embodiment, since the antibody solution is forcibly fed to only the absorptive regions 4 of the biochemical analysis unit 1, it is possible to reliably prevent an antibody from adhering to portions of the substrate 2 of the biochemical analysis unit 1 other than the absorptive regions 4. Therefore, since it is sufficient to feed a cleaning solution to only the absorptive regions 4 of the biochemical analysis unit 1, thereby cleaning them, the efficiency of the cleaning operation can be improved.

Moreover, according to this embodiment, since each of the through passages 94 is formed so as to have the same size as that of each of the absorptive regions 4 of the biochemical analysis unit 1 and each of the absorptive regions 4 formed in the substrate 2 of the biochemical analysis unit 1 has a size of about 0.01 mm², the reaction can be caused within a micro-area of about 0.01 mm². Therefore, since the reaction can be facilitated in accordance with the principle of a micro-reactor, the efficiency of the hybridization, the efficiency of the antigen-antibody reaction and the efficiency of the cleaning operation can be markedly improved.

The present invention has thus been shown and described with reference to specific embodiments. However, it should be noted that the present invention is in no way limited to the details of the described arrangements but changes and modifications may be made without departing from the scope of the appended claims.

For example, in the above described embodiments, radiation data, fluorescence data and chemiluminescence data are selectively recorded in a number of the absorptive regions 4 of the biochemical analysis unit 1 by selectively hybridizing a substance derived from a living organism and labeled with a radioactive labeling substance and a fluorescent substance with specific labeling substances fixed in a number of the absorptive regions 4 of the biochemical analysis unit 1, selectively hybridizing a substance derived from a living organism and labeled with hapten such as digoxigenin with specific labeling substances fixed in a number of the absorptive regions 4 of the biochemical analysis unit 1 and further binding an antibody for the hapten labeled with an enzyme which generates chemiluminescence emission when it contacts a chemiluminescent substrate with the hapten labeling a substance derived from a living organism selectively hybridized with the specific binding substances by an antigen-antibody reaction. However, the application of the present invention is not limited to such reaction and the present invention can be applied to various kinds of a receptor-ligand association reactions.

Further, in the above described embodiments, chemiluminescence data are selectively recorded in a number of the absorptive regions 4 of the biochemical analysis unit 1 by selectively hybridizing a substance derived from a living organism and labeled with hapten such as digoxigenin with specific labeling substances fixed in a number of the absorptive regions 4 of the biochemical analysis unit 1 and further binding an antibody for the hapten labeled with an enzyme which generates chemiluminescence emission when it contacts a chemiluminescent substrate with the hapten labeling a substance derived from a living organism and selectively hybridized with the specific binding substances fixed in number of the absorptive regions 4 of the biochemical analysis unit 1 by an antigen-antibody reaction. However, chemiluminescence data may be selectively recorded in a number of the absorptive regions 4 of the biochemical analysis unit 1 by selectively hybridizing a substance derived from a living body and labeled with a labeling substance which generates chemiluminescence emission when it contacts a chemiluminescent substrate with specific binding substances fixed in a number of the absorptive regions 4 of the biochemical analysis unit 1.

Furthermore, in the above described embodiments, fluorescence data are selectively recorded in a number of the absorptive regions 4 of the biochemical analysis unit 1 by selectively hybridizing a substance derived from a living organism and labeled with a fluorescent substance with specific labeling substances fixed in a number of the absorptive regions 4 of the biochemical analysis unit 1. However, fluorescence data may be selectively recorded in a number of the absorptive regions 4 of the biochemical analysis unit 1 by selectively hybridizing a substance derived from a living organism and labeled with hapten such as digoxigenin with specific labeling substances fixed in a number of the absorptive regions 4 of the biochemical analysis unit 1 and further binding an antibody for the hapten labeled with an enzyme which generates a fluorescence substance when it contacts a fluorescent substrate with the hapten labeling a substance derived from a living organism and selectively hybridized with the specific binding substances fixed in number of the absorptive regions 4 of the biochemical analysis unit 1 by an antigen-antibody reaction.

Further, in the above described embodiments, the probe solution containing a substance derived from a living organism and labeled with a radioactive labeling substance, a fluorescent substance and hapten such as digoxigenin is prepared and the substance derived from a living organism and labeled with a radioactive labeling substance, a fluorescent substance and hapten such as digoxigenin is selectively hybridized with specific binding substances fixed in number of the absorptive regions 4 of the biochemical analysis unit 1. However, it is not absolutely necessary for the probe solution to contain a substance derived from a living organism and labeled with a radioactive labeling substance, a fluorescent substance and hapten such as digoxigenin and it is sufficient for probe solution to contain a substance derived from a living organism and labeled with at least one of a radioactive labeling substance, a fluorescent substance and hapten such as digoxigenin.

Moreover, in the above described embodiments, as specific binding substances, cDNAs each of which has a known base sequence and is different from the others are used. However, specific binding substances usable in the present invention are not limited to cDNAs but all specific binding substances capable of specifically binding with a substance derived from a living organism such as a cell, virus, hormone, tumor marker, enzyme, antibody, antigen, abzyme, other protein, a nuclear acid, cDNA, DNA, RNA or the like and whose sequence, base length, composition and the like are known, can be employed in the present invention as a specific binding substance.

Further, in the above described embodiments, although the hybridization, the antigen-antibody reaction and the cleaning of a number of the absorptive regions 4 of the biochemical analysis unit 1 are performed by the apparatus for receptor-ligand association reaction, it is possible to perform only the hybridization or the antigen-antibody reaction using the apparatus for receptor-ligand association reaction and perform a number of the absorptive regions 4 of the biochemical analysis unit 1 using a separate cleaning apparatus.

Moreover, in the above described embodiments, the pump 15 of the apparatus for the receptor-ligand association reaction is driven only in one direction and the apparatus for the receptor-ligand association reaction is constituted so as to feed a hybridization buffer, a mixed solution of a hybridization buffer and a probe solution, an antibody solution and a cleaning solution through a number of the absorptive regions 4 of the biochemical analysis unit 1 held in the cartridge 7, 80, 90 by the pump 15 in one direction. However, it is possible to constitute the pump 15 to be driven in both an forward direction and a reverse direction and to constitute the apparatus for the receptor-ligand association reaction so as to feed a hybridization buffer, a mixed solution of a hybridization buffer and a probe solution, an antibody solution and a cleaning solution through a number of the absorptive regions 4 of the biochemical analysis unit 1 held in the cartridge 7, 80, 90 by the pump 15 in both the forward direction and the reverse direction.

Furthermore, in the above described embodiments, although the apparatus for the receptor-ligand association reaction is constituted so as to include the hybridization buffer tank 10, the probe solution chip 11, the antibody solution tank 12 and the cleaning solution tank 13 and selectively feed a hybridization buffer, a mixed solution of a hybridization buffer and a probe solution, an antibody solution or a cleaning solution into the cartridge 7, 80, 90, it is not absolutely necessary for the apparatus for the receptor-ligand association reaction to include the hybridization buffer tank 10, the probe solution chip 11, the antibody solution tank 12 and the cleaning solution tank 13.

Further, in the above described embodiments, although the apparatus for the receptor-ligand association reaction is constituted so as to recycle a hybridization buffer, a mixed solution of a hybridization buffer and a probe solution, an antibody solution and a cleaning solution through the solution circulation passage 14 into the cartridge 7, 80, 90, the apparatus for the receptor-ligand association reaction may be constituted so that the cleaning solution is discharged through the solution discharge passage 16 without being recycled into the cartridge 7, 80, 90.

Moreover, in the above described embodiments, the apparatus for the receptor-ligand association reaction is constituted so as to clean a number of the absorptive regions 4 of the biochemical analysis unit 1 held in the catridge 7, 80, 90 by repeatedly moving the cleaning solution filling in the inner spaces of the cartridge 7, 80, 90 and the solution circulation pipe 14 through a number of the absorptive regions 4 of the biochemical analysis unit 1 and discharge the cleaning solution through the solution discharge pipe 16, thereby completing the cleaning operation. However, it is possible to repeat the cleaning operation by feeding a new cleaning solution into the cartridge 7, 80, 90 and the solution circulation pipe 14 from the cleaning tank 13 after the cleaning solution was discharged from the cartridge 7, 80, 90 and the solution circulation pipe 14 through the solution discharge pipe 16.

Furthermore, in the embodiment shown in FIGS. 21 to 23, each of the branch passages 83a, 83b, 83c, 83d, . . . , 83n formed in the cartridge 80 includes the folded passages 83aa, 83ab, . . . , 83am; 83ba, 83bb, . . . , 83bm; 83ca, 83cb, . . . , 83cm; . . . , 83na, 83nb, . . . , 83nm for leading a solution to the absorptive regions 4 of the biochemical analysis unit 1 held in the cartridge 80. However, similarly to the embodiment shown in FIGS. 24 to 26, instead of the folded passages 83aa, 83ab, . . . , 83am; 83ba, 83bb, . . . , 83bm; 83ca, 83cb, . . . , 83cm; . . . , 83na, 83nb, . . . , 83nm, through passages may be provided in each of the branch passages 83a, 83b, 83c, 83d, . . . , 83n in such a manner that each of odd numbered through passages from the upstream with respect to the solution flowing direction can feed a solution so as to pass through the corresponding absorptive region 4 downward and that each of even numbered through passages from the upstream with respect to the solution flowing direction can feed a solution so as to pass through the corresponding absorptive region 4 upward.

Moreover, in the embodiment shown in FIGS. 24 to 26, a portion of the solution passage 93 corresponding to each column of the absorptive regions 4 is formed in the cartridge 90 so that each of odd numbered through passages 94 from the upstream with respect to the solution flowing direction can feed a solution so as to pass through a corresponding absorptive region 4 downward and that each of even numbered through passages from the upstream with respect to the solution flowing direction can feed a solution so as to pass through a corresponding absorptive region 4 upward. However, similarly to the embodiment shown in FIGS. 21 to 23, instead of the through passages 94, folded passages may be provided in the portion of the solution passage 93 corresponding to each column of the absorptive regions 4 for leading a solution to the absorptive regions 4 of the biochemical analysis unit 1.

Furthermore, in the above described embodiments, although about 10,000 substantially circular absorptive regions 4 having a size of about 0.01 mm² are formed in the substrate 2 of the biochemical analysis unit 1 in a regular pattern at a density of about 5,000 per cm², the shape of each of the absorptive regions 4 is not limited to a substantially circular shape but may be formed in an arbitrary shape, for example, a rectangular shape.

Moreover, in the above described embodiments, although about 10,000 substantially circular absorptive regions 4 having a size of about 0.01 mm² are formed in the substrate 2 of the biochemical analysis unit 1 in a regular pattern at a density of about 5,000 per cm², the number or size of the absorptive regions 4 may be arbitrarily selected in accordance with the purpose. Preferably, 10 or more of the absorptive regions 4 having a size of 5 mm² or less are formed in the substrate 2 of the biochemical analysis unit 1 at a density of 10/cm² or greater.

Further, in the above described embodiments, although about 10,000 substantially circular absorptive regions 4 having a size of about 0.01 mm² are formed in the biochemical analysis unit 1 in a regular pattern at a density of about 5,000 per cm², it is not absolutely necessary to form the absorptive regions 4 in a regular pattern.

Furthermore, in the above described embodiments, although a number of the absorptive regions 4 of the biochemical analysis unit 1 are formed by charging nylon-6 in a number of the through-hole 3 formed in the substrate 2 made of stainless steel, it is not absolutely necessary to make the substrate 2 of the biochemical analysis unit 1 of stainless steel but the substrate 2 of the biochemical analysis unit 1 may be made of other kinds of material. The substrate 2 of the biochemical analysis unit 1 is preferably made of material capable of attenuating radiation energy and light energy but the material for forming the substrate 2 of the biochemical analysis unit 1 is not particularly limited. The substrate 2 of the biochemical analysis unit 1 can be formed of either inorganic compound material or organic compound material and is preferably formed of a metal material, a ceramic material or a plastic material. Illustrative examples of inorganic compound materials usable for forming the substrate 2 of the biochemical analysis unit 1 and capable of attenuating radiation energy and/or light energy include metals such as gold, silver, copper, zinc, aluminum, titanium, tantalum, chromium, steel, nickel, cobalt, lead, tin, selenium and the like; alloys such as brass, stainless, bronze and the like; silicon materials such as silicon, amorphous silicon, glass, quartz, silicon carbide, silicon nitride and the like; metal oxides such as aluminum oxide, magnesium oxide, zirconium oxide and the like; and inorganic salts such as tungsten carbide, calcium carbide, calcium sulfate, hydroxy apatite, gallium arsenide and the like. High molecular compounds are preferably used as organic compound material for forming the substrate 2 of the biochemical analysis unit 1 and capable of attenuating radiation energy and light energy and illustrative examples thereof include polyolefins such as polyethylene, polypropylene and the like; acrylic resins such as polymethyl methacrylate, polybutylacrylate/polymethyl methacrylate copolymer and the like; polyacrylonitrile; polyvinyl chloride; polyvinylidene chloride; polyvinylidene fluoride; polytetrafluoroethylene; polychlorotrifluoroethylene; polycarbonate; polyesters such as polyethylene naphthalate, polyethylene terephthalate and the like; nylons such as nylon-6, nylon-6,6, nylon-4,10 and the like; polyimide; polysulfone; polyphenylene sulfide; silicon resins such as polydiphenyl siloxane and the like; phenol resins such as novolac and the like; epoxy resin; polyurethane; polystyrene, butadiene-styrene copolymer; polysaccharides such as cellulose, acetyl cellulose, nitrocellulose, starch, calcium alginate, hydroxypropyl methyl cellulose and the like; chitin; chitosan; urushi (Japanese lacquer); polyamides such as gelatin, collagen, keratin and the like; and copolymers of these high molecular materials. These may be a composite compound, and metal oxide particles, glass fiber or the like may be added thereto as occasion demands. Further, an organic compound material may be blended therewith.

Moreover, in the above described embodiments, although a number of the absorptive regions 4 of the biochemical analysis unit 1 are formed by charging nylon-6 in a number of the through-hole 3 formed in the substrate 2 made of stainless steel, it is not absolutely necessary to form a number of the absorptive regions 4 of the biochemical analysis unit 1 of nylon-6 but a number of the absorptive regions 4 of the biochemical analysis unit 1 may be formed of other absorptive material. A porous material or a fiber material may be preferably used as the absorptive material for forming a number of the absorptive regions 4 of the biochemical analysis unit 1 and a number of the absorptive regions 4 of the biochemical analysis unit 1 may be formed by combining a porous material and a fiber material. A porous material for forming a number of the absorptive regions 4 of the biochemical analysis unit 1 may be any type of an organic material or an inorganic material and may be an organic/inorganic composite material. An organic porous material used for forming a number of the absorptive regions 4 of the biochemical analysis unit 1 is not particularly limited but a carbon porous material such as an activated carbon or a porous material capable of forming a membrane filter can be preferably used. Illustrative examples of porous materials capable of forming a membrane filter include nylons such as nylon-6, nylon-6,6, nylon-4,10; cellulose derivatives such as nitrocellulose, acetyl cellulose, butyric-acetyl cellulose; collagen; alginic acids such as alginic acid, calcium alginate, alginic acid/poly-L-lysine polyionic complex; polyolefins such as polyethylene, polypropylene; polyvinyl chloride; polyvinylidene chloride; polyfluoride such as polyvinylidene fluoride, polytetrafluoride; and copolymers or composite materials thereof. An inorganic porous material used for forming a number of the absorptive regions 4 of the biochemical analysis unit 1 is not particularly limited. Illustrative examples of inorganic porous materials preferably usable in the present invention include metals such as platinum, gold, iron, silver, nickel, aluminum and the like; metal oxides such as alumina, silica, titania, zeolite and the like; metal salts such as hydroxy apatite, calcium sulfate and the like; and composite materials thereof. A fiber material used for forming a number of the absorptive regions 4 of the biochemical analysis unit 1 is not particularly limited. Illustrative examples of fiber materials preferably usable in the present invention include nylons such as nylon-6, nylon-6,6, nylon-4,10; and cellulose derivatives such as nitrocellulose, acetyl cellulose, butyric-acetyl cellulose.

Further, in the above described embodiments, although a number of the absorptive regions 4 of the biochemical analysis unit 1 are formed by charging nylon-6 in a number of the through-hole 3 formed in the substrate 2 made of stainless steel, a number of the absorptive regions 4 of the biochemical analysis unit 1 may be formed by pressing an absorptive membrane formed of nylon-6 into a number of the through-holes 3 formed in the substrate 2 made of stainless steel.

Moreover, in the above described embodiments, although a number of the absorptive regions 4 of the biochemical analysis unit 1 are formed by charging nylon-6 in a number of the through-hole 3 formed in the substrate 2 made of stainless steel, a number of the absorptive regions 4 of the biochemical analysis unit 1 may be formed by charging nylon-6 in a number of recesses formed in the substrate of the biochemical analysis unit 1.

Furthermore, in the above described embodiments, although a number of the absorptive regions 4 of the biochemical analysis unit 1 are formed by charging nylon-6 in a number of the through-hole 3 formed in the substrate 2 made of stainless steel, a biochemical analysis unit formed with a number of absorptive regions containing specific binding substances and spaced apart from each other may be formed by spotting a solution containing specific binding substances on regions spaced apart from each other on an absorptive substrate made of an absorptive material.

Furthermore, in the above-described embodiments, a solution containing specific binding substances such as cDNAs are spotted using the spotting device including an injector 5 and a CCD camera 6 so that when the tip end portion of the injector 5 and the center of the absorptive region 4 into which a solution containing specific binding substances is to be spotted are determined to coincide with each other as a result of viewing them using the CCD camera 6, the solution containing the specific binding substances such as cDNA is spotted from the injector 5. However, the solution containing specific binding substances such as cDNAs can be spotted by detecting the positional relationship between a number of the absorptive regions 4 formed in the biochemical analysis unit 1 and the tip end portion of the injector 5 in advance and two-dimensionally moving the biochemical analysis unit 1 or the tip end portion of the injector 5 so that the tip end portion of the injector 5 coincides with each of the absorptive regions 4.

According to the present invention, it is possible to provide a cartridge for a biochemical analysis unit and a method for recording biochemical analysis data in a biochemical analysis unit which can efficiently associate a ligand or a receptor labeled with a labeling substance with receptors or ligands fixed in a plurality of spot-like regions formed in the biochemical analysis unit to be spaced apart from each other, thereby recording biochemical analysis data in the biochemical analysis unit.

1. A cartridge for a biochemical analysis unit being adapted for accommodating a biochemical analysis unit and formed with at least one fluid passage for leading a solution to only a plurality of absorptive regions formed in the biochemical analysis unit to be spaced apart from each other.

2. A cartridge for a biochemical analysis unit in accordance with claim 1 wherein the at least one fluid passage is formed so as to cut through the plurality of absorptive regions formed in the biochemical analysis unit to be spaced apart from each other.

3. A cartridge for a biochemical analysis unit in accordance with claim 1 wherein a plurality of fluid passages are formed.

4. A cartridge for a biochemical analysis unit in accordance with claim 1 wherein the plurality of absorptive regions are two-dimensionally formed in the biochemical analysis unit to be spaced apart from each other and a fluid passage is formed for each line of the plurality of absorptive regions so that the fluid passage can successively lead a solution to the plurality of absorptive regions constituting each line.

5. A cartridge for a biochemical analysis unit in accordance with claim 4 wherein the fluid passage formed for each line of the plurality of absorptive regions is formed so as to sequentially cross the absorptive regions constituting the line.

6. A cartridge for a biochemical analysis unit in accordance with claim 4 wherein the plurality of fluid passages are disposed on one side of the biochemical analysis unit held in the cartridge.

7. A cartridge for a biochemical analysis unit in accordance with claim 1 wherein the plurality of absorptive regions are two-dimensionally formed in the biochemical analysis unit to be spaced apart from each other and a fluid passage is formed for each of the absorptive regions so as to feed a solution thereto.

8. A cartridge for a biochemical analysis unit in accordance with claim 7 wherein the fluid passage formed for each of the absorptive regions is formed so as to cut through the corresponding absorptive region.

9. A cartridge for a biochemical analysis unit in accordance with claim 7 wherein the fluid passage formed for each of the absorptive regions is disposed on one side of the biochemical analysis unit held in the cartridge.

10. A cartridge for a biochemical analysis unit in accordance with claim 2 wherein a portion of the fluid passage facing the absorptive region of the biochemical analysis unit has a cross sectional area of 0.2 mm² or less.

11. A cartridge for a biochemical analysis unit in accordance with claim 5 wherein a portion of the fluid passage facing the absorptive region of the biochemical analysis unit has a cross sectional area of 0.2 mm² or less.

12. A cartridge for a biochemical analysis unit in accordance with claim 8 wherein a portion of the fluid passage facing the absorptive region of the biochemical analysis unit has a cross sectional area of 0.2 mm² or less.

13. A cartridge for a biochemical analysis unit in accordance with claim 2 wherein a portion of the fluid passage facing the absorptive region of the biochemical analysis unit has a cross sectional area of 0.5 mm² or less.

14. A cartridge for a biochemical analysis unit in accordance with claim 5 wherein a portion of the fluid passage facing the absorptive region of the biochemical analysis unit has a cross sectional area of 0.5 mm² or less.

15. A cartridge for a biochemical analysis unit in accordance with claim 8 wherein a portion of the fluid passage facing the absorptive region of the biochemical analysis unit has a cross sectional area of 0.5 mm² or less.

16. A method for recording biochemical analysis data in a biochemical analysis unit comprising the steps of accommodating a biochemical analysis unit including a substrate formed with a plurality of absorptive regions to be spaced apart from each other in which receptors or ligands are fixed in a cartridge and feeding a reaction solution containing a ligand or a receptor labeled with a labeling substance only to the absorptive regions of the biochemical analysis unit through at least one fluid passage formed in the cartridge, thereby selectively associating the ligand or the receptor contained in the reaction solution with the receptors or the ligands fixed in the plurality of the absorptive regions of the biochemical analysis unit.

17. A method for recording biochemical analysis data in a biochemical analysis unit in accordance with claim 16 which further comprises the step of feeding a cleaning solution only to the absorptive regions of the biochemical analysis unit through at least one fluid passage formed in the cartridge, thereby cleaning the plurality of absorptive regions of the biochemical analysis unit in which the receptors or the ligands are fixed with the cleaning solution.

18. A method for recording biochemical analysis data in a biochemical analysis unit in accordance with claim 16 wherein the at least one fluid passage is formed in the cartridge so as to cut through the plurality of absorptive regions formed in the biochemical analysis unit to be spaced apart from each other.

19. A method for recording biochemical analysis data in a biochemical analysis unit in accordance with claim 17 wherein the at least one fluid passage is formed in the cartridge so as to cut through the plurality of absorptive regions formed in the biochemical analysis unit to be spaced apart from each other.

20. A method for recording biochemical analysis data in a biochemical analysis unit in accordance with claim 18 wherein a plurality of fluid passages are formed in the cartridge correspondingly to the plurality of absorptive regions formed in the biochemical analysis unit.

21. A method for recording biochemical analysis data in a biochemical analysis unit in accordance with claim 19 wherein a plurality of fluid passages are formed in the cartridge correspondingly to the plurality of absorptive regions formed in the biochemical analysis unit.

22. A method for recording biochemical analysis data in a biochemical analysis unit in accordance with claim 16 wherein the plurality of absorptive regions are two-dimensionally formed in the biochemical analysis unit to be spaced apart from each other and a fluid passage is formed in the cartridge for each line of the plurality of absorptive regions so that the fluid passage can successively lead a solution to the plurality of absorptive regions constituting each line.

23. A method for recording biochemical analysis data in a biochemical analysis unit in accordance with claim 17 wherein the plurality of absorptive regions are two-dimensionally formed in the biochemical analysis unit to be spaced apart from each other and a fluid passage is formed in the cartridge for each line of the plurality of absorptive regions so that the fluid passage can successively lead a solution to the plurality of absorptive regions constituting each line.

24. A method for recording biochemical analysis data in a biochemical analysis unit in accordance with claim 22 wherein the fluid passage formed for each line of the plurality of absorptive regions is formed so as to sequentially cut through the absorptive regions constituting the line.

25. A method for recording biochemical analysis data in a biochemical analysis unit in accordance with claim 23 wherein the fluid passage formed for each line of the plurality of absorptive regions is formed so as to sequentially cut through the absorptive regions constituting the line.

26. A method for recording biochemical analysis data in a biochemical analysis unit in accordance with claim 22 wherein the plurality of fluid passages are formed in the cartridge so as to be disposed on one side of the biochemical analysis unit held in the cartridge.

27. A method for recording biochemical analysis data in a biochemical analysis unit in accordance with claim 23 wherein the plurality of fluid passages are formed in the cartridge so as to be disposed on one side of the biochemical analysis unit held in the cartridge.

28. A method for recording biochemical analysis data in a biochemical analysis unit in accordance with claim 16 wherein the plurality of absorptive regions are two-dimensionally formed in the biochemical analysis unit to be spaced apart from each other and a fluid passage is formed in the cartridge for each of the absorptive regions so as to feed a solution thereto.

29. A method for recording biochemical analysis data in a biochemical analysis unit in accordance with claim 17 wherein the plurality of absorptive regions are two-dimensionally formed in the biochemical analysis unit to be spaced apart from each other and a fluid passage is formed in the cartridge for each of the absorptive regions so as to feed a solution thereto.

30. A method for recording biochemical analysis data in a biochemical analysis unit in accordance with claim 28 wherein the fluid passage formed for each of the absorptive regions is formed so as to cut through the corresponding absorptive region.

31. A method for recording biochemical analysis data in a biochemical analysis unit in accordance with claim 29 wherein the fluid passage formed for each of the absorptive regions is formed so as to cut through the corresponding absorptive region.

32. A method for recording biochemical analysis data in a biochemical analysis unit in accordance with claim 28 wherein the fluid passage formed for each of the absorptive regions is disposed on one side of the biochemical analysis unit held in the cartridge.

33. A method for recording biochemical analysis data in a biochemical analysis unit in accordance with claim 29 wherein the fluid passage formed for each of the absorptive regions is disposed on one side of the biochemical analysis unit held in the cartridge.

34. A method for recording biochemical analysis data in a biochemical analysis unit in accordance with claim 18 wherein the at least one fluid passage is formed in the cartridge so that a portion thereof facing the absorptive region of the biochemical analysis unit has a cross sectional area of 0.2 mm² or less.

35. A method for recording biochemical analysis data in a biochemical analysis unit in accordance with claim 19 wherein the at least one fluid passage is formed in the cartridge so that a portion thereof facing the absorptive region of the biochemical analysis unit has a cross sectional area of 0.2 mm² or less.

36. A method for recording biochemical analysis data in a biochemical analysis unit in accordance with claim 24 wherein the fluid passage formed for each line of the plurality of absorptive regions is formed so that a portion thereof facing the absorptive region of the biochemical analysis unit has a cross sectional area of 0.2 mm² or less.

37. A method for recording biochemical analysis data in a biochemical analysis unit in accordance with claim 25 wherein the fluid passage formed for each line of the plurality of absorptive regions is formed so that a portion thereof facing the absorptive region of the biochemical analysis unit has a cross sectional area of 0.2 mm² or less.

38. A method for recording biochemical analysis data in a biochemical analysis unit in accordance with claim 30 wherein the fluid passage formed for each of the absorptive regions is formed so that a portion thereof facing the absorptive region of the biochemical analysis unit has a cross sectional area of 0.2 mm² or less.

39. A method for recording biochemical analysis data in a biochemical analysis unit in accordance with claim 31 wherein the fluid passage formed for each of the absorptive regions is formed so that a portion thereof facing the absorptive region of the biochemical analysis unit has a cross sectional area of 0.2 mm² or less.

40. A method for recording biochemical analysis data in a biochemical analysis unit in accordance with claim 26 wherein the at least one fluid passage is formed in the cartridge on one side of the biochemical analysis unit held in the cartridge so that a portion thereof facing the absorptive region of the biochemical analysis unit has a length of 0.5 mm or less.

41. A method for recording biochemical analysis data in a biochemical analysis unit in accordance with claim 27 wherein the at least one fluid passage is formed in the cartridge on one side of the biochemical analysis unit held in the cartridge so that a portion thereof facing the absorptive region of the biochemical analysis unit has a length of 0.5 mm or less.

42. A method for recording biochemical analysis data in a biochemical analysis unit in accordance with claim 32 wherein the fluid passage formed for each of the absorptive regions is disposed on one side of the biochemical analysis unit held in the cartridge so that a portion thereof facing the absorptive region of the biochemical analysis unit has a length of 0.5 mm or less.

43. A method for recording biochemical analysis data in a biochemical analysis unit in accordance with claim 33 wherein the fluid passage formed for each of the absorptive regions is disposed on one side of the biochemical analysis unit held in the cartridge so that a portion thereof facing the absorptive region of the biochemical analysis unit has a length of 0.5 mm or less.

44. A method for recording biochemical analysis data in a biochemical analysis unit in accordance with claim 16 wherein the substrate of the biochemical analysis unit is formed with a plurality of holes to be spaced apart from each other and the plurality of absorptive regions of the biochemical analysis unit are formed by charging an absorptive material in the plurality of holes formed in the substrate.

45. A method for recording biochemical analysis data in a biochemical analysis unit in accordance with claim 16 wherein the substrate of the biochemical analysis unit is formed with 10 or more absorptive regions.

46. A method for recording biochemical analysis data in a biochemical analysis unit in accordance with claim 16 wherein each of the plurality of absorptive regions formed in the substrate of the biochemical analysis unit has a size of less than 5 mm².

47. A method for recording biochemical analysis data in a biochemical analysis unit in accordance with claim 16 wherein the plurality of absorptive regions are formed in the substrate of the biochemical analysis unit at a density of 10 or more per cm².

48. A method for recording biochemical analysis data in a biochemical analysis unit in accordance with claim 16 wherein the substrate of the biochemical analysis unit has a property of attenuating radiation energy.

49. A method for recording biochemical analysis data in a biochemical analysis unit in accordance with claim 48 wherein the substrate of the biochemical analysis unit has a property of reducing the energy of radiation to ⅕ or less when the radiation travels in the substrate by a distance equal to that between neighboring absorptive regions.

50. A method for recording biochemical analysis data in a biochemical analysis unit in accordance with claim 16 wherein the substrate of the biochemical analysis unit has a property of attenuating light energy.

51. A method for recording biochemical analysis data in a biochemical analysis unit in accordance with claim 50 wherein the substrate of the biochemical analysis unit has a property of reducing the energy of light to ⅕ or less when the light travels in the substrate by a distance equal to that between neighboring absorptive regions.
